SEQUENCE LISTING (24 sequences)

SEQ ID NO: 1 - DNA, 27 bp, synthetic construct
  misc_feature 1..27: note = Description of sequence: oligonucleotide
  source 1..27: mol_type = other DNA
ttttccataggctccgcccccctgacg

SEQ ID NO: 2 - DNA, 26 bp, synthetic construct
  misc_feature 1..26: note = Description of sequence: oligonucleotide
  source 1..26: mol_type = other DNA
cttttcggggaaatgtgcgcggaacc

SEQ ID NO: 3 - DNA, 56 bp, synthetic construct
  misc_feature 1..56: note = Description of sequence: oligonucleotide
  source 1..56: mol_type = other DNA
ggttccgcgcacatttccccgaaaagcagcctagccaagtaaatagaccatcatgc

SEQ ID NO: 4 - DNA, 60 bp, synthetic construct
  misc_feature 1..60: note = Description of sequence: oligonucleotide
  source 1..60: mol_type = other DNA
gtggcggcacgcgtaagtataagacaaaagaagtggcagttgtagacatataagaaatat

SEQ ID NO: 5 - DNA, 58 bp, synthetic construct
  misc_feature 1..58: note = Description of sequence: oligonucleotide
  source 1..58: mol_type = other DNA
aagatgggcgggagtcttctgggcaggccatggcactaggactaaaggttggccaaag

SEQ ID NO: 6 - DNA, 55 bp, synthetic construct
  misc_feature 1..55: note = Description of sequence: oligonucleotide
  source 1..55: mol_type = other DNA
tcaggggggcggagcctatggaaaagtactccaatcagaagaaacagccagtgcc

SEQ ID NO: 7 - DNA, 124 bp, synthetic construct
  misc_feature 1..124: note = Description of sequence: oligonucleotide
  source 1..124: mol_type = other DNA
cttttgtcttatacttacgcgtgccgccacctcgagaattcgagctcggtacccggggatcctctagagtcgacctgcaggcatgcaagcttatcatgggcccatctcgacctacttcgtatag

SEQ ID NO: 8 - DNA, 124 bp, synthetic construct
  misc_feature 1..124: note = Description of sequence: oligonucleotide
  source 1..124: mol_type = other DNA
ctatacgaagtaggtcgagatgggcccatgataagcttgcatgcctgcaggtcgactctagaggatccccgggtaccgagctcgaattctcgaggtggcggcacgcgtaagtataagacaaaag

SEQ ID NO: 9 - DNA, 57 bp, synthetic construct
  misc_feature 1..57: note = Description of sequence: oligonucleotide
  source 1..57: mol_type = other DNA
ctcgagaattcgagctcggtacccggggttagagtaattcatacaaaaggactcgcc

SEQ ID NO: 10 - DNA, 21 bp, synthetic construct
  misc_feature 1..21: note = Description of sequence: oligonucleotide
  source 1..21: mol_type = other DNA
gctgctcgacacgagctcacg

SEQ ID NO: 11 - DNA, 49 bp, synthetic construct
  misc_feature 1..49: note = Description of sequence: oligonucleotide
  source 1..49: mol_type = other DNA
gtgtcgtgagctcgtgtcgagcagcgccgccaccatggagaccgacacc

SEQ ID NO: 12 - DNA, 24 bp, synthetic construct
  misc_feature 1..24: note = Description of sequence: oligonucleotide
  source 1..24: mol_type = other DNA
ggggccggggttctcctccacgtc

SEQ ID NO: 13 - DNA, 49 bp, synthetic construct
  misc_feature 1..49: note = Description of sequence: oligonucleotide
  source 1..49: mol_type = other DNA
gacgtggaggagaaccccggccccatggagcttcttagtgggcctcacg

SEQ ID NO: 14 - DNA, 55 bp, synthetic construct
  misc_feature 1..55: note = Description of sequence: oligonucleotide
  source 1..55: mol_type = other DNA
agcttgcatgcctgcaggtcgactctagaggcgttggccgattcattaatgcagg

SEQ ID NO: 15 - DNA, 25 bp, synthetic construct
  misc_feature 1..25: note = Description of sequence: oligonucleotide
  source 1..25: mol_type = other DNA
caccgttagtcctagtgccatgaag

SEQ ID NO: 16 - DNA, 25 bp, synthetic construct
  misc_feature 1..25: note = Description of sequence: oligonucleotide
  source 1..25: mol_type = other DNA
aaaccttcatggcactaggactaac

SEQ ID NO: 17 - DNA, 60 bp, synthetic construct
  misc_feature 1..60: note = Description of sequence: oligonucleotide
  source 1..60: mol_type = other DNA
tagagatcgtcgagaattcgagctcggtacgccgccaccatgagcgagctgattaaggag

SEQ ID NO: 18 - DNA, 56 bp, synthetic construct
  misc_feature 1..56: note = Description of sequence: oligonucleotide
  source 1..56: mol_type = other DNA
acttcctctgccctctcctccggacccgccgccattaagcttgtgccccagtttgc

SEQ ID NO: 19 - DNA, 60 bp, synthetic construct
  misc_feature 1..60: note = Description of sequence: oligonucleotide
  source 1..60: mol_type = other DNA
tgggccaggattctcctccacgtcaccgcatgttagaagacttcctctgccctctcctcc

SEQ ID NO: 20 - DNA, 51 bp, synthetic construct
  misc_feature 1..51: note = Description of sequence: oligonucleotide
  source 1..51: mol_type = other DNA
tgcggtgacgtggaggagaatcctggcccaatggctcctctcggatacctc

SEQ ID NO: 21 - DNA, 51 bp, synthetic construct
  misc_feature 1..51: note = Description of sequence: oligonucleotide
  source 1..51: mol_type = other DNA
gcaggtcgactctagaggatccccgggtacctacttgcaggtgtgcacgtc

SEQ ID NO: 22 - DNA, 24 bp, synthetic construct
  misc_feature 1..24: note = Description of sequence: oligonucleotide
  source 1..24: mol_type = other DNA
caccgtcgagtcgcttctcgatta

SEQ ID NO: 23 - DNA, 24 bp, synthetic construct
  misc_feature 1..24: note = Description of sequence: oligonucleotide
  source 1..24: mol_type = other DNA
aaactaatcgagaagcgactcgac

SEQ ID NO: 24 - DNA, 20 bp, synthetic construct
  misc_feature 1..20: note = Description of sequence: oligonucleotide
  source 1..20: mol_type = other DNA
gactatcatatgcttaccgt

(19) United States
(12) Patent Application Publication
(10) Pub. No.: US 2026/0297532 A1
(43) Pub. Date: Oct. 1, 2026

(54) COMPOSITIONS AND METHODS FOR CONTROLLING PATTERNING OF HUMAN KIDNEY ORGANOID NEPHRONS

(71) Applicant: UNIVERSITY OF SOUTHERN CALIFORNIA, Los Angeles, CA (US)

(72) Inventors: Connor-James Fausto, Los Angeles, CA (US); Fokion Glykofrydis, Los Angeles, CA (US); Nils Lindstrom, Los Angeles, CA (US); Leonardo Morsut, Los Angeles, CA (US)

(21) Appl. No.: 19/396,812

(22) Filed: Nov. 21, 2025

Related U.S. Application Data
(60) Provisional application No. 63/725,054, filed on Nov. 26, 2024.

(51) Int. Cl. C12N 5/071; C07K 14/47; C12N 5/074

(52) U.S. Cl. CPC C12N 5/0686; C07K 14/4705; C07K 14/4728; C12N 5/0607; C12N 2513/00

(57) ABSTRACT

The present disclosure relates to compositions, systems, and methods for controlling patterning of human kidney organoid nephrons.

### CROSS-REFERENCE TO RELATED APPLICATIONS

This application claims the benefit of priority to U.S. Provisional Application No. 63/725,054, filed Nov. 26, 2024, which is incorporated by reference herein in its entirety.

### STATEMENT REGARDING FEDERALLY SPONSORED RESEARCH

This invention was made with government support under Grant Nos. DK136802 and GM138256 awarded by the National Institutes of Health, and under Grant Nos. 2034495 and 2145528, awarded by the National Science Foundation. The government has certain rights in the invention.

### REFERENCE TO SEQUENCE LISTING

The sequence listing submitted on Nov. 21, 2025, as an .XML file entitled “11760-021US1_ST26.xml” created on Nov. 19, 2025, and having a file size of 31,768 bytes is hereby incorporated by reference pursuant to 37 C.F.R. § 1.52(e)(5).

### FIELD

The present disclosure relates to compositions, systems, and methods for controlling patterning of human kidney organoid nephrons.

### BACKGROUND

End-stage renal disease (ESRD) is a prevalent and unsolved medical problem. The only treatment options for ESRD are dialysis or transplantation. Dialysis diminishes quality of life and involves significant health risks (for example, depression, infection, heart failure), but is often the only viable option due to an imbalance in the demand and supply of donor kidneys available for transplantation.

What is needed are new methods for patterning of human kidney organoid nephrons for kidney tissue replacement therapies to address the shortage of transplantable kidneys.

### SUMMARY

Disclosed herein are compositions, systems, and methods for controlling the patterning of human kidney organoid nephrons.

Accordingly, in some aspects, disclosed herein is a synthetic organizer comprising one or more cells, wherein the one or more cells comprise a nucleic acid construct comprising a WNT3A gene or a WNT9B gene under the control of an inducible promoter.

In some aspects, also disclosed herein is a system comprising a synthetic organizer and a human kidney organoid nephron, wherein the synthetic organizer facilitates patterning of the human kidney organoid nephron.

In some aspects, also disclosed herein is a method of patterning a human kidney organoid nephron, comprising: obtaining a human kidney organoid nephron; and contacting the human kidney organoid nephron with a synthetic organizer.

### BRIEF DESCRIPTION OF THE DRAWINGS

The accompanying figures, which are incorporated in and constitute a part of this specification, illustrate several aspects described below.

FIGS. 1A-1M show human nephrons develop multi-axial patterning and morphogenesis relative to collecting duct-localized WNT ligands. FIG. 1A shows seqFISH spatial transcriptomic analyses of week 16 developing human kidney and nephrogenesis (MM: metanephric mesenchyme, CD: collecting duct). Scale bar is 500 μm. FIG. 1B shows a schematic of nephrogenesis and CD expressed WNT ligands (WNT11: CD tip; WNT9B: CD stalk) and nephron expressed WNT4, proposed β-catenin signaling, and cell populations based on coloring and labels. FIG. 1C shows transcript distribution at the pretubular aggregate (PTA) stage (middle panels), schematics showing anatomies (left), inferred cell-populations and CD adjacent-distant (AD) and proximal-distal (PD) axes (right). FIG. 1D shows graphs showing distance between the CD surface and transcripts in nephron progenitors (NPCs) at week 13 and 16 (left; CD adjacent: CD-A; CD-distant: CD-D) and transcript distribution across CD-A to CD-D axis in the PTA (right). cyan dot: WNT11-to-WNT9B transition; yellow dot: center of RV; red lines: standard deviation. FIG. 1E shows transcript distribution at the renal vesicle (RV) stage with schematics as in FIG. 1C. FIG. 1F shows WNT4 transcript polarization in RVs relating to WNT11 and WNT9B with radar plots depicting transcript frequency distribution (cyan dot: WNT11-to-WNT9B transition; yellow dot: center of RV; red lines: standard deviation). FIGS. 1G-1H show transcript distribution at the comma-shaped (CSB) and S-shaped body (SSB) stages with schematics outlining cell populations, and PD and AD axes. Scale bars in C to H are 25 μm. FIG. 11 shows a UMAP plot of snRNAseq data containing CD tip and stalk, and nephrogenic NPC to RV-SSB cell populations. FIG. 1J shows a UMAP plot showing probability of canonical WNT sender/receiver pairings. FIGS. 1K-1L show snRNAseq populations and signal-interactions mapped to spatial coordinates (FIG. 1L contains boxed regions from FIG. 1K). FIG. 1M shows expression of ligand (WNT9B) and responding cells (WNT4 and FGF8 positive) near the CD-tip (WNT11), in boxed regions with receivers (red and pink) in positions of NPCs and PTA/RVs. Scale bars in FIGS. 1K-1M are 500 μm.

FIGS. 2A-2E show spatial transcriptomic data of human developing nephrons (in support to FIGS. 1A-1H). FIGS. 2A-2D show Xenium spatial transcriptomic data examples of developing human nephrons at the pretubular aggregate (FIG. 2A), renal vesicle (FIG. 2B), comma-shaped body (FIG. 2C), and S-shaped body (FIG. 2D) nephron stages with genes and colors shown as in FIGS. 1A-1H. FIG. 2E shows seqFISH spatial transcriptomic single channels of whole human kidney sections with genes shown as in FIG. 1A. Genes and colors as shown on panel. Scale bars in FIGS. 2A-2D are 50 μm while those in FIG. 2E are 500 μm.

FIGS. 3A-3E show spatial transcriptomic data breakdown and analysis (in support to FIGS. 1A-1H). FIG. 3A shows single-channel breakdown of overlayed spatial transcriptomic data at the pretubular aggregate stage shown in FIG. 1C. FIG. 3B shows a method for quantifying distances and distribution between various gene transcripts (dots) and the CD surface (dotted white line) in nephron progenitors and pretubular staged nephrons from 13.5 and 16.5 week human developing kidney sections, related to FIG. 1D. FIGS. 3C-3E show single channels for renal vesicle (FIG. 3C), comma-shaped (FIG. 3D), and S-shaped body (FIG. 3E) stages of nephrogenesis. Scale bars are 25 μm.

FIGS. 4A-4I show serial subsetting of Week 15-17 human kidney snRNA-seq data and subsequent WNT pathway ligand-receptor pairing analysis (in support to FIGS. 1I-1M). FIG. 4A shows snRNA-seq dataset of weeks 15-17 human kidneys. FIG. 4B shows quality control metrics of the week 15-17 snRNA-seq dataset. FIG. 4C shows an overlay plot of origin UMAP grouped by kidney cell types with dotted lines indicating nuclei subset for a focused analysis. FIG. 4D shows a subset UMAP after renormalization and reclustering; with insert of the cell type grouping same as in FIG. 11. FIG. 4E shows FeaturePlot expression of nephron progenitor (ELAVL4, MEOX1, PCDH15), early nephron (FGF8, LHX1, WNT4, JAG1, EMX2, KRT8), distal precursor (POU3F3, TFAP2A), pre-connecting tubule (GATA3), early epithelial nephron cell (HNF1B, DLL1), proximal precursor (HNF4A), and podocyte precursor (MAFB, WT1, ZO1 enriched in UMAP clusters 9, 14, 18). FIG. 4F shows collecting duct stalk (AQP2, WNT9B), and collecting duct tip (RET, WNT11) cell types. FIGS. 4G-4H show DotPlot expression of nephron patterning markers (FIG. 4G) and WNT ligands (FIG. 4H) implicated in kidney development, Frizzle receptors 1-10, LRP receptors 5/6, and R-spondin 1-4 potentiators of WNT signaling. FIG. 4I shows WNT pathway ligand-receptor pairing analyses via CellChat of the early nephron and CD snRNA-seq data, with blue and red arrows indicating the top WNT sender (clusters 5 and 1) and WNT receiver (clusters 19 and 17) clusters.

FIGS. 5A-5F show integration between week 15-17 whole human kidney snRNAseq dataset to two seqFISH datasets and label transfer from snRNA-seq to seqFISH data (in support to FIGS. 1I-1M). FIG. 5A shows a single nuclear (sn) RNA-seq UMAP of weeks 15-17 human kidneys and spatial maps of weeks 16.5 and 13.5 human kidneys. FIG. 5B shows an integrated UMAP of snRNA-seq and seqFISH datasets in overlay and split views. FIG. 5C shows an integrated UMAP of snRNA-seq dataset grouped by kidney cell types with dotted lines indicating nuclei subset for the focused analysis labeled by re-clustering after subsetting (FIGS. 4C-4D). FIG. 5D shows an integrated UMAP and spatial maps of the week 16.5 human kidney seqFISH dataset with snRNA-seq labels (Seurat clusters) transferred or filtered out (unclear) using nearest neighbor analysis with k=30 nearest neighbors. FIGS. 5E-5F show spatial maps of snRNA-seq labels (celltypes) transferred as an overlay and split cell types (FIG. 5E), with dotted lines indicating zoomed regions to display transferred labels along the cortical-medullary axis (FIG. 5F).

FIGS. 6A-6I show human developing kidney single-cell RNA sequencing analyses (in support to FIGS. 1I-1M). FIG. 6A shows a single-cell RNA sequencing UMAP showing clustering of nephrogenic populations and collecting duct. FIG. 6B shows expression of key nephron markers in the UMAP shown in FIG. 6A, showing self-renewing progenitors (CITED1), epithelializing tubular precursors in (KRT8), developing renal vesicle and medial precursors (JAG1), differentiating progenitors (WNT4), podocytes and nephron progenitors (WT1), and epithelial tubule populations (CDH1). FIGS. 6C-6D show focused analysis of subset shown indicated by dotted line in FIG. 6A after renormalization and reclustering (FIG. 6C) and annotated by cell type based on gene expression of nephron progenitors (ELAVL4, MEOX1, PCDH15), early nephron (FGF8, LHX1, WNT4, JAG1, EMX2, KRT8), distal precursors (POU3F3, TFAP2A; indicated by dotted cluster n12), connecting tubule precursors (GATA3; indicated by dotted cluster n12), collecting duct stalk (WNT9B, AQP2; indicated by dotted clusters cd0, cd5, cd1) (FIG. 6D). FIG. 6E shows early epithelializing nephron (HNF1B, DLL1), proximal precursors (HNF4A) and podocyte precursors (MAFB, WT1, ZO1; indicated by dotted cluster n11). FIGS. 6F-6G show DotPlot expression of nephron patterning markers (FIG. 6F) and WNT ligands implicated in kidney development, Frizzle receptors 1-10, LRP receptors 5/6, and R-spondin 1-4 potentiators of WNT signaling (FIG. 6G). FIGS. 6H-6I show WNT pathway ligand-receptor pairing analyses via CellChat of the early nephron and CD scRNA-seq data.

FIGS. 7A-7M show kidney organoid nephrons form without intrinsic asymmetry but can respond to cell-secreted localized WNT ligands. FIG. 7A shows an overview of kidney organoid differentiation protocol, depicting formation of nephron-like structures in the absence of CD, noting apical-basal polarity, initial lack of PD-polarity, no AD-polarity. FIG. 7B shows organoid immunostains showing condensing PTA-like structures (Day10), lumenized epithelial RV-like structures (Day12); stains and scale bars as indicated. FIG. 7C shows bulk transcript counts comparisons between kidney organoids, developing human kidneys, and iPSCs. FIGS. 7D-7E show UMAP plots showing organoid scRNAseq for days 10, 12, and 14, with lineage and differentiation markers shown as feature plots. FIG. 7F shows schema showing strategy for providing localized canonical WNT ligands to kidney organoids via genetically engineered HEK cell organizers. FIG. 7G shows Day14 organoids coupled with control or WNT secreting organizers immunostained as shown. Right panels show magnification of left panel inlets (white square); dotted lines demarcating organizers. Scale bar is 500 μm. FIGS. 7H-7I show graphs showing nephron areas and quantified immunofluorescence. FIGS. 7J-7L show Day 14 organoids coupled with WNT-secreting organizers and simultaneously exposed to IWR1 or IWP2, schema of experiments, graphs displaying HNF4AYFP positive nephrons near organizer. Scale bar is 500 μm. FIG. 7M shows bulk transcript count comparisons of Day14 organizer-coupled organoid regions for controls, WNT-secreting organizers, and non-coupled organoids.

FIGS. 8A-8I show limited Wnt/β-catenin interactions in human kidney organoids and WNT alternative peptide treatment. FIG. 8A shows UMAP plots of day 10-14 human kidney organoids identify nephrogenic cells transitioning from progenitor-like to pretubular aggregate-like to renal vesicle-like profiles in the presence of interstitium, with dotted lines indicating nephron cell types. FIG. 8B shows gene expression of key markers enriched in self-renewing progenitors (CITED1), early differentiating progenitors (PCDH15, MEOX1, ELAVL4), the committed renal vesicle (JAG1, LHX1) and distal precursors (EMX2, POU3F3, GATA3) in the UMAP shown in FIG. 8A. FIG. 8C shows expression of WNT ligands implicated in kidney development, Frizzled1-10 receptors, LRP5/6 co-receptors, and RSPO1-4 potentiators of Wnt/β-catenin activity across nephrogenic and interstitial clusters of kidney organoids. FIG. 8D shows a nephron subset after reclustering and gene expression of self-renewing progenitors (CITED1), podocyte precursors (MAFB), early epithelial nephron (HNF1B), proximal precursors (HNF4A), and WNT targets (DKK1, AXIN2). FIG. 8E shows histograms comparing number of cells with expression of AXIN2, WNT4, and FGF8 between developing human kidney and kidney organoid single-cell transcriptomic datasets. FIGS. 8F-8H show images of organoids with epithelializing nephron structures after nephrogenesis induction via CHIR, PG008, or DMSO which shows day 10 kidney organoids with epithelializing pretubular aggregate-like nephrons (FIG. 8F), day 14 HNF4A-YFP expression (FIG. 8G), days 10 and 14 immunostains of nephron patterning markers (WT1, JAG1, CDH1) (FIG. 8H). FIG. 8I shows images of organoids with epithelializing nephron structures after nephrogenesis induction via CHIR, followed by treatment for 2 days with the indicated WNT inhibitors, fixed and stained at day 10 for the markers WT1, JAG1, CDH1 and DAPI. Scale bar is 500 μm in FIGS. 8F-8I.

FIGS. 9A-9E show WNT gradient reconstitution via synthetic organizers on kidney explants and kidney organoids. FIG. 9A, left, shows a schematic of genetic constructs in the HEK organizer cells. FIG. 9A, right, shows an outline of supplemental experiment testing the ability of synthetic organizers to influence nephron development programs, determined by inducing ectopic nephrogenesis in mouse kidney explants. FIG. 9B shows immunostain examples of conjugated organizers and mouse kidney explants, fixed and stained for WT1, JAG1 and CDH1/3, scale bar is 500 μm. Right, quantifications of ectopic nephrons expressing JAG1; a Jag-positive nephron is score as ectopic if it is found in the mouse metanephric mesenchyme adjacent to WNT organizers, but not in contact with the collecting duct. FIG. 9C shows immunostained and tissue cleared day 14 organoids coupled with constitutive Wnt3a organizers or controls. Boxed regions are those shown at higher magnification. Dotted free hand lines mark the organizer. Scale bar is 500 μm. FIGS. 9D-9E show Day 18 organoids coupled with constitutive Wnt3a organizers or controls immunostained with antibody against LEF1, scale bar is 500 μm (FIG. 9D) and quantification over distance from organizer of LEF1 intensity measured radially from edge of organizer (FIG. 9E), revealing a long-range β-catenin activity gradient with halfway to max of 263.3 um.

FIGS. 10A-10M show nephron polarization driven by bioengineered dox-tunable WNT secreting synthetic organizers. FIGS. 10A-10B show dose-response curves showing geometric mean fluorescence intensity of the WNT-reporting TagBFP module (top panel) and the cadherin-reporting mCherry^{Surface} module (bottom panel) against 10-fold incremental increases in doxycycline concentration. Dose-response curves show results for iWNT9B organizer (FIG. 10A) and iWnt3a organizer (FIG. 10B) each harboring different cadherin modules as indicated. FIG. 10C shows TagBFP fluorescence histograms of iWnt3a organizer cells after 24 h exposure to indicated doxycycline concentrations. FIG. 10D shows synthetic organizer genetic constructs: mCherry^{Surface}-reported, constitutive Cdh3 expression cassette is integrated in the ROGI1 locus; TagBFP-reported, doxycycline gated WNT expression is integrated in the ROSA26 locus. FIGS. 10E-10F show live fluorescence imaging of co-cultures between iWnt3a (FIG. 10E) or iWNT9B (FIG. 10F) cells with β-catenin reporter eGFP mouse embryonic stem cells. Green signal is from the activated reporter cells; blue signal is from the induced WNT cassette. Scale bar is 100 μm. FIG. 10G shows fluorescent microscope images of mouse kidney explants conjugated with iWNT9B synthetic organizers at the indicated doxycycline doses; left, shows fluorescence from live imaging from constitutive mCherry (red) and inducible TagBFP (blue); right, samples fixed and stained for WT1, JAG1 and CDH1, where ectopic JAG1 positive nephrons are indicated with a white arrowhead, and quantified on the far right. Scale bar is 500 μm. FIGS. 10H-10I show percentage of nephrons showing polarized EMX2/POU3F3 phenotype in Day 14 organoids (FIG. 10H) and their length (FIG. 10I), for renal vesicles around and 2-3 layers away from iWnt3a-SOs, at the indicated doses of doxycycline. Corresponding sample images are shown in FIG. 11C. FIGS. 10J-10K show quantification of POU3F3 (FIG. 10J) and EMX2 (FIG. 10K) intensities along the organizer adjacent-distant axis in iWnt3a-SO polarized nephrons at the indicated doxycycline doses. Corresponding sample image is shown in FIG. 11E. FIGS. 10L-10N show bar graphs of the area of the indicated markers DLL1, JAG1 and WT1, as shown in FIGS. 11J-11L, here inclusive of p-values.

FIGS. 11A-11L show organoid nephrons distalize and align their nascent axial polarities towards tunable WNT-secreting organizers. FIG. 11A shows an overview of doxycycline-inducible WNT synthetic organizers that report WNT transgene expression via TagBFP (iWnt-SO), and receiver mouse embryonic stem cells that report β-catenin activity via eGFP. FIG. 11B shows live fluorescence microscopy of co-cultures of iWNT-SO-BFP and mouse embryonic stem cells with TCF/LEF-GFP reporters in the absence or presence of dox, for iWnt3a-SO cells (top) and iWNT9B-SO cells (bottom) with or without RSPO-1. Green signal is fluorescence indicates TCF/LEF-GFP reporter activation, blue signal indicates that cells express BFP-Wnt. Scale bar is 20 μm. FIG. 11C shows Day14 organoids coupled with iWnt3a-SOs at different doxycycline dosages, immunostained with antibodies for EMX2, POU3F3 and ZO-1 as indicated. Dashed white line is drawn around the organizer. Blue rectangle is magnified and shown in FIG. 11E. Scale bar is 200 μm. FIG. 11D shows radar plots showing distribution of POU3F3 and EMX2 fluorescence in nephrons relative to the nephron lumen (radar center) and organizer-adjacent point (blue dot) for nephrons shown in FIG. 11C. FIG. 11E shows a confocal close-up of iWnt3a-SO polarized nephrons (from blue dashed area in FIG. 11C), stained as show and with noted proximal-distal axis (two-arrowhead white dashed line). FIG. 11F shows organoids treated as in FIG. 11C immunostained with antibodies for DLL1, JAG1 and WT1 as indicated. Scale bar is 200 μm. FIGS. 11G-11H show graphs showing quantified nephron areas positive for DLL1 (FIG. 11G) and WT1 (FIG. 11H) per nephron. FIG. 11I shows Day14 organoids coupled with iWNT9B-SOs at different doxycycline and RSPO1 dosages, immunostained against DLL1, JAG1 and WT1 as indicated. Scale bar is 200 μm. FIGS. 11J-11L show graphs showing quantified nephron areas positive for DLL1 (FIG. 11J), JAG1 (FIG. 11K), and WT1 (FIG. 11L) per nephron as shown in FIG. 11I. See also FIGS. 10L-10N for statistical analysis.

FIGS. 12A-12O show organoid nephron proximal-distal patterning and polarization can be controlled by Wnt-secreting synthetic organizers. FIG. 12A shows 3D renders of Day18 organoids coupled with control synthetic organizers or Wnt3a synthetic organizers, immunostained for distal tubule markers POU3F3 and CDH1, and the tight-junction marker ZO-1. Scale bar is 100 μm. Bottom panels show top 30 POU3F3 objects descending by volume, for nephrons located under the organizer and towards the organoid's periphery (under-and-out, above dotted line). Scale bar is 100 μm. FIG. 12B shows percentages of nephron categories underneath organizers shown in FIG. 12A. FIG. 12C shows total POU3F3 segment volume from nephrons located in the under-and-out position. FIG. 12D shows distances between organizer peripheries and nearest renal corpuscles located towards the organoid's periphery (out) or center (in). FIG. 12E shows breakdown of total POU3F3 volumes from FIG. 12C, showing POU3F3 volume distributions relative to the distance from the organizer. FIG. 12F shows sample nephrons of the ‘short’ and ‘elongated’ morphologies as shown in FIG. 12A with purple boxed regions; and number of nephrons of the elongated morphologies in control vs. Wnt3a coupled organoids. FIG. 12G shows tubule morphoalignment concept indicating alignment of nephron PD axes to organizer and Wnt source. FIG. 12H shows immunostained organizers-coupled organoids with antibodies for ZO-1, POU3F3 and CDH1/3, annotated with the nephron PD axis (yellow dotted arrow), putative morphogenetic fields emanating radially from the center of the organizer (white arrow lines), alignment angle (yellow disc segment) and border of the organizer (dotted dark pink line). On the right, distribution of alignment angles of the nephrons PD axis relative to the morphogenetic field lines; indicated are p and Z values obtained from Rayleigh statistical test comparing measured distribution with uniform. Scale bar is 200 μm. FIG. 12I shows a dot plot of alignment angles of nephrons PD axis relative to the morphogenetic field lines, at increasing distance from the organizers. FIG. 12J shows 3D renders of TFAP2A, HNF4A, and WT1 structures in iWnt3a-SO and iWNT9B-SO coupled organoids in Day18 organoids at the indicated doxycycline dosages. Scale bar is 100 μm. FIGS. 12K-12L show distribution of TFAP2A and HNF4A volumes relative to the distance from the organizer for nephrons in the under-and-out position for iWnt3a (FIG. 12K) and iWNT9B (FIG. 12L) organizers. FIGS. 12M-12N show close-ups of nephrons surrounding iWnt3a-SO and iWNT9B-SO showing tubular alignment and patterning at basal, intermediate, and peak dosages. Scale bar is 50 μm FIG. 12O shows a proposed model: Constitutive provision of canonical WNT/β-catenin ligands at high levels distalizes developing nephrons at the expense of proximal identities. Tuning WNT levels induces distalization and morphoalignment, while minimizing suppression of proximal segments, yielding nephrons with continuous patterning.

FIGS. 13A-13E show distal nephron segments expand at the expense of proximal segments in Day 18 organoids coupled to constitutive WNT synthetic organizers. FIG. 13A shows 3D render snapshots of Day18 organoids coupled with control spheroids or constitutive Wnt3a organizers on Day12, immunostained against TFAP2A distal tubules, HNF4A proximal tubules and WT1 podocytes. Bottom panels show top 30 TFAP2A and HNF4A objects descending by volume, for nephrons located underneath the organizer and towards the organoid's periphery (out, above dotted line). Scale bar is 100 μm. FIGS. 13B-13C show distribution of TFAP2A (FIG. 13B) and HNF4A (FIG. 13C) volumes relative to the distance from the constitutive Wnt3a organizer in 10 μm increment bins for nephrons in the out region. In FIG. 13C, a threshold of 90 um is indicated based on when the HNF4A volumes transition from negligible to visible. FIGS. 13D-13E show total volumes (FIG. 13D) and number counts (FIG. 13E) of TFAP2A and HNF4A segments in day 18 organoids coupled with control or constitutive Wnt3a organizers, in nephrons in the out region.

FIGS. 14A-14J show activation of distal tubule development in Day 18 organoids coupled with iWNT synthetic organizers (in support to FIGS. 12A-12O). FIG. 14A shows top 30 3D-render TFAP2A and HNF4A structures in iWnt3a-SO-coupled day 18 organoids at the indicated doxycycline dosages sorted by descending volume. FIGS. 14B-14C show total volumes of TFAP2A (FIG. 14B) and HNF4A (FIG. 14C) segments in iWnt3a-SO coupled day 18 organoids at different doxycycline dosages in under-and-out nephrons. FIG. 14D shows distance of renal corpuscle structures in iWnt3a-SO coupled day 18 organoids at the indicated doxycycline dosages in under-and-in nephrons. FIG. 14E shows top 30 3D-render objects TFAP2A and HNF4A structures in iWNT9B-SO+100 ng/mL rhRSPO1 coupled day 18 organoids at the indicated doxycycline dosages sorted by descending volume. FIGS. 14F-14G show total volumes of TFAP2A (FIG. 14F) and HNF4A (FIG. 14G) segments in iWNT9B-SO+100 ng/mL rhRSPO1 coupled day 18 organoids at the indicated doxycycline dosages in under-and-out nephrons. FIG. 14H shows widefield immunostains of Day 18 organoids coupled with iWNT9B-SO+100 ng/mL rhRSPO1 at the indicated doxycycline dosages marking distal precursors (TFAP2A), proximal precursors (HNF4A), and podocyte precursors (WT1). FIG. 14I shows widefield immunostains of Day 18 organoids coupled with iWNT9B-SO+100 ng/mL rhRSPO1 at different doxycycline dosages marking connecting tubule precursors (GATA3), epithelial nephron precursors (HNF1B), and podocyte precursors (MAFB). FIG. 14J shows morphoalignment of nephron tubules in day 18 organoids coupled with iWNT9B-SO+100 ng/mL rhRSPO1 at the indicated doxycycline dosages and corresponding p and Z values. On the right, two representative fluorescent microscope images of samples at the indicated dox concentration are shown. Colors indicate: red is constitutive mCherry from organizer cells, blue is inducible BFP from organizer cells, and green is live reporter for proximal tubule marker HNF4A. FIGS. 14H-14I show fluorescent images from WNT9B-SO coupled organoids at day 18, stained and treated as indicated on figure—dashed line marks organizer, lower panel shows SO-distant contralateral area of organoid. Scale bars are 500 μm.

FIGS. 15A-15B show that kidney organoid nephrons form non-polarized and respond to cell-secreted localized WNT ligands. FIG. 15A shows UMAP plots showing organoid scRNAseq for days 10, 12, and 14, with lineage and differentiation markers shown as feature plots. FIG. 15B shows histograms comparing number of cells with expression of WNT4 and FGF8 between developing kidney and kidney organoid single-cell transcriptomic datasets.

FIGS. 16A-16H show activation of distal tubule development in Day18 organoids coupled with WNT synthetic organizers (in support to FIG. 4). FIG. 16A shows overview of single-cell RNA sequencing for nephrons near signaling centers following exclusion of mCherry+HEK-293 cells and selection of DAPI-DRAQ5+ live cells (n=22 organoids). UMAP shows segregated interstitium and nephron clusters; violin plots show sequencing quality control metrics (gene reads per cell, total reads per cell, mitochondrial content per cell) across annotated clusters. FIG. 16B shows expression of nephron progenitor (SIX1, CITED1), podocyte (MAFB, NPHS1, PODXL, WT1), proximal (HNF4A), distal (POU3F3, EMX2) and medial (JAG1, FGF8) precursor markers, interstitium-enriched genes (PDGFRA, PDGFRB), as well as kidney WNT ligands (WNT9B, WNT4) and downstream effectors (DKK1, AXIN2, LEF1) across annotated clusters. FIG. 16C shows expression of kidney (PAX8), interstitium (PDGFRB), podocyte (NPHS1), proximal precursor (HNF4A) and distal precursor (POU3F3) markers in the UMAP. FIG. 16D shows an overlay plot of origin UMAP showing whether data points originate from control or +cWnt3a organizer-conjugated samples, and further analysis within the nephron clusters. FIG. 16E shows quality control metrics within subset nephron-only clusters. FIG. 16F shows expression of β-catenin targets, podocyte, proximal, medial, and distal lineage marker expression across nephron-only clusters. FIG. 16G shows UMAP plots showing podocyte, proximal tubule, and distal tubule populations based on segregated expression of WT1, HNF4A, and POU3F3 respectively within nephron-only UMAPs. FIG. 16H shows percentage of transcript reads coming from WNT-vs-control-conjugated organoids for indicated genes, for the total sum of reads for each gene.

FIGS. 17A-17B show organoid nephron proximal-distal patterning and polarization controlled by synthetic WNT signaling centers. FIG. 17A shows tubule morphoalignment concept (left) and immunostained examples (right) verifying that tubular morphologies oriented towards WNT organizers are distal. FIG. 17B shows the number of tubule path intersections relative to the number of paths drawn per organoid.

FIGS. 18A-18F shows morphoalignment and axial patterning in organoid nephrons enabled by WNT secreting organizers (in support to FIG. 4). FIG. 18A shows an example of tubular morphoalignment quantification in Day18 HNF4A:YFP proximal reporter organoids coupled with control or constitutive Wnt3a organizers. The dot marks the organizer center; arrows mark proximal HNF4A:YFP+ tubules that extend into non-proximal HNF4A:YFP− tubules; lines show their projected proximal-to-distal trajectory paths that intersect at variable frequencies and distances from the organizer center. FIG. 18B shows distance between tubule path intersections and the organizer's center per organoid. FIG. 18C shows top 30 TFAP2A and HNF4A nephron objects descending by volume for nephrons located in the under-and-out position, from 3D renders of Day18 organoids coupled with iWnt3a organizers at basal, intermediate, and peak dox dosages. FIGS. 18D-18E show total TFAP2A (as in FIG. 18D) and HNF4A (as in FIG. 18E) volumes for nephrons in the under-and-out position in iWnt3a-coupled Day18 organoids. FIG. 18F shows distances between organizer peripheries and nearest renal corpuscles located towards the organoid's periphery (out) or center (in), for iWnt3a-coupled Day18 organoids at different dox dosages.

FIG. 19A and FIGS. 19B1-19B9 show human developing kidney single-cell RNA sequencing analyses. FIG. 19A shows expression of the collecting duct stalk and tip markers, and genes enriched in self-renewing nephron progenitors, differentiating nephron progenitors, distal precursors, proximal precursors, and podocyte precursors. FIGS. 19B1-19B9 show ligand-receptor pairing analyses using CellChat to predict cell signaling in the early collecting duct and nephron lineage.

FIGS. 20A-20I show the use of pluripotent stem cells and iUBs as synthetic organizers. FIG. 20A shows an overview of genotyping strategy for CRISPR-mediated knock-in of dox-inducible WNT transgenes in the CLYBL locus. FIGS. 20B-20C show genotyping for the intact CLYBL locus (FIG. 20B) and site-specific integration of inducible WNT circuits in a panel of H1 clones edited with Wnt3a/WNT9B circuits alongside parental H1 embryonic stem cells (FIG. 20C). FIG. 20D shows constitutive expression of mCherry-reported transactivator cassette and inducibility of BFP-reported WNT cassette in heterogeneous populations of transgenic Wnt3a/WNT9B edited H1 cells. FIG. 20E shows a panel of transgenic H1 clones edited with Wnt3a/WNT9B circuits demonstrating stable mCherry expression 6 passages after clone derivation. FIG. 20F shows co-cultures between 7xTCF/LEF::eGFP reporter mouse embryonic stem cells and H1 human embryonic stem cells engineered with Wnt3a/WNT9B circuits. FIG. 20G shows H1-derived ureteric organoids infected with iWnt3a/iWNT9B lentiviruses showing constitutive mCherry-marked transactivator expression and inducible BFP-marked WNT expression. FIG. 20H shows localized nephrogenesis and tubular morphoattraction (yellow arrows) in kidney organoids co-cultured with inducible Wnt3a ureteric organoids. FIG. 20I shows immunostains of iWnt3a iUBs (magenta) coupled to kidney organoids showing morphoattraction and fusion of connecting tubules (yellow) in continuum to distal tubules (red) of kidney organoid nephrons.

### DETAILED DESCRIPTION

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this disclosure belongs. The term “comprising”, and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

The following definitions are provided for the full understanding of terms used in this specification.

### Terminology

The term “about” as used herein when referring to a measurable value such as an amount, a percentage, and the like, is meant to encompass variations of ±20%, ±10%, ±5%, or ±1% from the measurable value.

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units is also disclosed. For example, if 10 and 15 are disclosed, then 11, 12, 13, and 14 are also disclosed.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

As used herein, “codon” refers to the genetic code used by living cells to translate information encoded by genetic material (DNA or mRNA sequences of nucleotide triplets) into protein. This term also refers to the genetic code that specifies which amino acids will be added next during protein synthesis.

A “control” is an alternative subject or sample used in an experiment for comparison purposes. A control can be “positive” or “negative.”

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom, Thus, a gene encodes a protein if transcription and translation of mRNA occurs.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

The term “increased” or “increase” as used herein generally means an increase by a statically significant amount; for the avoidance of any doubt, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level so long as the increase is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

As used herein, the terms “may,” “optionally,” and “may optionally” are used interchangeably and are meant to include cases in which the condition occurs as well as cases in which the condition does not occur.

The term “nucleic acid” as used herein means a polymer composed of nucleotides, e.g., deoxyribonucleotides or ribonucleotides.

The term “nucleobase” refers to the part of a nucleotide that bears the Watson/Crick base-pairing functionality. The most common naturally-occurring nucleobases, adenine (A), guanine (G), uracil (U), cytosine (C), and thymine (T) bear the hydrogen-bonding functionality that binds one nucleic acid strand to another in a sequence specific manner.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

A “promoter,” as used herein, refers to a sequence in DNA that mediates the initiation of transcription by an RNApolymerase. Transcriptional promoters may comprise one or more of a number of different sequence elements as follows: 1) sequence elements present at the site of transcription initiation; 2) sequence elements present upstream of the transcription initiation site and; 3) sequence elements down-stream of the transcription initiation site. The individual sequence elements function as sites on the DNA, where RNA polymerases and transcription factors that facilitate positioning of RNA polymerases on the DNA bind.

As used throughout, by a “subject” (or a “host”) is meant an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) mammals, non-human mammals, primates, non-human primates, rodents, birds, reptiles, amphibians, fish, and any other animal. The subject can be a mammal such as a primate or a human. Administration of the therapeutic agents can be carried out at dosages and for periods of time effective for treatment of a subject.

Disclosed herein are the components to be used to prepare the disclosed compositions as to be used in the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. If a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

As used herein, the term “vector” refers to any moiety which can deliver a nucleic acid sequence into a cell or virus so that the nucleic acid sequence can be replicated and/or expressed by the cell or virus. In some aspects, the vector can be any suitable in vivo gene expression vector. In some aspects, the vector can be a viral vector. For example, in some aspects, the vector can be an adenovirus vector. As used herein, the term “adenovirus,” abbreviated “Ad,” refers to viruses of the adenoviridae family. Adenovirus is a medium-sized (90-100 nm), nonenveloped icosahedral virus containing double-stranded DNA. The term “adenoviridae” refers collectively to adenoviruses of the genera Atadenovirus, Aviadenovirus, Ichtadenovirus, Mastadenovirus, and Siadenovirus. “Adenovirus” includes, but is not limited to human, bovine, ovine, equine, canine, porcine, murine and simian adenovirus species. Human adenoviruses, i.e., adenoviruses that can infect humans, can be classified into subgenera, or species, A-G. Similarly, the term “adenovirus vector” or “adenoviral vector” refers to an adenovirus containing, in the genome thereof, a sequence other than the intrinsic base sequence of the adenovirus, for example any of the disclosed nucleic acids.

In other such aspects, the vector can be a retroviral vector. The term “retroviral vector” refers to a vector containing structural and functional genetic elements that are primarily derived from a retrovirus.

In yet other such aspects, the vector can be a lentiviral vector. The term “lentiviral vector” refers to a vector including one or more nucleic acid sequences derived from at least a portion of a lentivirus genome.

In some aspects, the vector can be a plasmid. The term “plasmid” refers to an extra chromosomal element often carrying a gene that is not part of the central metabolism of the cell, for example any of the disclosed nucleic acids, and usually in the form of circular double-stranded DNA molecules.

In some aspects, the vector can be a cosmid. In some aspects, the vector can be an artificial chromosome. In another aspect, provided is a vehicle (e.g., lipid nanoparticle) including any of the disclosed vectors.

### Synthetic Organizers, Systems, and Methods

In some aspects, disclosed herein is a synthetic organizer comprising one or more cells, wherein the one or more cells comprise a nucleic acid construct comprising a WNT3A gene or a WNT9B gene under the control of an inducible promoter. In some embodiments, the synthetic organizers are self-organizing WNT secreting cells.

The WNT family of genes produce glycolipoproteins that are involved with signaling and developmental processes. WNT3A, one of WNT family members, plays key roles in regulating pleiotropic cellular functions, including self-renewal, proliferation, differentiation, and motility. WNT9B gene, another Wnt family member, encodes for the WNT9B protein which participates in the canonical WNT/β-catenin signaling pathway. WNT9B functions in the establishment of the kidneys, because WNT9 is critical for morphogenesis of the nephron.

An inducible promoter is a regulatory component that allows for the control of gene expression in response to a specific stimulus. The specific stimulus causes them to bind to RNA polymerase and transcriptional factors. This initiates the transcription process. In some embodiments, the inducible promoter is a Tet promoter. In some embodiments, the inducible promoter can be selected from a group comprising a tetracycline-inducible promoter, a heat shock inducible promoter (for example, Hsp70 or Hsp90-derived promoters), or a dexamethasone inducible promoter. Other examples include: Grazoprevir (GZV)-inducible promoters, tamoxifen (40HT/TMX)-inducible promoters and abscisic acid (ABA)-inducible promoter.

In some embodiments, the one or more cells comprise kidney cells. In some embodiments, the kidney cells comprise human embryonic kidney (HEK) cells, collecting duct cells, or iPS-derived ureteric bud precursor cells. In some embodiments, the kidney cells comprise an induced pluripotent stem cell (iPSC). Current iPSC-derived kidney organoids contain nephron-like structures, but these lack physiological patterning and tubular organization. Nephrons are thought to form their proximal-distal axis in response to collecting duct-derived WNT ligands. Kidney organoids lack collecting ducts and associated WNTs, which might be responsible for the absence of nephron patterning.

In some embodiments, the one or more cells comprise pluripotent stem cells. In some embodiments, the pluripotent stem cells include embryonic stem cells, mesenchymal stem cells, or induced pluripotent stem cells.

In some embodiments, the one or more cells comprise a ureteric organoid (iUB).

In some embodiments, the one or more cells are human.

In some embodiments, the kidney cell comprises an additional nucleic acid construct comprising a CDH3 (encodes E-cadherin) or CDH1 (encodes P-cadherin) gene. In some embodiments, the additional nucleic acid construct comprises CDH2 (encodes N-cadherin), CDH4 (encodes R-cadherin), CDH6 (encodes K-cadherin), or CDH15 (encodes M-cadherin). CDHs are genes that encode cadherins, which are adhesion molecules that help maintain cell architecture. It could also use other adhesion proteins like protocadherins, Selectins, Teneurins and latrophilins. It could also comprise synthetic adhesions like GFP/anti-GFP synCAMs. These proteins are also adhesion proteins that help maintain cell architecture.

In some embodiments, the nucleic acid construct and/or the additional nucleic acid construct are provided to the one or more cells as a vector. Examples of vectors are described in detail above.

In some embodiments, the nucleic acid construct and/or the additional nucleic acid construct are transfected into the one or more cells. In some embodiments, the nucleic acid construct and/or the additional nucleic acid construct are inserted into the genome of the cell (for example, using CRISPR to knock-in the nucleic acid construct and/or the additional nucleic acid construct at a particular locus of the genome of the one or more cells).

In some aspects, also disclosed herein is a system comprising a synthetic organizer and a human kidney organoid nephron, wherein the synthetic organizer facilitates patterning of the human kidney organoid nephron. In some embodiments, the synthetic organizer can include any of the synthetic organizers described above.

In some aspects, also disclosed herein is a method of patterning a human kidney organoid nephron, comprising: obtaining a human kidney organoid nephron; and contacting the human kidney organoid nephron with a synthetic organizer. In some embodiments, the synthetic organizer can include any of the synthetic organizers described above.

In some embodiments, the method further comprises a step of detecting expression of a distal lineage gene marker, wherein the expression of the distal lineage gene marker signals the presence of distal nephron cells. In some embodiments, the distal tubule markers can be LGR5, POU3F3, and/or TFAP2A. In some embodiments, distal tubule markers can be Calb1, UMOD, SLC12A1, SLC12A3, AP-2α and AP-2β, E-Cadherin, P-Cadherin, K-Cadherin N-Cadherin, R-Cadherin, M-Cadherin, Jag1, Wt1, Pax2, Pou3f3, and/or Dll1.

### EXAMPLES

The following examples are set forth below to illustrate the compositions, cells, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: Patterning Human Kidney Organoids with Synthetic Wnt-Secreting Organizers

Kidney organoids generated from human induced pluripotent stem cells (hiPSCs) are a promising source of synthetic kidney tissue to solve the scarcity of donor kidneys available for transplantation. To reach clinical relevance, organoids must at minimum generate nephrons that reproducibly pattern along their proximal-distal (PD) axial polarity and form proximal renal corpuscles capable of filtering blood, proximal convoluted tubules controlling the majority of solute reabsorption, and distal connecting tubules that facilitate draining of urine into a collecting duct. Nephrons in current models display limited cell maturation and incomplete PD patterning, likely due to absent signaling cues that are present in vivo.

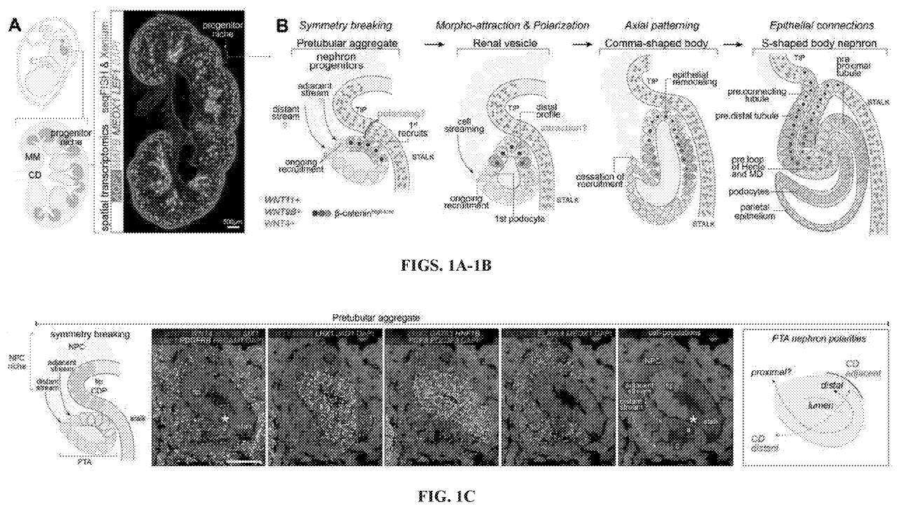

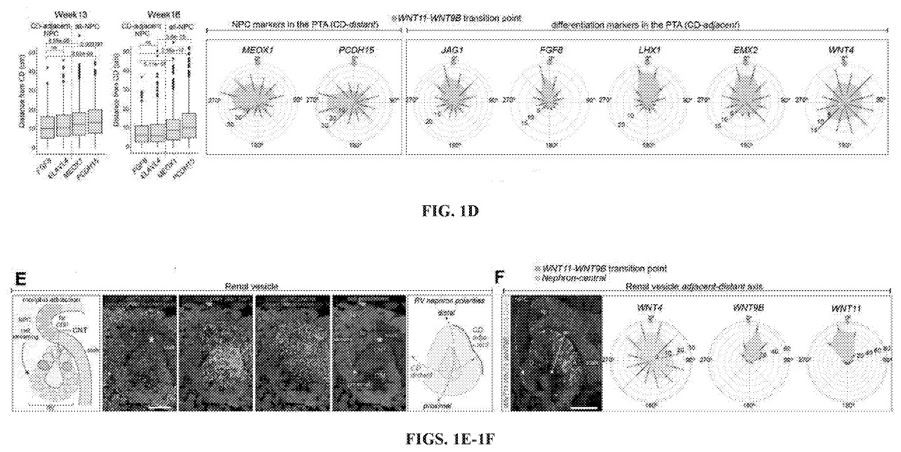

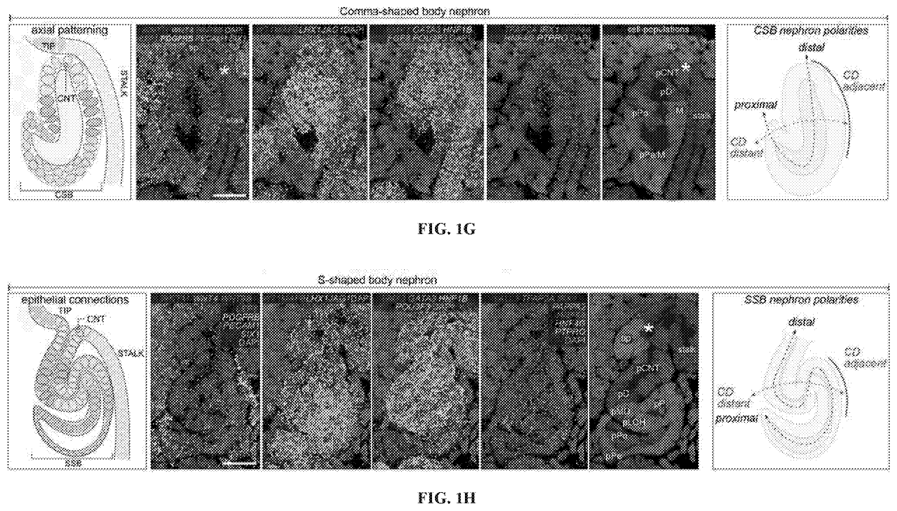

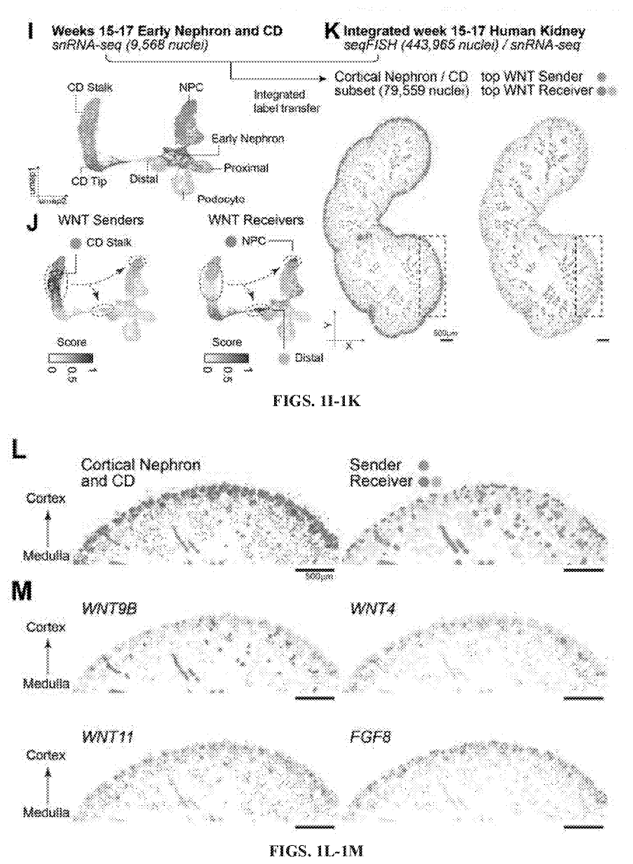

Nephrons form during kidney organogenesis from reciprocal interactions between the collecting duct (CD), nephron progenitor cells (NPCs), and stromal progenitors. Throughout kidney development the forming nephron and CD develop concurrently and in contiguity (FIG. 1A). The developing CD includes molecularly distinct tip progenitors, marked by expression of the secreted signaling protein Wnt family member 11 (WNT11), and their progeny stalk cells in which WNT9B is expressed. Layers of NPCs surround CD tips and balance self-renewal and differentiation in response to stalk-derived WNT9B ligands that activate β-catenin signaling and initiate nephrogenesis. The first sign of nephron formation is a stream of NPCs that exit their CD tip niche, progressively decrease progenitor markers, and gradually condense into a pretubular aggregate (PTA) under the CD tip. At this point, the PTA epithelializes and grows into a lumenized renal vesicle (RV) displaying the first signs of polarization with regionalized gene expression in the distal domain adjacent to the CD stalk. This early asymmetry becomes further emphasized as the RV develops into Comma- and then S-shaped body nephrons (CSB and SSB); the latter when the PD axis can be broadly defined by distinct gene expression in distal, medial, proximal, and podocyte domains. Studies in mice have shown that distal cells in the early nephron display high β-catenin activity and increasing Wnt/β-catenin signaling leads to an expansion of distal segments at the expense of medial and proximal ones. Given the spatial relationship between the forming nephron and the CD, and Wnt and β-catenin components controlling distal nephron development and nephrogenesis, it has been hypothesized that the Wnt-expressing CD serves as a signaling organizer for the early nephron, setting up a morphogenetic field that initiates symmetry breaking and PD axial patterning. Whether this holds true in the developing human kidney remains unknown.

In efforts to generate kidney tissue from pluripotent stem cells, several kidney organoid models have been described that aim to replicate the developmental progression from stem cells to mesoderm, posterior intermediate mesoderm, and subsequent nephron differentiation. These organoid models typically lack a CD epithelium and develop nephrons whose patterning along the PD axis is incomplete. Since CD epithelium can be derived from an anterior intermediate mesoderm lineage through separate differentiation protocols, CD and NPCs populations have been mixed with the result that a higher degree of self-organization and patterning occurs. These self-organizing human models represent a promising step forward. However, they generate only about 20 connected nephrons, their PD domains remain non-proportional and incompletely patterned compared to in vivo counterparts, and their organization lacks extrinsic control. Determining how patterns form in vivo and whether new mechanisms can be applied to generate accurate axial PD nephron patterning is therefore an important goal. Methods to control morphogenetic fields in organoids have been recently proposed and include synthetic approaches where patterning cues are provided either via microfluidics delivery or from engineered cells. In particular, cell-based signaling centers have been used to pattern embryoid bodies derived from mouse embryonic stem cells and in human induced pluripotent stem cells (iPSC)-derived brain organoids. Thus, growth factors from adjacent cell sources can initiate and locally pattern regions of stem-cell-derived constructs.

Here a study was conducted which tested whether recapitulating the CD-to-nephron signaling relationship in human kidney organoids could provide a means to control and normalize organoid nephron patterning. The study resolved the spatial transcriptional patterns of the developing human kidney and showed that nephrons develop their axial polarities adjacent to a transition point in the CD between CD precursors (expressing WNT11) and CD stalk cells (expressing WNT9B). Nephron cells adjacent to the transition point express canonical β-catenin-dependent WNT targets, form a distal nephron identity, and ultimately connect to the CD epithelium to develop a luminal connection that enables drainage of urine into the CD. This detailed nephron transcriptional profile demonstrates that the process of nephrogenesis occurs through an inherently asymmetric mechanism: nephrons first develop a CD-adjacent to CD-distant (AD) axial polarity that presages formation of its PD axial polarity and later nephron anatomies. Comparative analyses between kidneys and organoids revealed that organoids do not display organized activation of canonical WNT signal inputs. Coupling organoid nephrons with WNT-secreting synthetic organizers drove a canonical WNT response, promoted distal nephron differentiation programs, and generated a morphogenetic response in nephrons, prompting them to orient their elongating axes towards the WNT source in a process occurring over several days. The data highlight a strategy to engineer axial asymmetry and patterning in kidney organoids and to exert spatial control over differentiation programs in stem-cell-derived organoids in general.

### Results

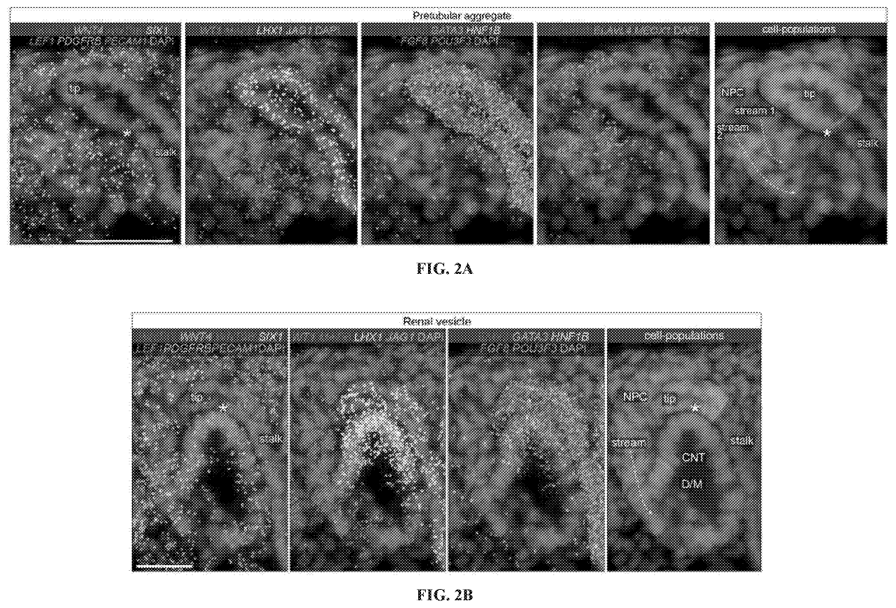

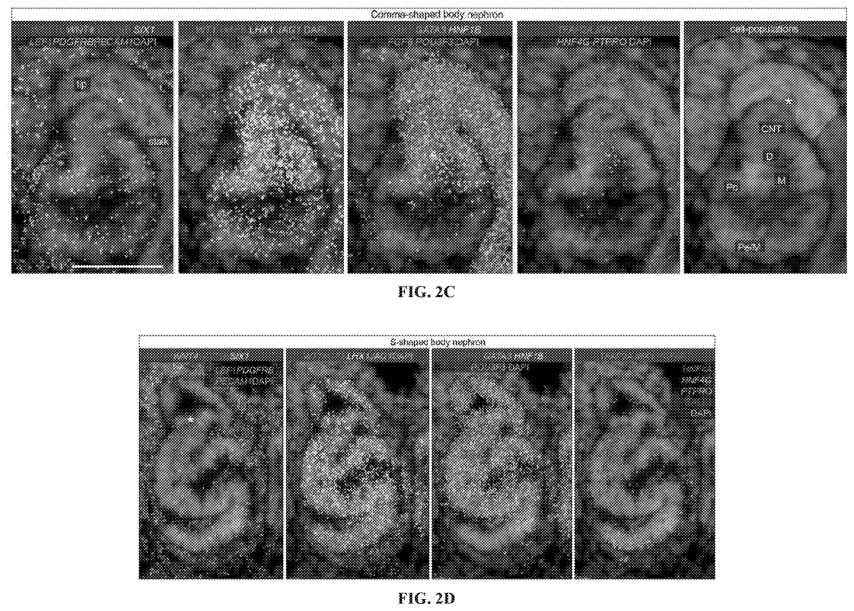

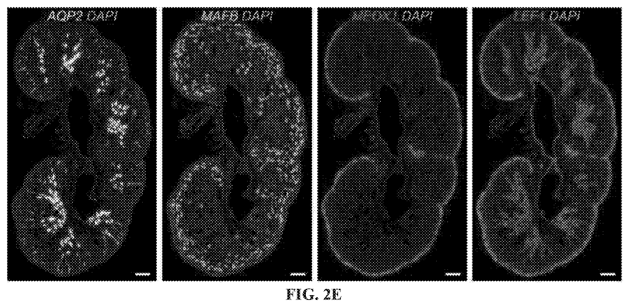

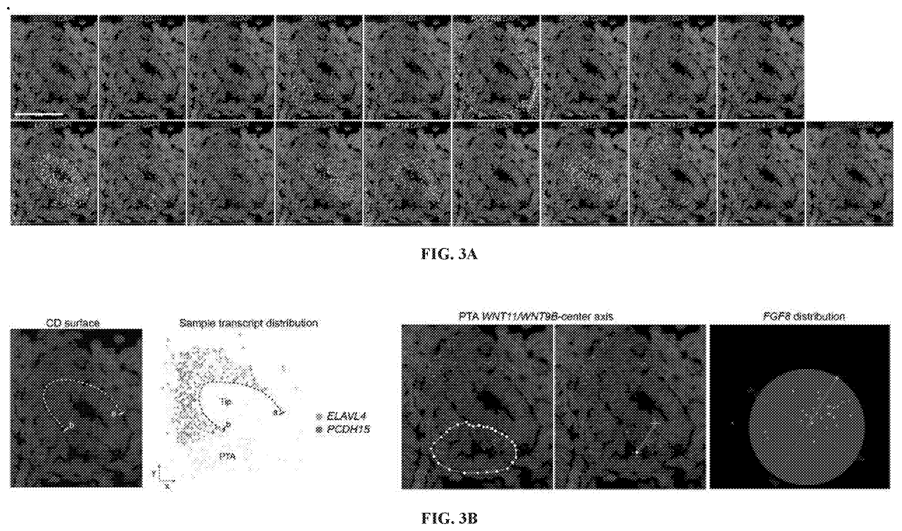

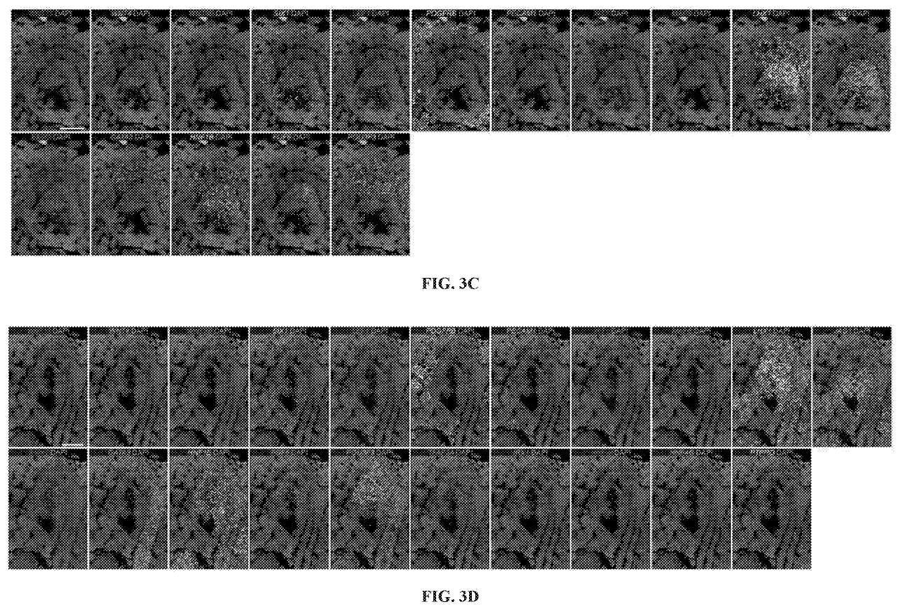

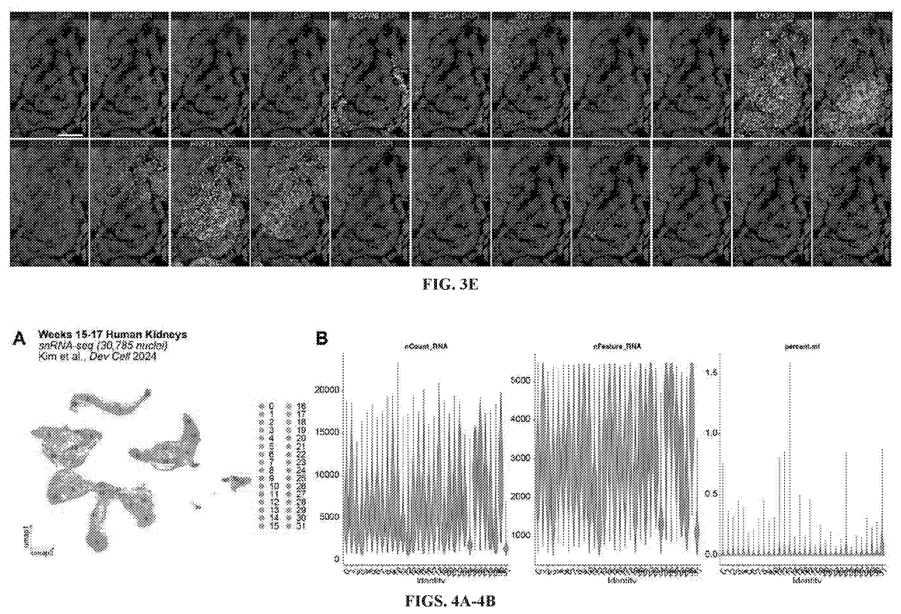

Cross-lineage communication defines nephron polarization and morphogenesis in the human developing kidney: To scrutinize signaling interactions within the forming human nephron and its niche, the study performed spatial transcriptional analyses on human week 13 and week 16 developing kidneys using two spatial transcriptomic platforms: a hybrid approach combining in situ sequencing (ISS) and in situ hybridization (ISH) (Xenium) and a sequential Fluorescence In Situ Hybridization (seqFISH). These allowed mapping of the expression of a panel of ~250 genes across all stages of early nephrogenesis, given that nephrons develop asynchronously and continuously across the kidney cortex (FIG. 1, FIG. 2, FIG. 3). The genes were selected to resolve nephron patterning and kidney organogenesis. The study analyzed the expression of known and putative WNT target genes in relationship to WNT ligands and markers of cell types present in the developing kidney (FIG. 1A). These observations, described below, allowed determination of whether the WNT-expressing boundary in the CD could function as a de facto organizer for the forming nephron as it aggregates and forms an axial polarity (FIG. 1B).

At the first stage of nephrogenesis, two layers of mesenchymal NPCs leave their niche at the top of the CD tips and form a continuous stream of cells between their niche and morphologically recognizable ~75 μm wide PTA cell aggregates just below the tips that display asymmetric cell polarization and partial lumen formation towards the CD (FIG. 1C). At this stage, aligned to the NPC to PTA transition, there is an anatomically matching sharp boundary in the CD between WNT11 expressing (non-canonical WNT) CD tip progenitors and WNT9B expressing (canonical WNT) CD stalk domains; this transition is highlighted with an asterisk in FIG. 1C.

To understand the cell states leading up to and forming the PTA asymmetry, the study performed quantitative analysis on the spatial distribution of transcripts in the seqFISH dataset. With these the study tested whether differences in gene expression can be observed in cells adjacent and distant to the CD tip-stalk boundary. In NPCs in their niche, a subset of genes was detected at higher frequency adjacent to the CD (Fibroblast Growth Factor 8—FGF8 and ELAV Like RNA Binding Protein 4—ELAV4). Others, such as Mesenchyme homeobox 1 (MEOX1) and Protocadherin 15 (PCDH15) had a broader distribution (FIG. 1C, FIG. 2A, FIGS. 3A-3B). This was true in both week 13 and week 16 samples. Inside the PTA, this asymmetric distribution of transcripts became pronounced. Known and putative Wnt targets in the mouse kidney (FGF8, LIM homeobox 1 (LHX1), Jagged Canonical Notch Ligand 1 (JAG1), Empty Spiracles Homeobox 2 (EMX2), and WNT4, were biased towards the WNT11-WNT9B transition zone in the CD. In contrast, NPC markers such as MEOX1 and PCDH1510,11 were more abundant in cells further from the CD (FIG. 1D). These observations support a scenario where gene expression asymmetry is intrinsic to the epithelializing nephron as determined by a ‘CD adjacent’ to ‘CD distant’ axis forming against the WNT11-to-WNT9B boundary in the CD.

The asymmetry seen in the PTA is observed and elaborated on in the subsequent developmental stage, the renal vesicle (RV). The study identified RVs as epithelial pear-shaped cysts ~100 μm long (FIG. 1E) and observed that the NPCs in their niche appear as a single line of cells that connects with the RV at the CD-distant end (FIG. 1B, FIGS. 1E-1F, FIG. 2B, FIG. 3C). The RV domain presented a distal gene expression profile in the direction of the CD WNT11-to-WNT9B transition point (marked with an asterisk in FIG. 1E). The transcripts that the study identified as asymmetrically distributed at the PTA stage remained asymmetrically distributed and extended along the PD axis.

Genes such as JAG1, EMX2, and HNF1 homeobox B (HNF1B) occupied larger domains, whereas FGF8 and WNT4 remained biased towards cells closer to the CD. A more defined distal domain emerged near the WNT11-to-WNT9B transition, marked by POU class 3 homeobox 3—(POU3F3) and rare GATA binding protein 3 (GATA3). At the proximal end of the RV, distant from the CD, some of the genes that were broadly distributed in the PTA stage e.g., Wilms' Tumor Protein 1 (WT1), take a polarized proximal expression distribution (FIG. 1E, FIG. 3).

To independently define and quantify the RV's transcriptional asymmetry, the study mapped WNT/β-catenin target WNT4 in the RV relative to the CD WNT11-to-WNT9B tip-stalk transition point using RNA scope in situ hybridization and immunolabeled structural protein Keratin 8 (KRT8), which marks the CD, the distal nephron, and defines cell boundaries. WNT4 transcripts were asymmetrically distributed towards the WNT9B expressing CD stalk (FIG. 1F), confirming the existence of a dynamic CD distant-adjacent axis in the RV. The initial asymmetries of the PTA therefore first define nephron patterning from the CD WNT11-to-WNT9B transition point, leading to an emerging connecting tubule (CNT) profile expressing GATA3 in the RV (FIG. 1E, FIG. 2B, FIG. 3C).

Given the potential impact of the CD adjacent-distant axis for describing nephron asymmetries and morphogenesis, the study investigated how the CD adjacent-distant axis and the PD axis co-develop after the RV stage through CSB and SSB stages (FIG. 1B, FIG. 1G, FIG. 2C, FIG. 3D). The CSB nephron elongates along its PD axis and cells nearest the CD WNT11-to-WNT9B transition point form CNT precursors positive for GATA3, POU3F3, and Transcription Factor AP-2 Alpha (TFAP2A), while the FGF8 domain expands and WNT4 shifts proximally along CD-adjacent cells. In the medial (now the middle of the PD axis) and proximal regions (here referring to podocytes), genes with specific sub-domain expression can be identified e.g., Iroquois homeobox 1 (IRX1) and MAF BZIP Transcription Factor B (MAFB), respectively (FIG. 1G, FIG. 2C, FIG. 3D). As the nephron transitions to the SSB (FIG. 1B), it adopts new morphological features of convolution, particularly the deep glomerular cleft in the CD-distant proximal domain, separation of the distal and medial domains, and the CNT forming a patent luminal connection with the CD (FIG. 1H, FIG. 2D, FIG. 3E). Signatures of precursor populations emerged. These included the connecting tubule (pCNT—GATA3), distal tubule (pD—TFAP2A, POU3F3, and, Spalt Like Transcription Factor 3 (SALL3)), loop of Henle and macula densa (pLOH and pMD—IRX1 and Pappalysin 2 (PAPPA2)), proximal tubule (pPo—HNF4A and HNF4G), parietal epithelium (pPe—WT1), and podocytes (pPo—WT1, MAFB, and Protein Tyrosine Phosphatase Receptor Type O—(PTPRO)) (genes names summarized in Methods for spatial transcriptomics). CD adjacent-distant asymmetric expression remains evident, for instance WNT4 being re-expressed in proximal precursors adjacent to the WNT9B expressing CD stalk (FIG. 1H, FIG. 3E). Overall, these spatial transcriptomic analyses reveal the stepwise patterning of the developing human nephron as a further elaboration of asymmetries generated close to the CD WNT11-to-WNT9B transition boundary.

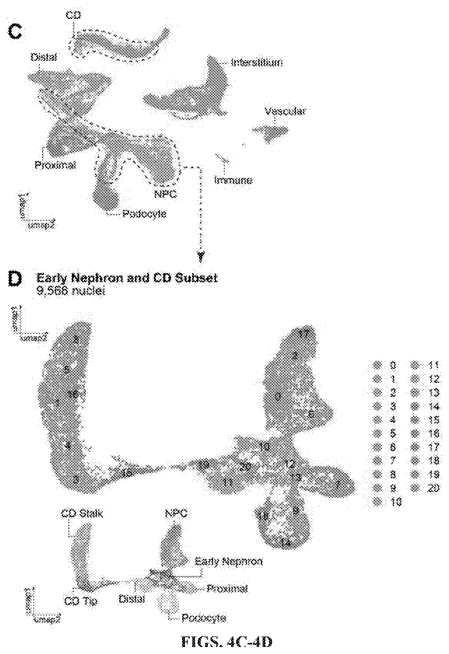

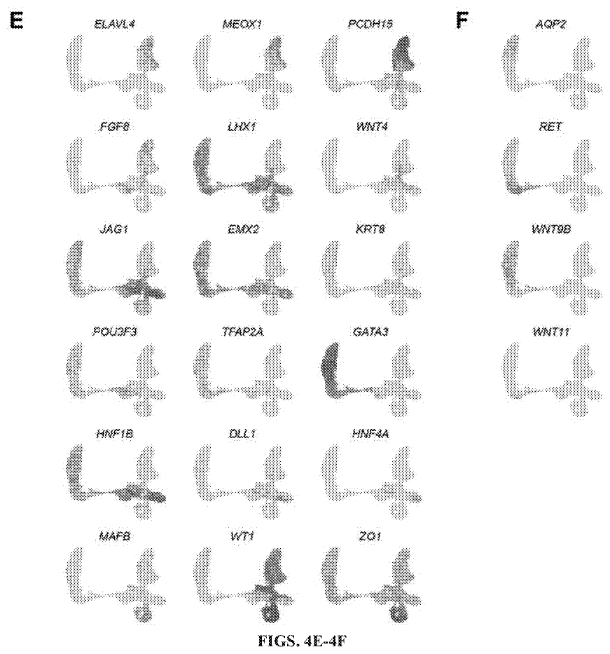

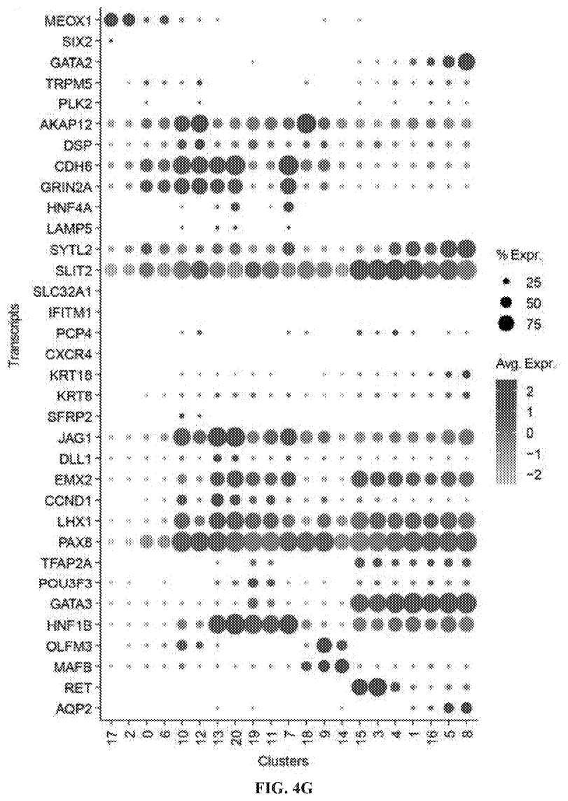

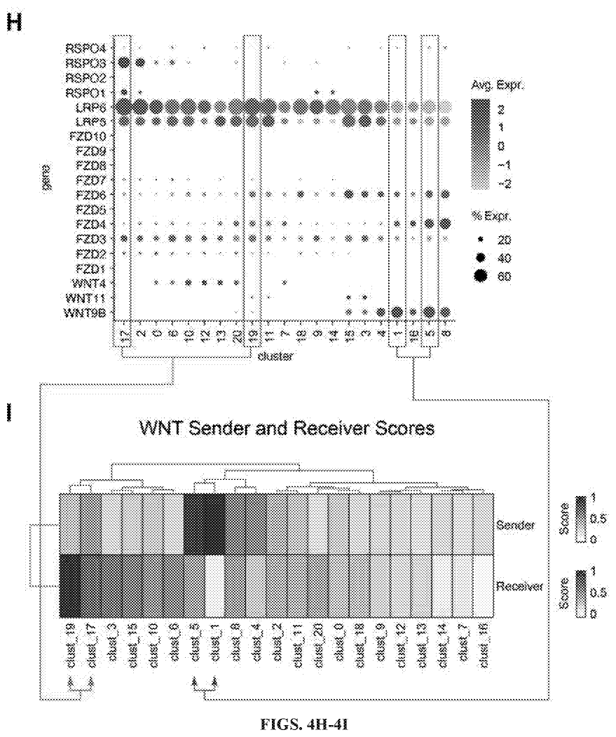

To globally test the possibility of canonical WNT signaling between WNT9B expressing CD cells and the CD-adjacent nephron, the study scrutinized single-cell (scRNA-seq, week 14 and 17) and single-nucleus sequencing data (snRNA-seq, week 15 to 17) for ligand-receptor interactions. These temporally match the spatial transcriptomic data presented above. The study resolved cell-types by iterative clustering and identified cortical CD tip progenitors and stalk expressing WNT11 and WNT9B respectively, and in the nephron progenitor lineages, NPCs and their early progeny (PTA/RV stages) as well as progenitors of distal, proximal, and podocyte cells (FIGS. 1I-1J, FIG. 4). The study explored potential cell signaling via CellChat, a computational tool that queries clustered single cell transcriptomic data and identifies putative ligand-receptor interactions between pairs of clusters (FIG. 1J, FIG. 4). The WNT9B expressing CD stalk was predicted to signal to NPCs, to PTA cells co-expressing WNT4 and FGF8, and to the early distal nephron.

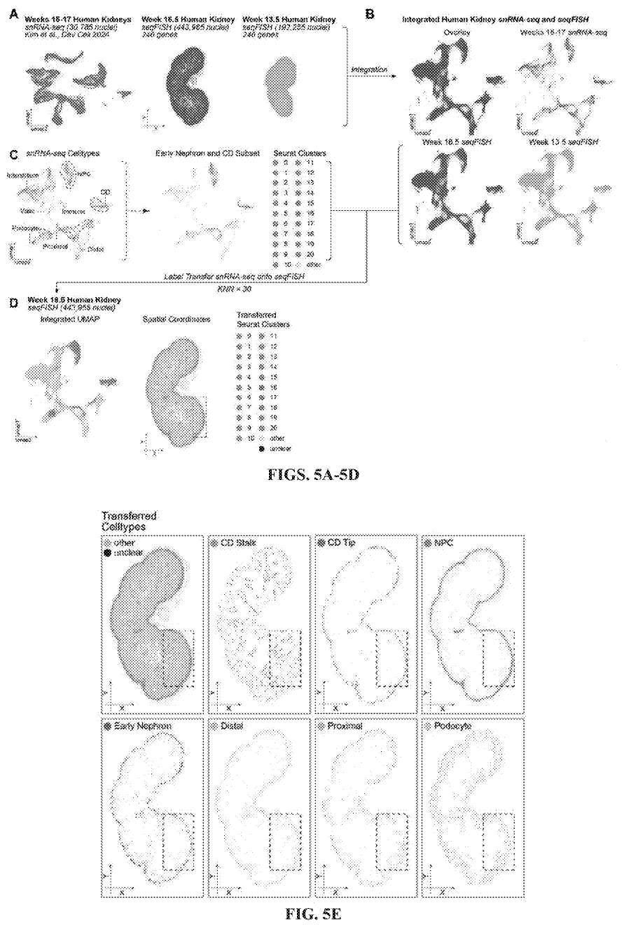

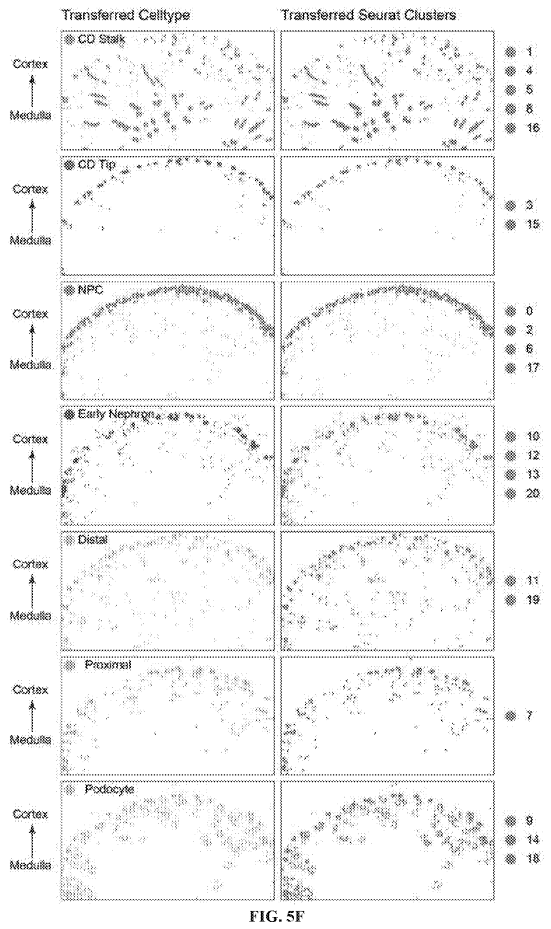

To contextualize this in the kidney anatomy, the study integrated spatial and snRNA-seq cell profiles based on transcriptional proximity between cell types identified in the spatial and in the snRNA-seq. Cortical CD and nephron identities mapped to cortical spatial coordinates and matched predicted cell-types and known anatomical relationships (FIG. 1K, FIG. 5, FIGS. 4D-4I). Predicted WNT-senders mapped to CD stalks (expressing WNT9B) adjacent to WNT4 and FGF8 expressing nephron cells (FIGS. 1L-1M). Among the two main predicted WNT-receiver populations, NPCs and distal nephron, the NPC expressed Frizzled 2, 3, 4, 6, 7 (FZD2, 3, 4, 6, 7) and Low-density Lipoprotein Receptor-Related Protein 5 and 6 (LRP5, 6), as well as R-spondin 1, 3, 4 (RSPO1, 3, 4). The distal nephron WNT-receivers were positive for the same FZD and LRP transcripts but were negative for all RSPO family members. The forming PTA where WNT4 and FGF8 are first detected also have no or significantly downregulated expression of RSPO1 and RSPO3. These data show that RSPO family members are differentially expressed in the developing nephron lineage, and are consistent with the notion, supported by mouse genetics, that Rspo1 and 3 are required for NPC maintenance and normal expression of Wnt4 but are downregulated in the forming nephron. In mice, Rspo1 and Rspo3 are specifically restricted to NPCs and the early nephron, thus matching the predicted WNT sender-receiver relationship in the cortical nephron forming niche (FIGS. 1L-1M).

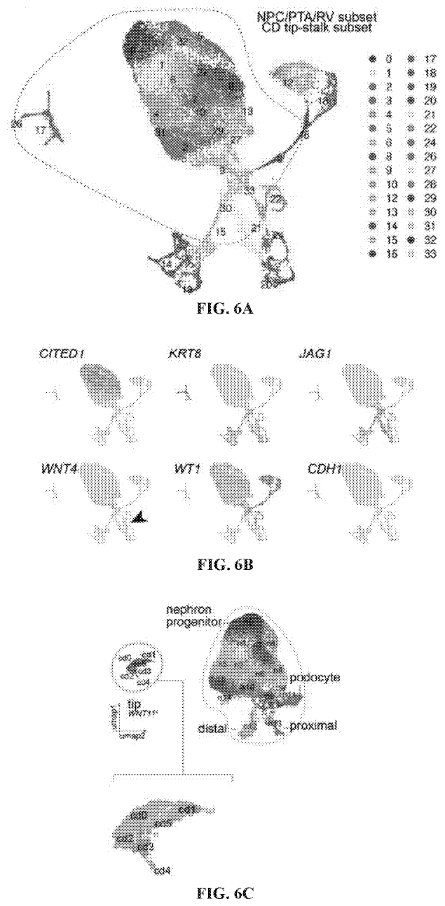

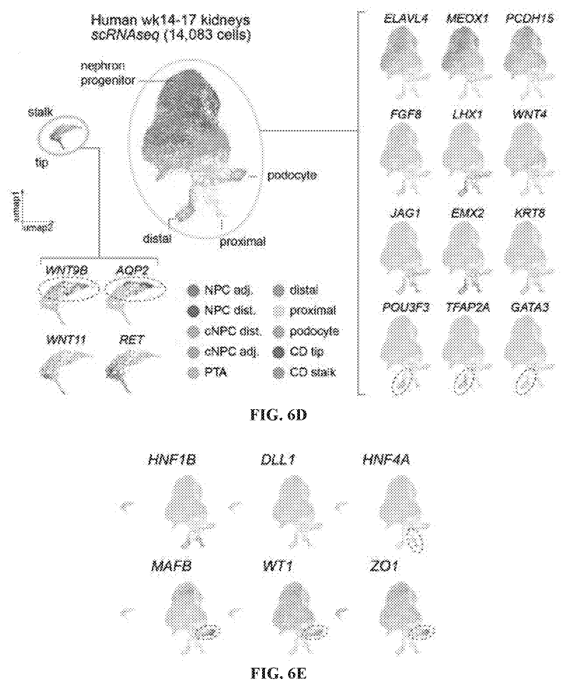

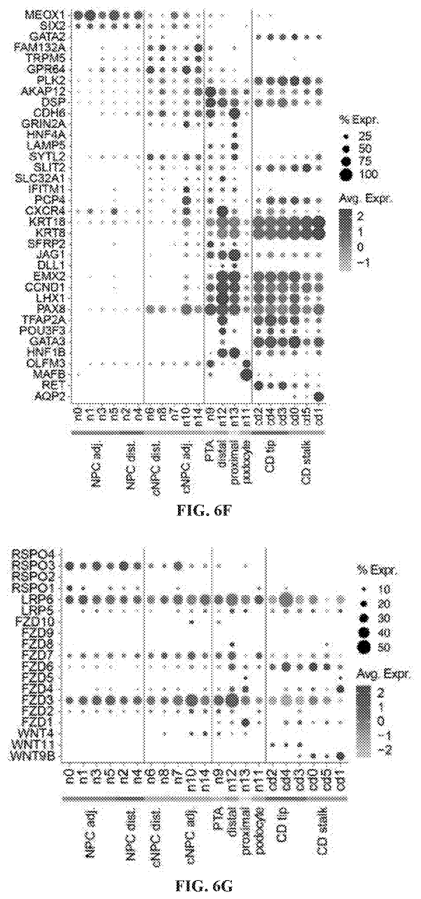

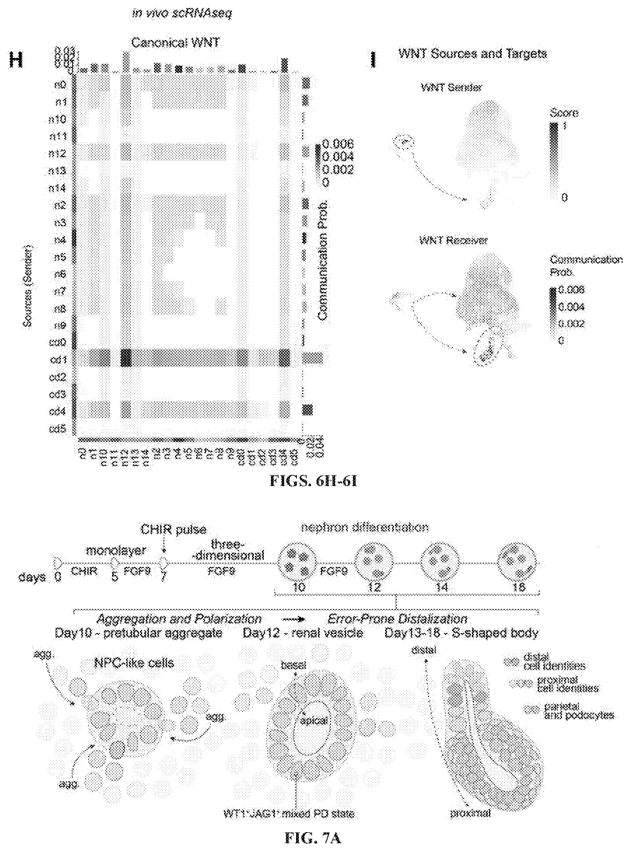

The expression of FZDs, LRPs, and RSPOs were confirmed in scRNA-seq data for NPC WNT-receivers, and similarly reduced expression of RSPO1, 3, 4 was shown in PTA and distal nephron WNT-receivers (FIGS. 6G-6H). Based on these analyses, the study proposes a canonical WNT interaction between cells expressing WNT9B in the CD and CD-adjacent PTA cells positive for WNT4, likely acting as a short-range morphogen driving gene expression in NPCs, PTA and RV.

Overall, these analyses reveal asymmetric transcriptional patterns during nephron development and support the possibility that the WNT11-to-WNT9B transition marks a spatial organizer for the forming nephron, setting the stage for further morphogenesis and differentiation at later stages of nephron development.

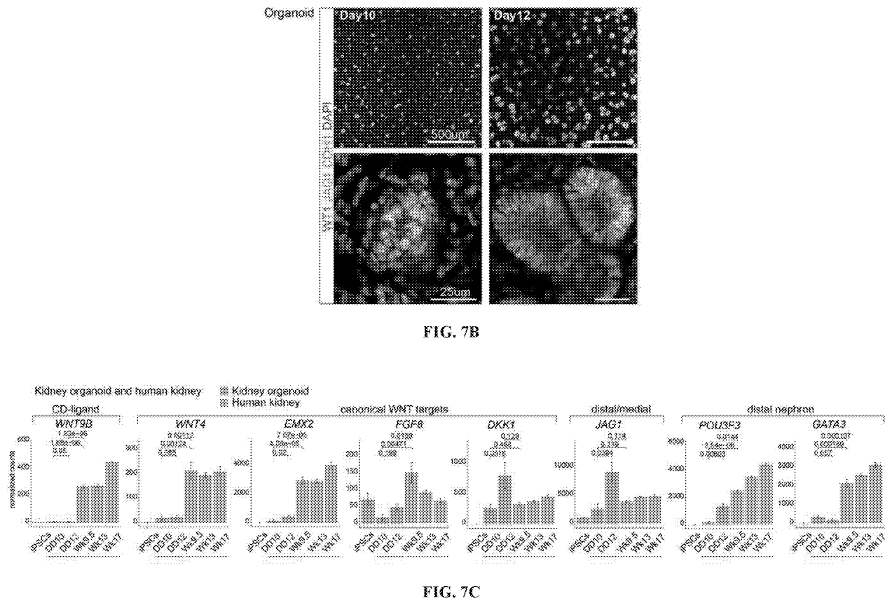

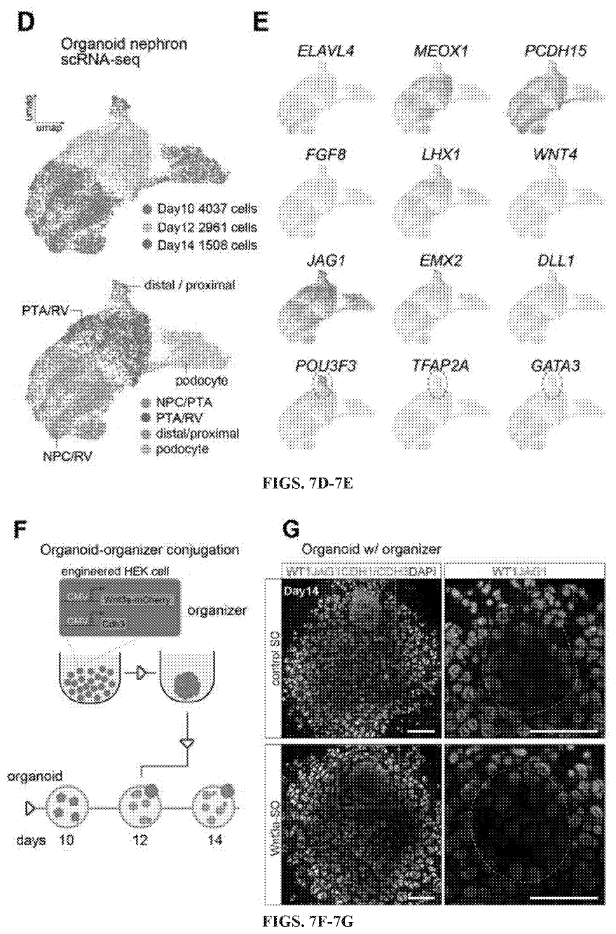

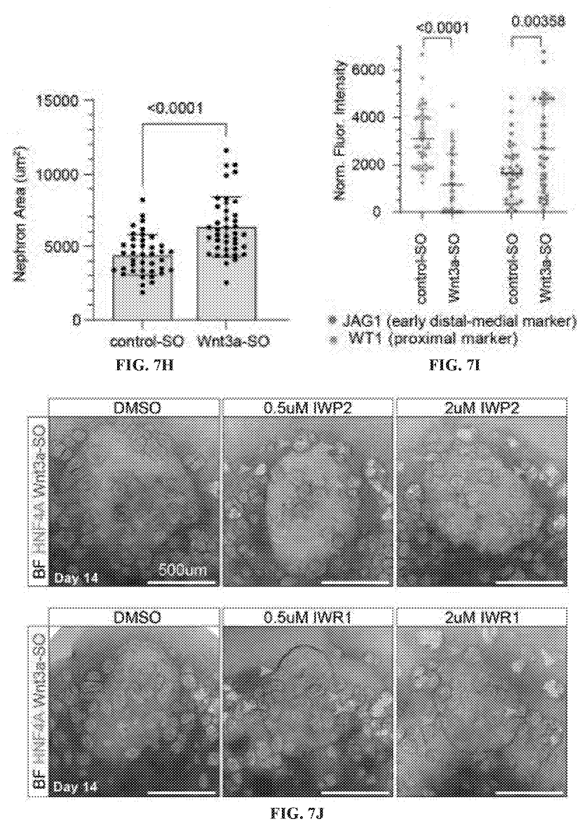

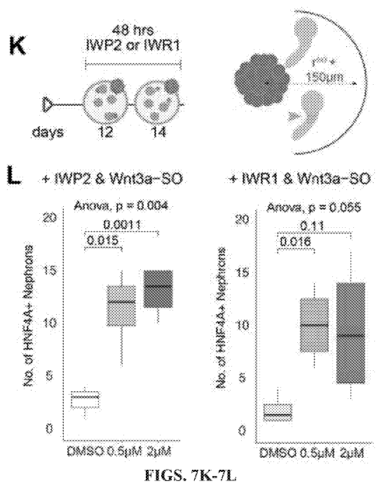

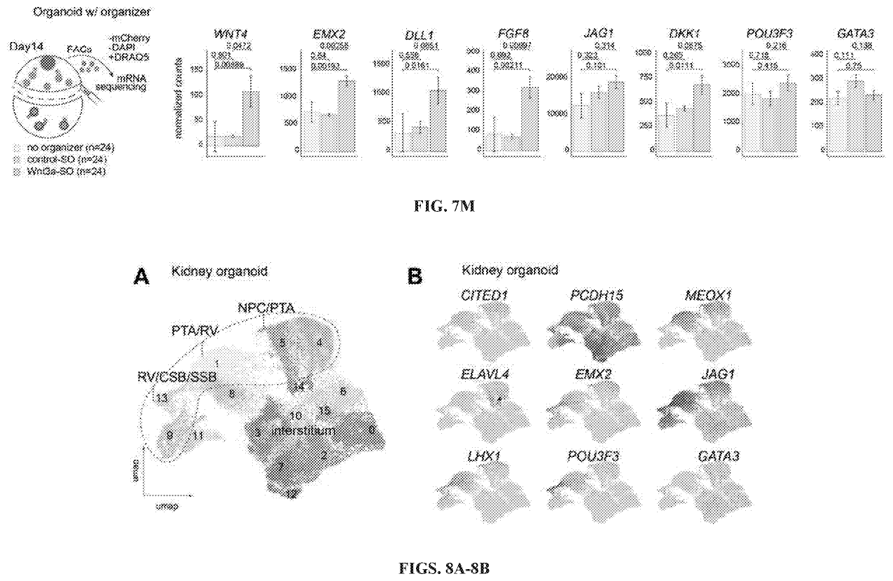

Human kidney organoids lack organized signaling centers and contain non-polarized nephrons: Human kidney organoids and assembloids lack the normal nephron-forming NPC niche where nephrons gradually acquire polarity against an organizing CD. However, they have the potential to be improved by introducing signaling factors that promote self-organizing principles. To define WNT signaling during human kidney organoid formation, the study used a modified Takasato protocol where NPCs are transiently generated, and no CD is present. Briefly in this protocol, 2D hiPSC monolayers were differentiated to metanephric mesenchyme using small molecules and growth factors, and on day 7, transitioned into 3D discs cultured on transwell filters at the air-liquid interface. To drive nephron formation, they are pulsed with Glycogen Synthase Kinase 3 (GSK3) inhibitor CHIR99021 (CHIR) for 1 hour on day 7 to promote canonical WNT signaling and maintained in human recombinant Fibroblast Growth Factor 9 to encourage cell survival until day 12. Thereafter, the organoids were kept in basal medium to enable nephron formation and patterning (FIG. 7A).

Rosettes of cyst-like nephrons of around 35 to 45 μm in diameter, morphologically reminiscent of PTAs, were visible at differentiation day 10 (FIG. 7B). Within 48 hours of forming (day 12), nephrons became fully lumenized epithelia of around 55 to 65 μm diameter, morphologically similar to RVs, as consistent with prior results. These structures expressed epithelial cadherin 1 (CDH1), JAG1, and WT1, markers found at PTA and RV stages of human nephrogenesis. However, their distribution was non-polarized, indicating these structures display radial symmetry, contrary to the CD adjacent-distant asymmetry that is observed in nephrons forming in utero (compare FIG. 7B to FIG. 1E and FIG. 3C).

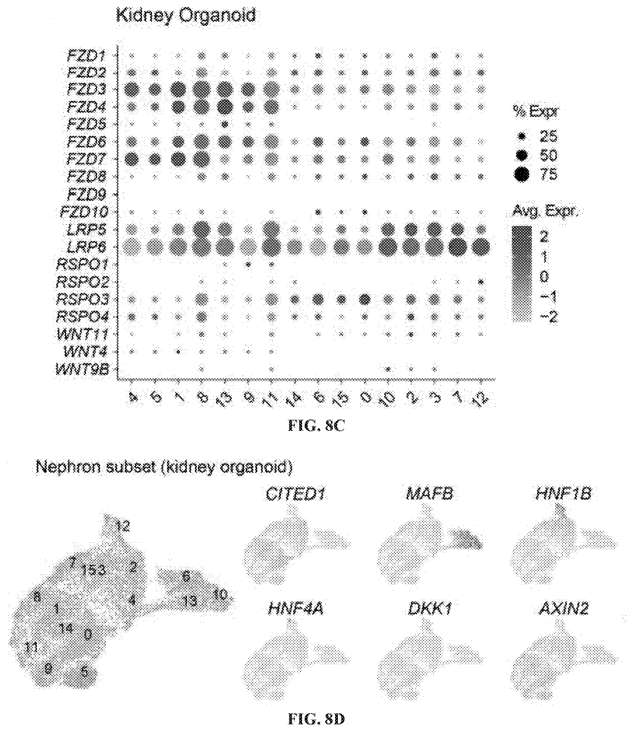

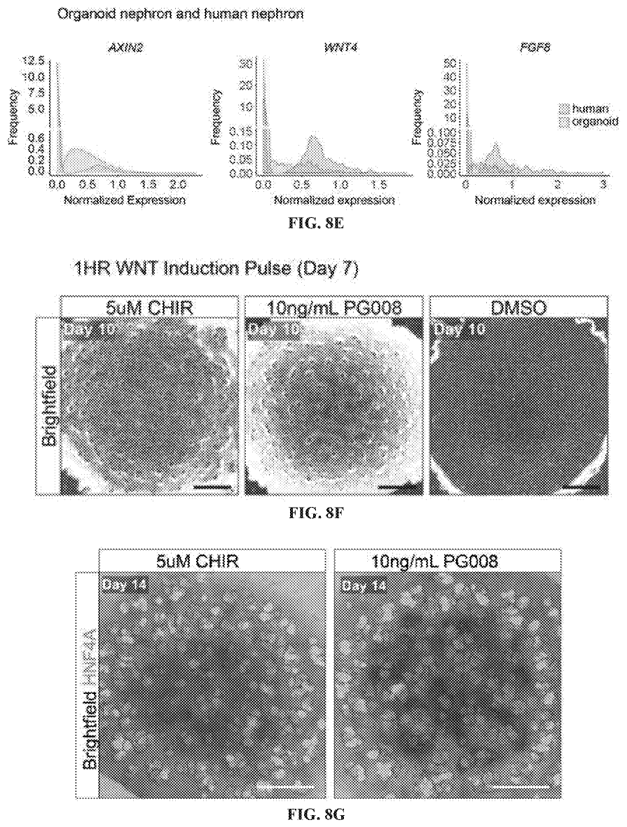

To assess whether organoid nephrons displayed evidence of canonical WNT signaling, the study transcriptionally profiled day 10 to 12 organoids and compared their expression to developing human kidneys rich for nephron formation (weeks 9, 13, and 17). On the ligand side, the CD derived WNT9B ligand transcripts were infrequently detected in organoids while being present in in vivo samples (FIG. 7C). Other canonical WNT ligands that could drive signaling were absent in organoids e.g., WNT3A, while WNT3 was detected at higher counts compared to in vivo. Consistent with a lack of a WNT9B-like driver, WNT targets WNT4, EMX2, and FGF8 were detected at low levels in organoids, while WNT target 51 and secreted WNT antagonist Dickkopf 1 (DKK1) was at comparable levels to in vivo samples. At these early stages of in vitro nephrogenesis, JAG1—a potential WNT target—increased between day 10 and day 12 as did the distal marker POU3F3, but connecting tubule marker GATA3 remained at levels significantly lower compared to in vivo. Neither POU3F3 or GATA3 are thought to be direct targets of TCF/LEF and β-catenin. Examining the organoid nephron transition using scRNA-seq (data from 33), showed that WNT4 and FGF8 mRNA transcripts were absent or low in the organoid nephron and other WNT targets such as AXIN2 and DKK1 were present but infrequently so, particularly when compared to in vivo (FIGS. 7D-7E, FIGS. 8A-8E), confirming the bulk RNA data and highlighting a clear difference in the canonical WNT signal environment between organoid and in vivo development (FIG. 1). Other differences between in vivo and organoids include expression of non-canonical WNT11 in the organoid nephron but not the in vivo nephron (FIG. 8C). On the receptor side, it was observed that organoids did express WNT receptors and signal transduction modifiers from the FZD, LRP, and a subset of the RSPO families (FZD1, FZD2, FZD4, FZD6, FZD7, FZD8, FZD9, FZD10, LRP5, LRP6, RSPO3, RSPO4). Notably RSPO1 was sparsely detected while RSPO2 was undetected. This differs from in vivo observations, where RSPO1 is detected in NPCs (FIGS. 8A-8C, FIG. 4H, FIG. 6G). Thus, organoid nephrons appear primed to respond to canonical WNT ligands such as WNT9B (FZD4, FZD5) or WNT3A (FZD4, FZD5, FZD7), but may not do so because of the absence of the WNT secreting CD. These findings highlight differences in canonical WNT signaling between organoid and in vivo nephrons.

Given that a pulse of WNT signaling is driven at day 7, shortly before the timesteps analyzed here (day 10-12), the study tested if the day 7 CHIR pulse is coupled to nephron aggregation at day 10. To do so, the study removed the CHIR pulse at day 7 and observed no nephron formation (FIG. 8F). Further, the study tested if receptor agonists for WNT receptors could replace the need for CHIR. To do so, the study replaced CHIR with cyclic synthetic peptide PG008 that selectively binds to FZDs and LRP5/6, thus initiating WNT signaling. This resulted in nephrons with similar if not improved aggregation compared to CHIR-pulsed controls, suggesting direct interactions with receptors could be beneficial for nephron derivation in vitro (FIGS. 8F-8H). Next, the study tested if WNT signaling is required after the day 7 WNT pulse. To do so, the study inhibited canonical WNT signaling by adding Tankyrase inhibitor IWR1 or Porcupine inhibitor IWP2 (promoting β-degradation or preventing WNT ligand secretion, respectively) during days 8-10. In the resulting organoids, nephron aggregation and formation was unperturbed by the inhibitors (FIG. 8I), indicating that while a WNT activating event is required at day 7, the aggregation and formation of symmetric nephrons likely follows an independent program in organoids. This is contrary to what is observed in human nephrogenesis in vivo where nephrons are exposed to a sustained WNT signal.

Overall, these data point to a signaling state where organoid nephron aggregation requires a transient canonical WNT signal at day 7 and then proceeds in an environment that is low in WNT signaling.

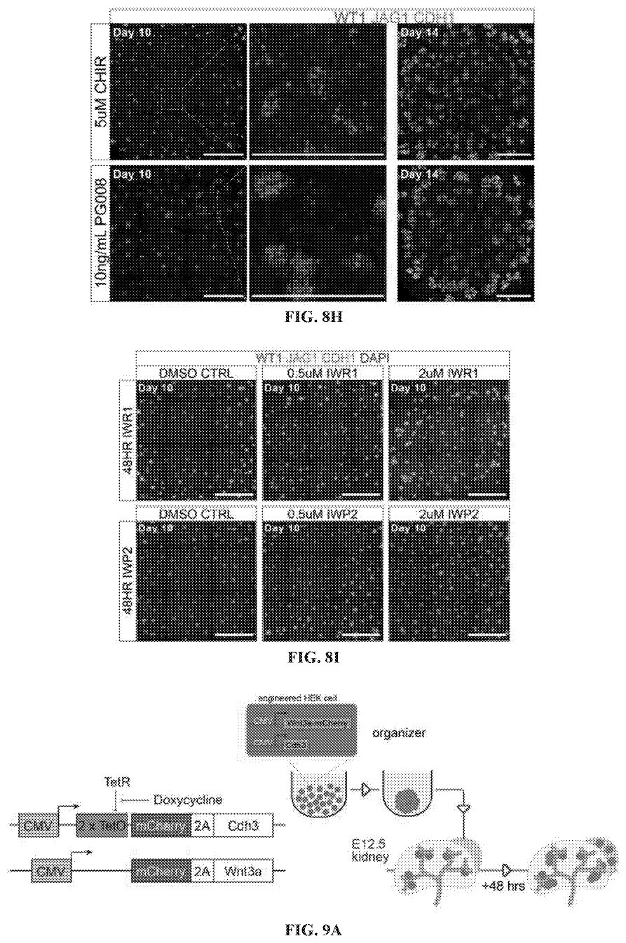

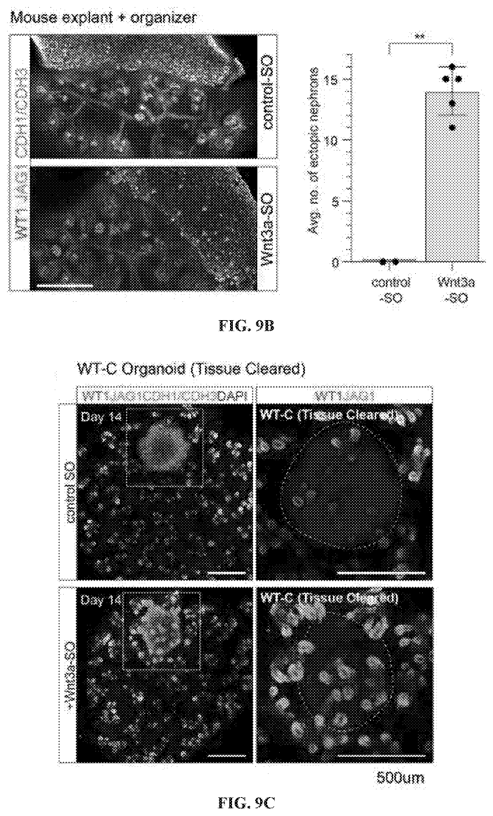

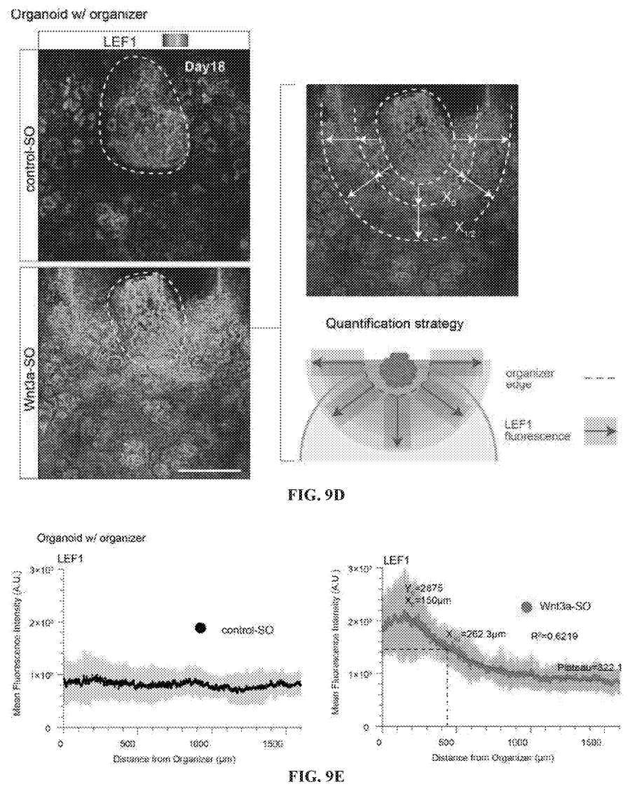

Canonical WNT-secreting signaling centers drive β-catenin target genes in organoid nephrons: Human kidney organoid nephrons have the necessary components to receive canonical WNT signals. To test whether improper expression of WNT target genes could be attributed to a lack of a localized WNT ligand source, the study generated WNT-secreting cell sources. The study followed an approach of building localized signaling centers known as synthetic organizers. Genetically engineered ‘human embryonic kidney’ (HEK) cells, established to produce either a control fluorescent protein (control-synthetic organizer, or control-SO) or Wnt3a (Wnt3a-synthetic organizers, or Wnt3a-SO) in a constitutive manner expressed Cdh3 for self-aggregation and mCherry for visual marking (FIG. 7F and FIG. 9A). Bulk RNA sequencing on Wnt3a-SOs confirmed these only expressed Wnt3a and no other canonical Wnt ligands, while control-SOs did not express any canonical Wnts. The study tested whether Wnt3a synthetic organizers can elicit WNT-dependent nephrogenic and canonical responses by coupling them to embryonic mouse kidney explants, that contain nephron-forming niches. Small (5,000 cells) Wnt3a-SOs and control SOs were assembled in ultra-low adhesion wells for 24 hours and positioned adjacent to embryonic explanted kidneys. Ectopic nephrons (positive for Jag1 and Cdh1) formed in response to Wnt3a-SO but not to SO controls, confirming that Wnt ligands produced by synthetic organizer cells induced a canonical Wnt-response in developing mouse kidney NPCs (FIGS. 9A-9B). To determine if organoid nephrons responded to ectopic Wnt3a ligands, the study introduced Wnt3a-SOs and control SOs on day 12, when nephrons are non-polarized RV-like structures, and allowed them to develop together for 2 days (FIGS. 7G-7I). Nephrons close to control SO were unaffected compared to neighboring structures, but in Wnt3a-SO-conjugated organoids, nephrons close to the Wnt3a-SO grew larger, decreased expression of proximal/podocyte marker WT1 and increased expression of early distal-to-medial marker JAG1 (FIGS. 7H-7I). These results were replicated in hiPSC line WTC11 (FIG. 9C). The effects were therefore consistent with a shift in patterning where the presence of WNT ligands emanating from the synthetic organizers increased distal fates at the expense of proximal ones. The patterning effects were local and affected only nephrons in the vicinity of the organizer and not regions of the organoid farther from the organizer. To explore the spatial range to which β-catenin signaling is affected in the organoid, the study assessed protein levels of β-catenin target Lymphoid Enhancer-Binding Factor 1 (LEF1) and observed increased LEF1 abundance up to hundreds of microns away from the organizer cells (FIGS. 9D-9E). This indicates a capacity of these synthetic organizers to project morphogenetic fields at a distance.

To test that the synthetic organizer effects were due to WNT pathway activation, the study repeated the organizer conjugation experiments and added WNT inhibitors endo-IWR1 and IWP2 between days 12 and 14, to block WNT signaling. The study used an hiPSC line that reports on proximal marker HNF4A (HNF4A^{YFP/YFP}). It becomes activated at day 14 when nephrons are at a SSB equivalent stage and form proximal precursors (FIG. 8I). Wnt3a-SOs blocked HNF4A expression at distances matching the proposed morphogenetic field, but HNF4A-YFP expression was robustly rescued by the WNT inhibitors (FIGS. 9J-9L).

Transcriptional profiling of nephrons close to SOs confirmed their increased expression of WNT targets WNT4, EMX2, FGF8, DLL1 and DKK1 (FIG. 9M). The distal and connecting tubule markers associated with later nephron differentiation POU3F3 and GATA3 were not changed, consistent with a model where early WNT target are activated at early time points after synthetic organizer conjugation. This is also consistent with what was observed in the in vivo timeline, where EMX2 and FGF8 are expressed early in nephrogenesis—prior to POU3F3 and GATA3-adjacent to the WNT11-to-WNT9B transition point (FIG. 1C).

Overall, these results demonstrate that engineered synthetic cellular organizers can express a canonical Wnt ligand that drives β-catenin-mediated responses in human kidney organoids, that these locally program nephrons to influence early PD nephron patterning and favor distal fates, and they introduce developmental programs otherwise absent from hiPSC-derived organoids.

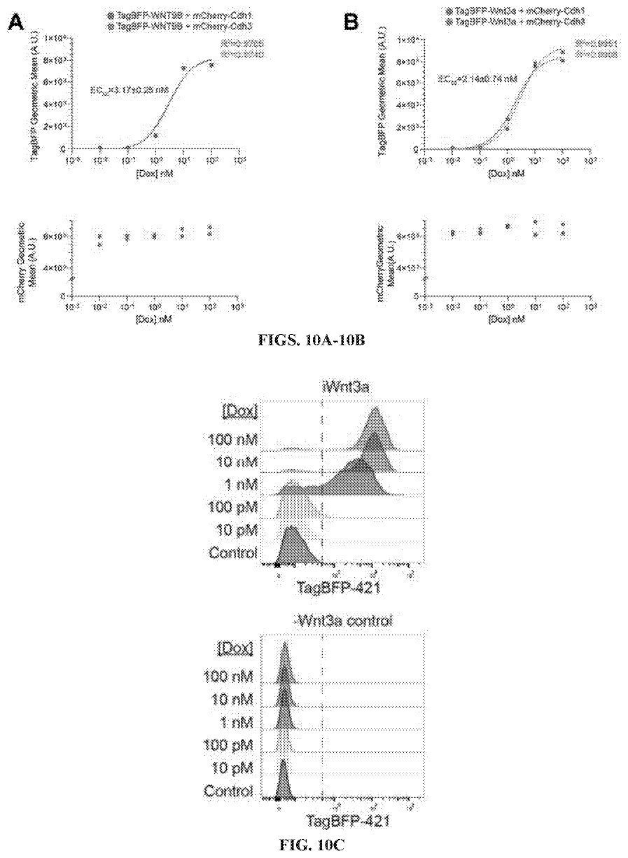

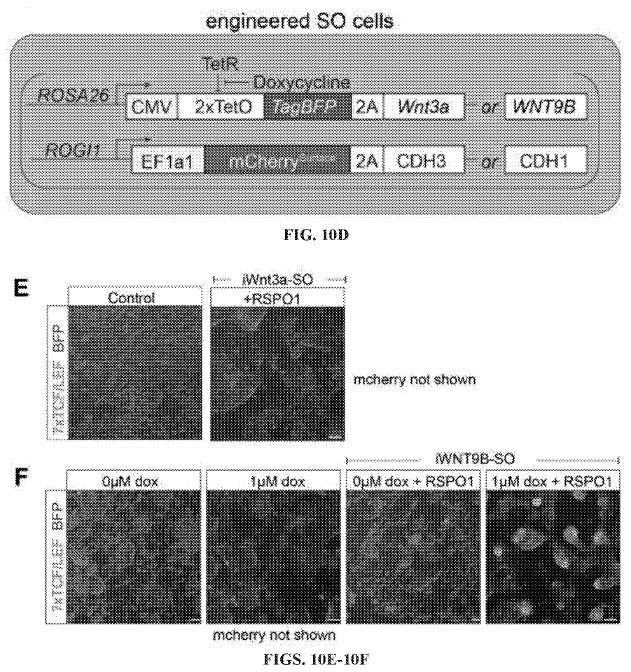

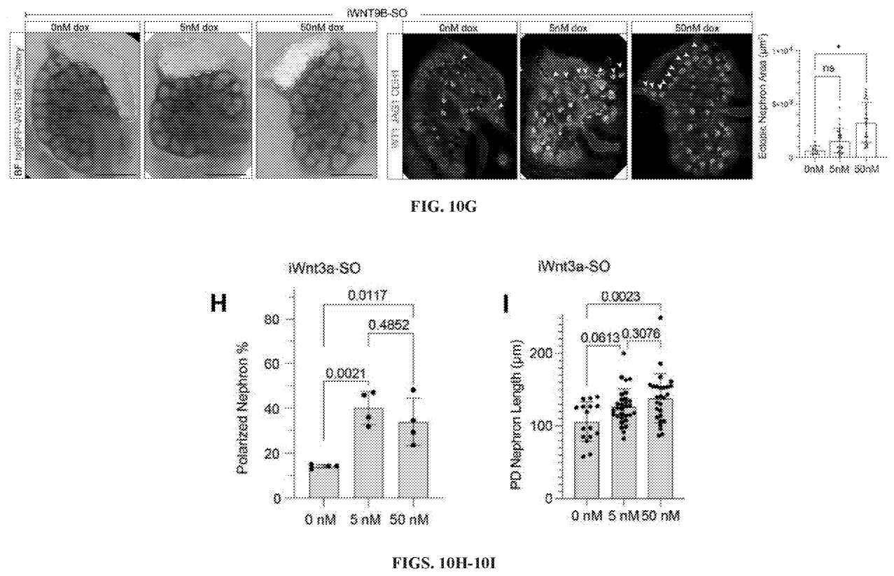

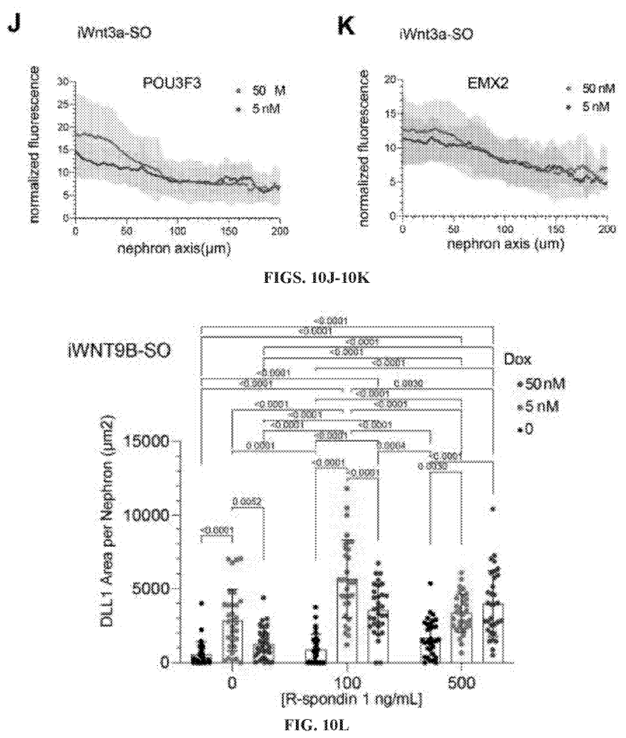

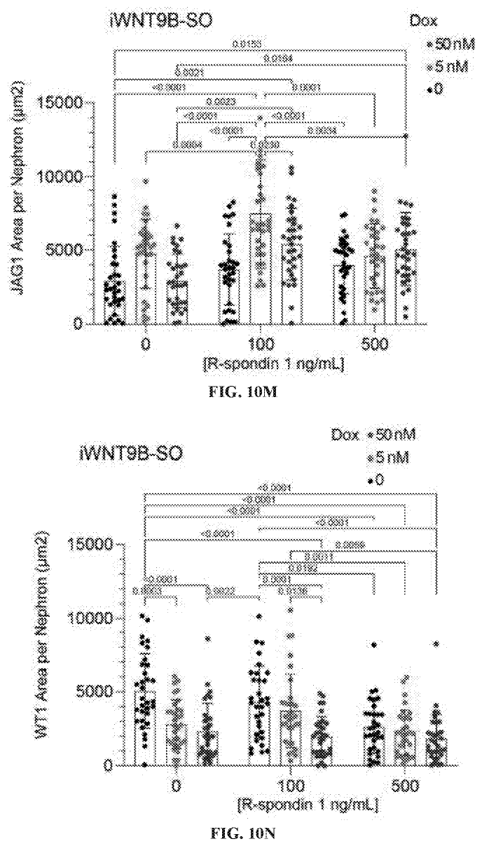

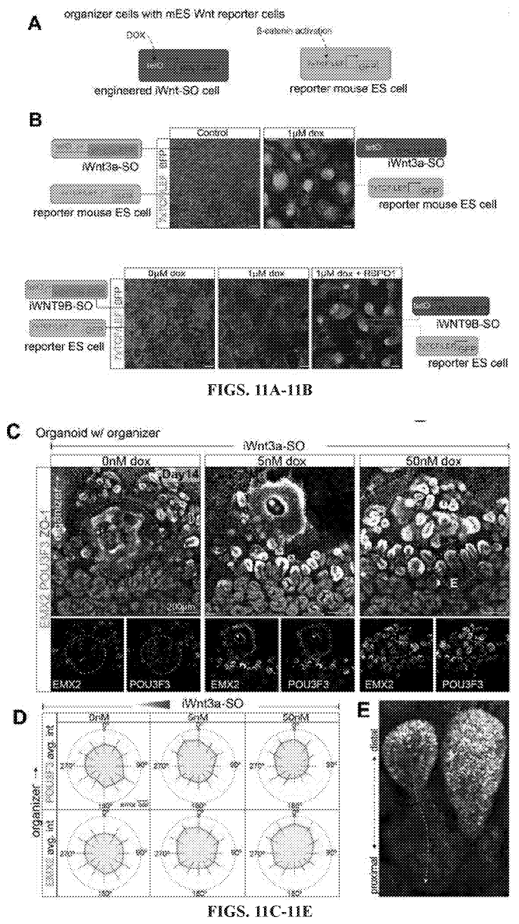

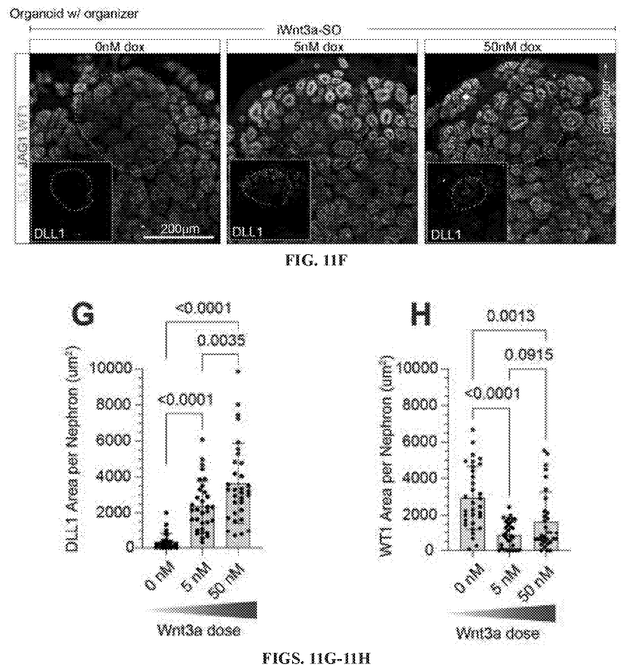

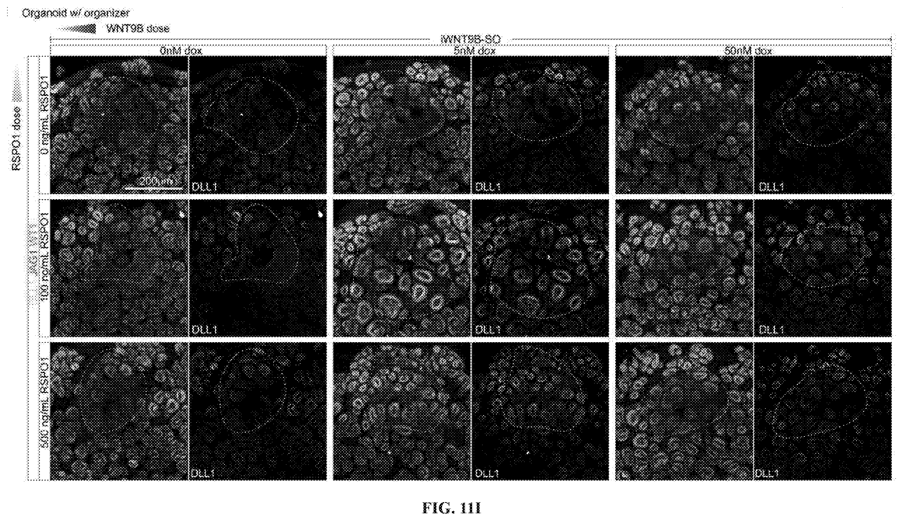

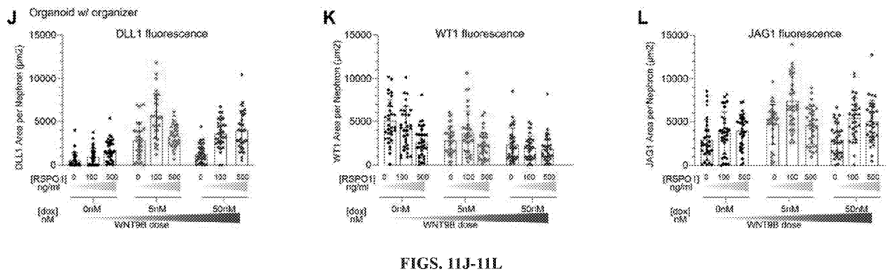

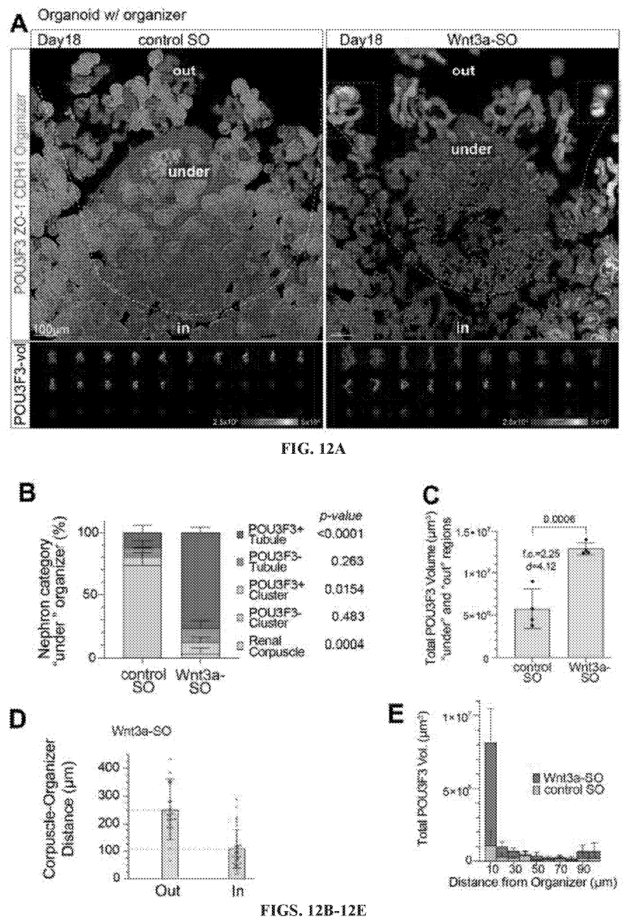

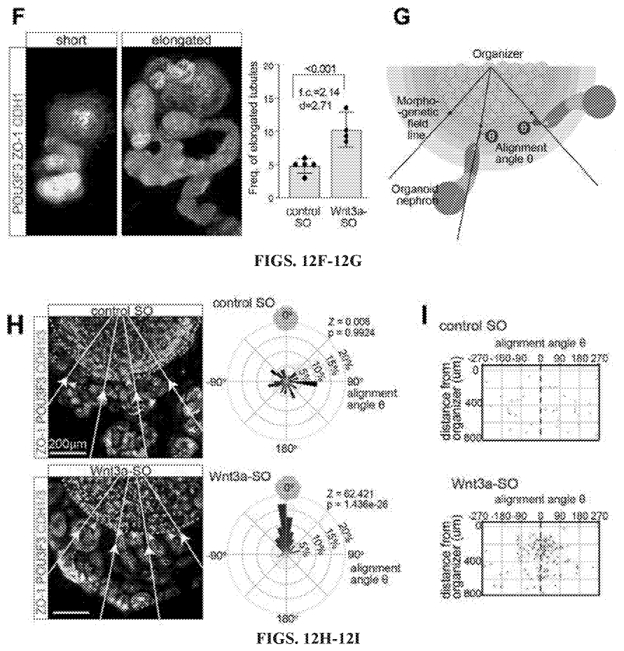

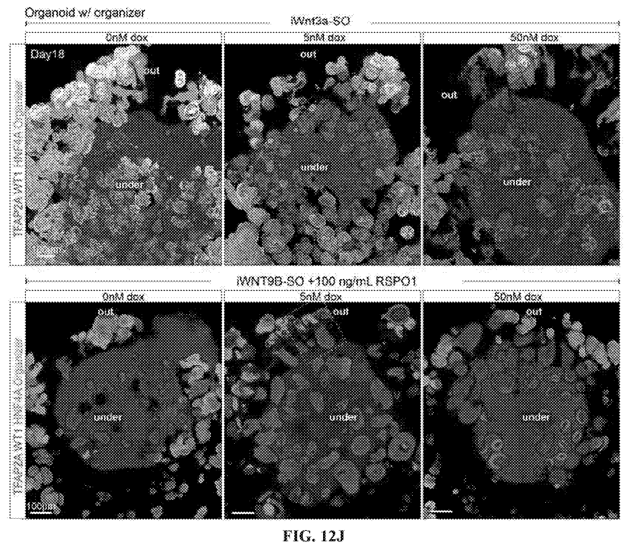

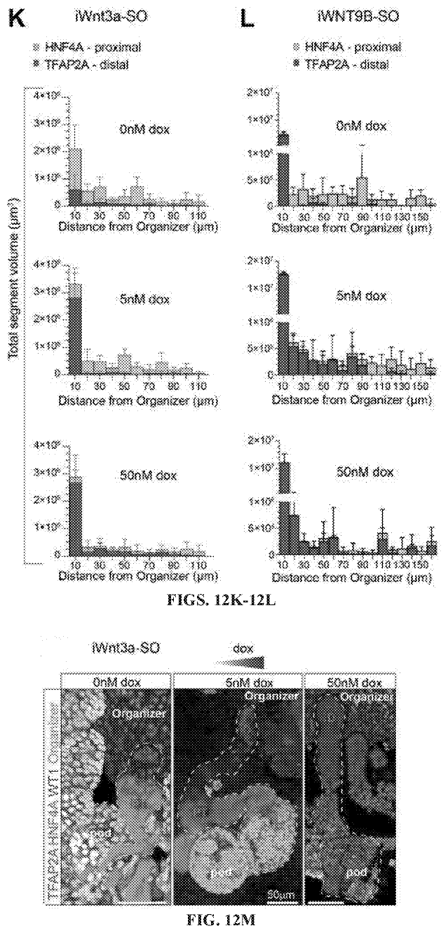

Tunable WNT dosages drive nephron axial symmetry-breaking and morphogenesis: To test if nephron patterning in organoids can be controlled with precision using synthetic organizers, the study engineered and characterized HEK organizer cells with tunable production of canonical Wnt3a and WNT9B ligands. Ligand expression was placed under a dox-inducible promoter, alongside constitutive expression of cadherin protein 3 (CDH3) (FIG. 10D). These inducible WNT synthetic organizers are from here on referred to as iWnt3a-SO and iWNT9B-SO. The study validated the graded expression of the transgenes in these cells via flow cytometry, which showed a sigmoid dose-response curve of inducible expression of TagBFP-2A-Wnt3a transgene from iWnt3a-SO cells (EC₅₀ of 2.14±0.74 nM); and TagBFP-2A-WNT9B from iWNT9B-SO cells (EC₅₀ of 3.17±0.25 nM), in both cases peaking past 10 nM (FIGS. 10A-10C). The study selected 0 nM, 5 nM, and 50 nM doxycycline (dox) to drive WNT expression, and when co-cultured with ES cells carrying a β-catenin TCF/LEF-GFP reporter, the iWnt3a-SO cells induced WNT/β-catenin transcriptional responses in neighboring cells in a dox dependent manner (FIG. 11A, FIG. 10E). iWNT9B-SO cells induced the β-catenin TCF/LEF-GFP reporter when supplemented with RSPO1, a protein promoting WNT receptor abundance at cell membranes and thus potentiating WNT9B-FZD/LRP interactions and downstream β-catenin activity (FIGS. 11A-11B, FIGS. 10E-10F). When conjugated with early mouse kidney explants, iWNT9B-SOs also tunably drove nephrogenesis (Jag1 expressing structures), an in vivo milieu where Rspo1 and Rspo3 are expressed. Small Jag1 expressing structures also formed in 0 nM dox conditions indicating possibly some leakiness of the iWNT9B-SO cells (FIG. 10G).

To regulate early nephron axial symmetry breaking, the study coupled kidney organoids with iWnt-SOs at day 12. After maintaining them for 48 hours in medium with various dox concentrations, the study profiled nephron axial polarities by immunolabelling for distal nephron (POU3F3, EMX2), distal/medial (JAG1: initially distal and then medial), WNT-responsive nephron genes (DLL1 and EMX2—based on FIG. 7M) and proximal (WT1) nephron fates. iWnt3a-SOs polarized and elongating nephron PD axes towards the Wnt3a-SO (FIGS. 10H-10I), increased the abundance of distal markers (FIGS. 11C-11E, FIGS. 10J-10K), and the spatial distribution of the distal markers in RV-like structures was biased to the nephron domains closest to the organizer in a dox dose-dependent manner (FIGS. 11D-11E). DLL1, one of genes in organoid nephrons that responded most strongly to Wnt3a-SOs (FIG. 7M) increased in a dose-dependent manner at a protein level, while WT1 was downregulated (FIGS. 11F-11H). Collectively, these results show that iWnt3a synthetic organizers skew nephron PD patterning and favor distal differentiation at the expense of proximal differentiation.

Since WNT9B is the canonical ligand present in vivo the study tested whether it similarly regulates early nephron axial pattern formation. The study exposed organoid nephrons to 0 nM, 5 nM, and 50 nM dox to drive WNT9B expression and to Ong/mL, 100 ng/mL, and 500 ng/mL RSPO1 and used DLL1, JAG1, and WT1, as in iWnt3a-SO experiments to scrutinize emerging axial patterns. Overall, presence of both RSPO1 and WNT9B resulted in increased abundance of DLL1 and JAG1 and decreased proximal marker WT1 protein (FIGS. 11I-11L). Nephron markers were sensitive to changes in WNT9B and RSPO1. DLL1 and JAG1 were detected in low abundance in organoids in which expression of WNT9B is not induced, peaked at 5 nM dox at any RSPO1 dosage, and was present but lower at 50 nM dox, indicating a complex biphasic response (FIGS. 11J-11K). For both DLL1 and JAG1, RSPO1 dosage also displayed a biphasic trend, where low doses are associated with low abundance, intermediate dose of 100 ng/ml is associated with peak expression, and high dose (500 ng/ml) is associated with lower abundance. Proximal marker WT1 instead followed a monotonic trend of decreased abundance with increased WNT9B dosages (FIG. 11K) (see also FIGS. 10L-10M for statistical analyses). These results show that iWNT9B synthetic organizers locally drive WNT target gene expression and distal nephron programs.

WNT-secreting synthetic organizers tunably orchestrate nephron patterning and morphogenesis: The early nephron axial symmetry-breaking prefigures further morphogenesis and signaling which results in segment precursors being positioned along the nephron PD axis (FIG. 1). To test if exposure to Wnt signaling regulates these processes, as in vivo data suggests, the study conjugated constitutive Wnt3a-expressing synthetic organizers with organoids at day 12 and analyzed patterning outcomes at day 18.

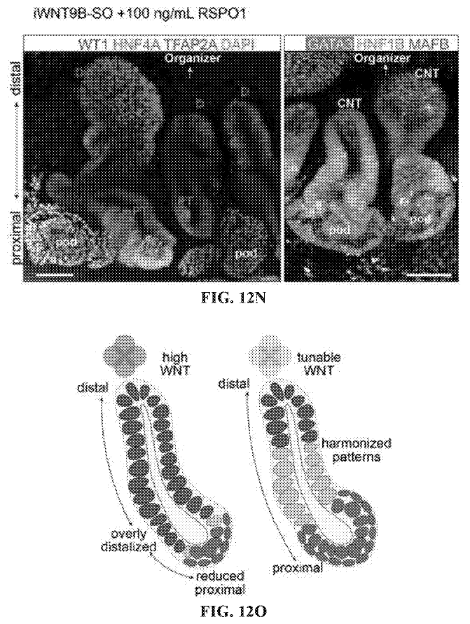

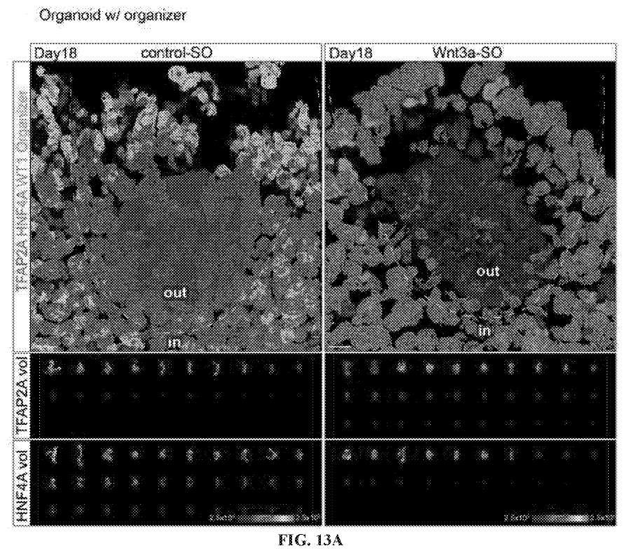

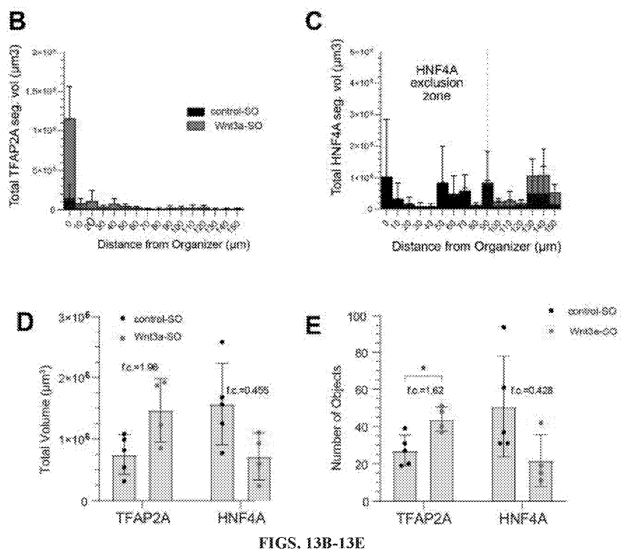

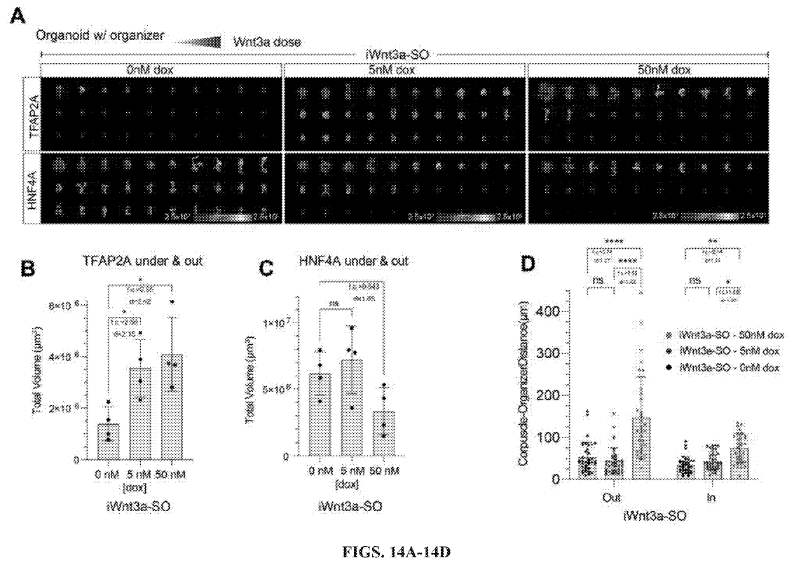

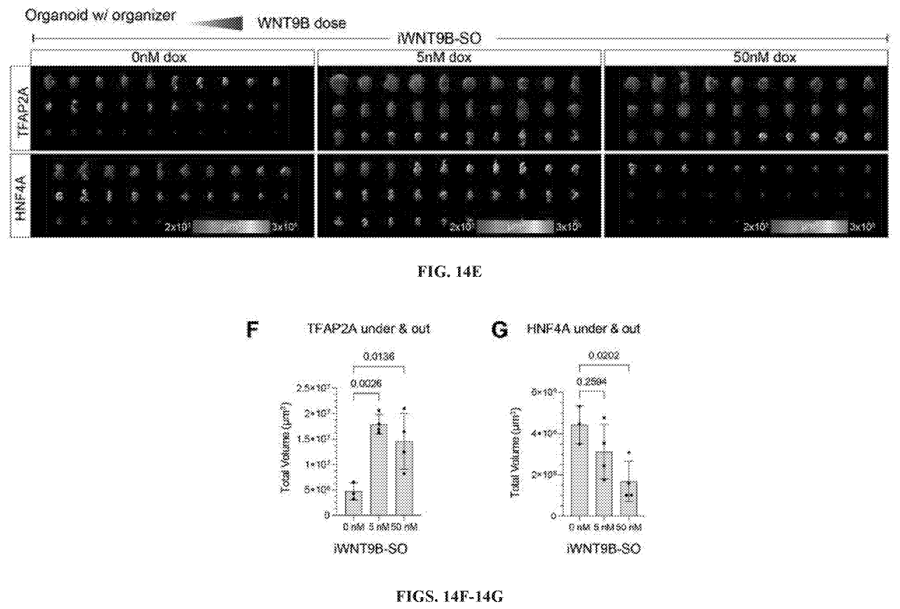

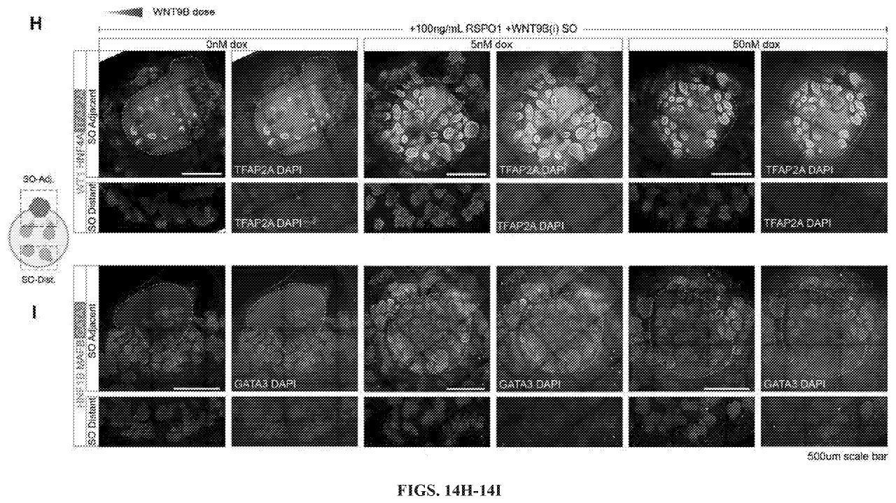

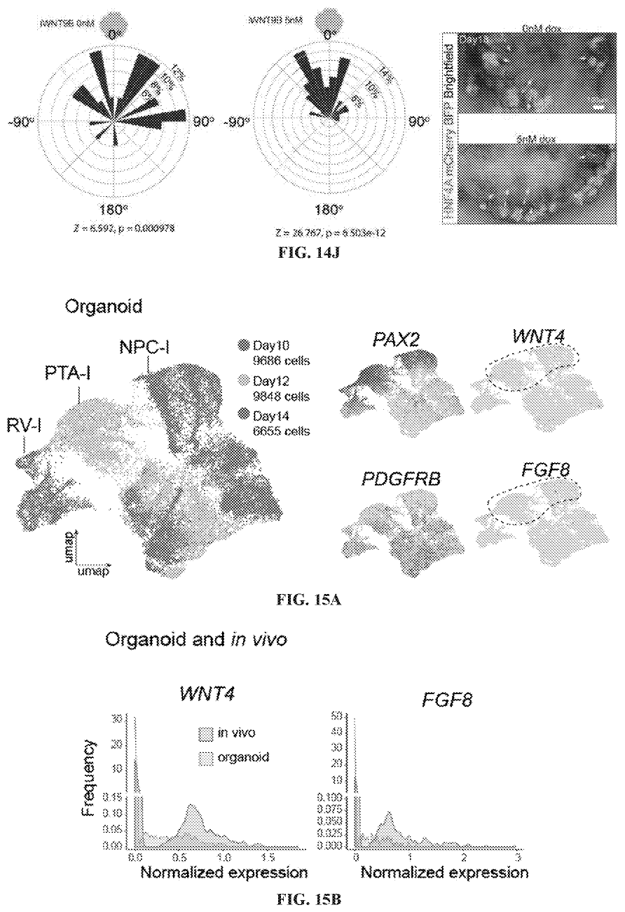

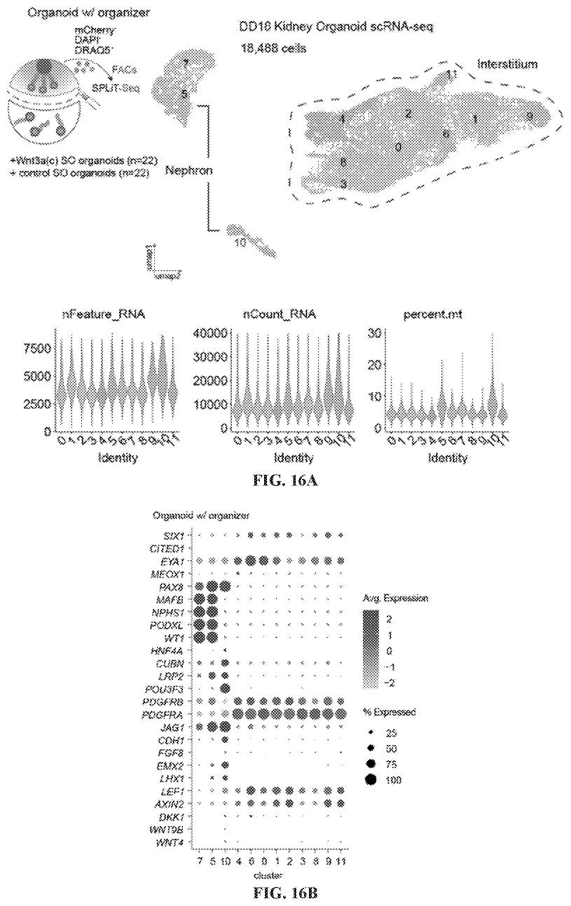

Exposure to Wnt3a-SOs transformed nephrons adjacent to the organizer into distalized tubules (CDH1, POU3F3, TFAP2A) devoid of renal corpuscles-like structures (FIG. 12A and FIG. 13A). Nephrons next to control SOs instead produced renal corpuscles recognizable by nuclear WT1 and strong Zonula Occludens-1 (ZO1) stains and formed small distal domains. To test how nephrons respond to Wnt3a, the study categorized nephrons based on their patterning and morphology—i.e., tubule elongation, upregulation of distal markers, and loss of renal corpuscles and proximal segments. Under control organizers, nephrons primarily included renal corpuscles, whereas under Wnt3a-SOs, differentiation was biased to generate elongated and distalized tubules (FIG. 12B). These distal tubules in nephrons adjacent to Wnt3a-SO grew larger and increased in abundance (FIGS. 12A-12E, FIGS. 13A-13E).

The absence of proximal cell fates (renal corpuscles) close to Wnt3a-SO, together with the distance-dependent increased abundance of LEF1 (FIGS. 9D-9E), indicated that nephrons pattern within a morphogenetic field. Scrutinizing the spatial distribution of renal corpuscles-enriched nephrons showed that these were not detected within a ~100 μm distance of Wnt3a-SOs while in controls they represented the majority of nephrons with a distribution irrespective of distance from the organizer (FIG. 12D). Similar results are observed for HNF4A expressing proximal tubule precursor segments. No HNF4A segments above 2×103 μm3 were found within an 80 μm distance of Wnt3a-SOs, while being abundant in controls (FIGS. 13A-13E). These data demonstrated a patterning effect on nephrons dependent on the distance from the organizers.

Because nephron morphogenesis aligns with the WNT11-to-WNT9B transition point in vivo, and nephrons morphogenetically responded to the tunable iWnt-SOs (FIG. 11, FIGS. 10H-10K), the study scrutinized how nephrons axes and tubules form within the organizer morphogenetic field. Nephrons adjacent to Wnt3a-SO developed a phenotype reminiscent of the elongating distal tubule structures observed in vivo and when these distal tubule-enriched nephrons were categorized as ‘elongated’ or ‘short’ (FIG. 12F, left), the elongated tubules were more common (2.14 fold) in Wnt3a-SO conjugated organoids compared to the controls (FIG. 12F, right).

To assess morphoalignment to the Wnt source, the study mapped the orientation of the nephron PD axes relative to the morphogenetic field lines emanating from the center of the organizer. Nephron PD axes were aligned towards the Wnt-secreting organizers and were stochastic in controls, and this was especially true in organoid nephrons closer to the organizer (FIGS. 12G-12I, FIGS. 13F-12G). This supports a view where Wnt ligands control morphogenesis, growth, and patterning of the developing nephron tubules such that nephrons orient their distal tubules towards the Wnt3a-SO and develop distal nephron cell identities in a distance-dependent manner that simultaneously suppresses proximal tubule and renal corpuscle cell fates.

Since exposure to high amounts of Wnt3a generated non-physiological nephron patterning—expanded distal segments and loss of proximal identities—the study tested if appropriately dosed Wnt3a and WNT9B can accurately pattern nephrons. The study evaluated patterning in day 18 organoids coupled with iWnt3a-SOs and iWNT9B-SOs at different Wnt/dox dosages, by analyzing nephron segment sizes using distal (TFAP2A), proximal (HNF4A) and podocyte/renal corpuscle (WT1) (FIGS. 12J-12L, FIG. 14). 3D reconstruction of confocally scanned nephrons showed unperturbed renal corpuscles and proximal segments underneath iWnt3a-SO in the absence of dox, while in iWNT9B-SO adjacent nephrons instead—in a fixed dose of RSPO1 of 100 ng/mL—there was a slight upregulation of distal markers locally to the SO, indicating a basal leakiness (see also FIG. 14H for contralateral side of organoid showing no distal markers in RSPO1 conditions).

At intermediate Wnt dosages (5 nM), both iWnt3a and iWNT9B-coupled organoids produced nephrons that displayed a continuity of distal-proximal-renal corpuscle segments. These were observed in nephrons adjacent to the organizer and towards the organoid's periphery. In contrast, underneath the organizer, prominent distal TFAP2A-labelled nephron segments formed in the absence of proximal and renal corpuscle segments.

At peak Wnt dosages (50 nM), large structures containing TFAP2A appeared underneath organizers, WT1 expression was increasingly suppressed and tubules containing HNF4A were excluded to positions further from the organizer. Distal segments increased in volume at increasing Wnt doses while correspondingly, HNF4A segment volumes decreased at the highest dose of Wnt (FIGS. 14A-14G). High Wnt doses, mirrored the overt distalization and suppression of proximal identities observed earlier (FIG. 12A).

Closer scrutiny of the intermediate 5 nM dose, revealed individual patterned nephrons with distal (TFAP2A) and connecting tubules (GATA3) being detected alongside proximal tubule precursors (HNF4A and HNF1B) that were connected to renal corpuscle (WT1 and MAFB). These nephrons were aligned with their distal end towards the organizer (either Wnt3a and WNT9B) (FIGS. 12M-12N, FIGS. 14H-14I). This is consistent with distal differentiation being driven near the Wnt source and suggests that intermediate Wnt doses are compatible with a fully patterned nephron. When provided at the right amount, Wnt ligands drive development of well-patterned nephron segments and polarize their axial morphogenesis (FIG. 12O).

### Discussion

A new axial polarity during nephron development: These findings describe an axial polarity detailing how the nephron develops with respect to the CD. This axial polarity is set up when NPCs positioned around the CD tip undergo a gradual mesenchymal-to-epithelial transition to generate nephrons and thereafter the axis persists through the well-characterized developmental morphogenetic steps that prefigure the forming adult nephron morphologies and function. The spatial transcriptional analyses show that a CD adjacent-distant (AD) polarity prefigures the formation of the classic PD polarity. The AD polarity initially traverses the partially epithelialized PTA and can be defined in the PTA by how cells are positioned in relation to the WNT11-to-WNT9B transition point and the WNT9B expressing CD surface (shown in FIG. 1). After the PTA stage, the CD-adjacent domain defines the nephron epithelium that is adjacent to the WNT9B expressing CD stalk. This is evident as the RV and CSB grow, elongate, and folds into the SSB structure. Adding the AD axis as a reference point to describe nephrogenesis assists in defining gene expression patterns and areas of the forming nephron. Conceptually, it integrates with the proposed gradual recruitment model and a time-dependent mechanism dictating nephron fate specification through sequential activation of signaling pathways.

Is the AD axis temporary during nephrogenesis? While the precursor-progeny relationships of AD axial positions need to be defined in future experiments, the data show that molecularly distinct AD regions are prominent in the RV, CSB, and SSB, thus making it hard to envisage how the SSB and subsequent nephron patterning would not be influenced by this polarity. Putative precursors can and are defined by it. For instance, in the SSB the study detected a secondary upregulation of WNT4 in CD-adjacent proximal precursors. However, the dynamics of WNT4 illustrates the asymmetry of the nephron and puts forward an explanation for why podocytes are mosaically labeled with Wnt4^{Cre} fate mapping. Low expression of WNT4 in CD-distant cells is a likely reason for previously observed incomplete Wnt4 Cre-mediated recombination in CD-distant podocytes, while other nephron segments are robustly labeled with Cre-dependent reporters. Moreover, an AD axis also begins to shed light on the chirality of SSBs, as one axis is insufficient, but two axes can define chiral structures.

Insights towards building kidney tissue: Providing organoid nephrons with WNT secreting organizers distalized the nephrons. This demonstrates that human iPSC derived nephrons respond to canonical WNTs by upregulating a transcriptional response akin to that observed in vivo. However, the study found no evidence for an endogenous canonical WNT ligand-receptor pairing in control organoids during the timeframe when nephrons form and pattern. As part of the standard differentiation protocol cells are pulsed with 3-inhibitor CHIR at day 7 and nephrons emerge 3 days later at day 10. Here the study shows that this CHIR pulse can be substituted with synthetic peptides that mimic Wnt3a, which supports the idea that pulsing of canonical WNT signaling is required to form nephrons in organoids. However, the study also demonstrated that inhibition of WNT signaling with Tankyrase and Porcupine inhibitors does not perturb the subsequent aggregation and early differentiation, suggesting the program initiated via the pulsing thereafter acts independent of WNT and β-catenin signaling—unlike what was shown occurs in vivo.

In addition to the transcriptional response to canonical WNT ligands, organoid nephrons aligned their epithelial axes towards the WNT source. Activation of β-catenin in pure NPC cultures in vitro has shown that this is sufficient to drive a cell aggregation program similar to that observed during NPC aggregation into PTAs. NPCs with activated β-catenin signaling become more motile and adhesive to other differentiating NPCs. The data raises the possibility that a canonical WNT acts as one of the attractive signals that coax NPCs to leave their niche and aggregate. In mouse experiments FGF8 has been proposed a chemokinetic factor for NPCs and given that FGF8 and FGF8 are expressed in the distal nephron, it makes it plausible as a downstream target of canonical WNT that facilitates NPC recruitment and aggregation into PTAs. It is however, unlikely to be the sole NPC chemoattractant since NPCs are also recruited from their niche into the proximal ends of forming RVs (where there is no or little WNT9B or FGF8). During this proximal recruitment, highly dynamic NPCs extend long cellular processes from their positions within the NPC niche to those cells within proximal forming nephrons, indicating they respond to a long-range chemotactic signal. Whether these attractive signals are repurposed or act to ensure the axial alignment of the nephron tubule is unclear. The data here shows that Wnt3a and WNT9B can both align and distalize nephrons. A relevant piece of data comes from in vivo experiments when WNT9B is genetically removed from the CD in kidneys with fully formed nephrons. In these experiments, WNT9B knockouts display nephron tubular cysts and loss of normal planar cell polarity and morphology. The work reinforces this notion that nephron responses to canonical WNT ligands are complex and likely not limited to transcriptional regulation. This single ligand canonical WNT9B/WNT9B ligand might therefore act in several capacities with different cellular and anatomical constraints. The data suggests that in vitro synthetic Wnt3a and WNT9B organizers project morphogenetic fields across long distances, perhaps beyond that which has been previously described where the ligand reaches a few cell layers. The mechanism underlying this will require further study, but it provides a pragmatic approach to controlling nephron patterning in vitro.

In summary, this study proposes that nephrons possess a distinct axial polarity, show its relationship to a cell organizer in vivo, and demonstrates that these patterning and morphogenetic events can be reconstituted in stem-cell-derived nephrons via synthetic cell organizers. From the perspective of engineering cell-derived kidney tissue, the duality of one signal driving two processes is advantageous as it can be used to drive distal nephrons towards defined positions in a common epithelial drainage system. It is expected that these findings and new approaches to provide a framework for understanding human nephron development and to provide strategies for engineering a kidney tissue capable of draining urine to a common epithelium.

### Materials and Methods

Animal studies: Institutional Animal Care and Use Committees (IACUC) at the University of Southern California reviewed and approved all animal work as performed in this study (protocol #21179). All work adhered to institutional guidelines. Timed matings were set up to recover embryos at the appropriate age (embryonic day 12.5), sex not known. C57/B16J were used. Kidney explants were cultured as previously described.

Kidney samples: Kidney samples were collected under Institutional Review Board approved protocols (USC-HS-13-0399 and CHLA-14-2211) as for previous study. Following patient decision for termination, informed consent for donation of the products of conception for research purposes was obtained and samples collected without patient identifiers. The person obtaining informed consent was different than the physician performing the termination procedure, and the decision to donate tissue did not impact the method of termination. Developmental age was determined according to the American College of Obstetrics and Gynecology guidelines. The kidney samples were 13 and 16 weeks of gestation with no sex reported.

Cell culture: Human iPSCs were grown on matrigel-coated (Corning: 354277) 6-well plates in E8 medium (Gibco: A1517001). Kidney organoids were derived using a previously published differentiation protocol. HEK-293 cells were cultured in Dulbecco's Modified Eagle Medium (DMEM; Gibco: 11965092) supplemented with 10% fetal bovine serum (FBS; Genesee Scientific: 25-514, lot P085877), 2 mM L-glutamine (Gibco: A2916801), 100U/ml; 100 μg/ml penicillin/streptomycin (Gibco: 15140122) and 0.1 mM β-mercaptoethanol (Sigma Aldrich: M3148-25ML). HEK-293 cultures were grown to 90% confluence and passaged every 3-4 days at a 1:10-1:12 ratio. HEK-293 cells were harvested by aspirating culture medium, washing with phosphate-buffered saline (PBS; Gibco: 10010049), incubating with 0.35% trypsin (Genesee Scientific: 25-510) at 37° C. for 3-5 minutes, neutralizing with double the volume of fresh medium, and collecting the desired ratio for passaging and organizer production.

Directed differentiation to generate kidney organoids: Kidney organoid differentiation was adapted from published protocols and optimized for nephron lineages. Biological replicates were generated from different iPSC batches. At 70-80% confluency iPSCs were rinsed with 1×PBS (Thermo Fisher Scientific, 10010049), isolated using TrypLE Select Enzyme (Thermo Fisher Scientific, 12563011) for 6 minutes at 37° C., enzymatic reaction neutralized in Essential 8 media, and cells collected and resuspended in Essential 8 media with 10 μM Y-27632. 17,500 cells were plated per well into a 12-well plate well onto 5% biolaminin 521 LN and differentiation started 6 hrs post plating in TeSR-E6 (Stem Cell Technologies, 05946) with CHIR99021 (Tocris, 4423). Thereafter, media was supplemented with CHIR99021 for 5 days. CHIR99021 concentrations were optimized for each cell line. At day 5, media was changed for TeSR-E6 with 200 ng/mL FGF-9 (R&D Systems, 273-F9) and 1 μg/mL Heparin (Millipore Sigma, H4784). At day 7, cells were isolated using TrypLE and resuspended in TeSR-E6 with 10 μM Y-27632. 200,000 cells were seeded into round-bottom non-adhesive 96-well plate wells and allowed to self-aggregate. 3D organoid aggregates were manually transferred to transwell filters (Corning, 3450; Stem Cell Technologies, 100-1026), pulsed with TeSR-E6 with CHIR99021 for 1 hour and thereafter in TeSR-E6 with 200 ng/mL FGF-9 and 1 μg/mL Heparin until day 12. From day 12 onward, organoids were cultured in TeSR-E6 alone.

DNA constructs: For ROSA26/ROGI1 locus targeting, gRNA-encoding oligos were cloned into pSpCas9-GFP as described previously. Plasmids were screened via Sanger sequencing using the LKO.1 5′ primer. For insertion of TagBFP-2A-Wnt3a/WNT9B into ROSA26, TagBFP was PCR-generated from an in-house construct (pTH11); 2A was added downstream through two PCR rounds using sequential reverse primers. Wnt3a cDNA was PCR-generated from +Wnt3a-SO gDNA. WNT9B isoform-201 cDNA was acquired as a synthetic gene fragment following human codon GC-content optimization (Twist Biosciences). TagBFP/WNT fragments were assembled into KpnI-linearized pHDR-ROSA26-MCS vector established previously. For insertion of mCherry^{Surface}-2A-Cdh1/Cdh3 into ROGI1, the EF1α promoter was PCR-generated from pHDR-ROSA26-MCS, mCherry^{Surface}-2A from an in-house construct (pMMP250-Cdx2), and Cdh1/Cdh3-pA from E-cell/P-cell gDNA. EF1α/mCherry^{Surface}/Cdh fragments were assembled into BamHI-linearized pHDR-ROGI1-MCS vector established in-house based on published reports. All PCRs were performed using Q5 polymerase master mix (NEB: M0492S); primer sequences are in the key resources table. Assemblies were performed using 30 fmol vector, 50 fmol per insert, HiFi DNA assembly reaction mix (NEB: E2621L), in 20 μl final volumes at 50° C. for an hour. Volumes corresponding to 2 fmol of vector were used to transform Stellar competent bacteria via 42° C. heat-shock for 30-35 seconds; 9 parts SOC medium were added to 1-part bacteria, transformants were outgrown at 37° C., 250 rpm for 40 minutes, and one-tenth of the outgrowth was plated on Luria-Bertani agar plates containing 100 μg/ml ampicillin. Four-to-six colonies were expanded in 3-4 ml ampicillin-supplemented broth and purified plasmids were screened via restriction digestions and full plasmid sequencing (Primordium labs).

Cell engineering: One-microgram pSpCas9-gRNA and 3 μg pHDR-ROSA26/ROGI1 constructs were added into 125 μl OptiMEM together with 5 μl P3000 reagent. The mixture was added into a pre-vortexed solution containing 125 μl OptiMEM and 5 μl Lipofectamine3000 reagent (ThermoFisher: L3000001). Solutions were incubated on-site for 15 minutes. HEK-293 6-well cultures at 70-80% confluence were gently washed in PBS, replenished with 1 ml OptiMEM, and transfection solutions were decanted dropwise while rocking the plate. Transfectants were incubated at 37° C., 5% CO₂ for 6 hours, and replenished with culture medium. Two days later, cultures were refed with medium containing 10 μg/ml puromycin (pHDR-ROSA26-WNT) or 800 μg/ml G418 (pHDR-ROGI1-Cdh). Drug-supplemented medium was replenished every 2-3 days for 12-14 days total, whereupon 1 μM doxycycline was added for 24 hours. Cells were sorted on the basis of a double positive (mCherry and TagBFP) profile, cultured for 5 days without doxycycline, and re-sorted on the basis of mCherry positive and TagBFP negative profile.

Evaluation of organizer doxycycline tuneability: One-hundred-thousand cells were seeded in 12-wells. The next day, cultures were refed with fresh medium supplemented with 10-fold serial dilutions of doxycycline. Twenty-two hours after supplementation, cultures were harvested, resuspended in 0.6 ml PBS-2% FBS, filtered through flow cytometry tube cap filters and analyzed on a BD FACSAria II cytometer.

Synthetic organizer production: On organoid differentiation day 11, HEK cultures were harvested and cell concentrations determined using a Countess II FL automated cell counter (Invitrogen). Five-thousand cells per spheroid times desired number of spheroids were pelleted and resuspended in doxycycline-supplemented medium (50 μl per 5×10³ cells). Fifty microliters were decanted in Nunclon Sphera 96U-well round bottom plate wells (Thermo Scientific 174929), the plate was centrifuged at 100 g for 2 minutes, and placed in a 37° C., 5% CO₂ incubator overnight.

Surgical placement of synthetic organizers onto organoids: On organoid differentiation day 12, organizers were pooled and neutralized into a petri dish (Midland Scientific, P7453) pre-filled with 5 mL TeSR-E6 (Stem Cell Technologies, 05946) in the corresponding doxycycline concentration used during organizer generation (see section Synthetic organizer production). Under a dissecting microscope, synthetic organizers were pipetted onto the organoid and adjusted using syringe needles with care to avoid tears and punctures. Residual media was aspirated using a pipette.

Molecular inhibition of WNT pathway in organoids without and with organizers: Organoids without organizers: on organoid differentiation day 8, kidney organoids were treated for 48 hours with TeSR-E6 (Stem Cell Technologies, 05946) with 200 ng/mL FGF-9 (R&D Systems, 273-F9) and 1 μg/mL Heparin (Millipore Sigma, H4784) and titrated concentrations of endo-IWR1 (0.5 μM, 2 μM, DMSO control) (Tocris, 3532) or IWP2 (0.5 μM, 2 μM, DMSO control) (Tocris, 3533). After 48 hours on differentiation day 10, organoids were fixed for immunostaining and tissue clearing (see section Immunostaining and clearing). Organoids with organizers: On organoid differentiation day 11, synthetic organizers expressing constitutive Wnt3a-mCherry and doxycycline inducible Cdh3-mCherry were produced as described previously (see section Synthetic organizer production) and with 2 μM IWP2. On day 12 during organizer placement (see Surgical placement of synthetic organizers onto organoids), organoids with normal WNT-secreting organizers were cultured for 48 hours with TeSR-E6 with endo-IWR1 (0.5 μM, 2 μM, DMSO control) and organoids with IWP2-treated organizers were cultured for 48 hours in TeSR-E6 with IWP2 (0.5 μM, 2 μM, DMSO control). After 48 hours, organizer-coupled organoids were imaged.

Kidney organoid nephron induction via Wnt3a alternative peptide: On differentiation day 7, organoid aggregates are pulsed for 1 hour with 5 uM CHIR99021 (Tocris, 4423), 10 ng/mL Wnt3a alternative peptide (Peptigrowth, PG-008), or DMSO control (Sigma-Aldrich, D5879). After the pulse, organoids are cultured as previously described (see Directed differentiation to generate kidney organoids).

mRNA-sequencing and data analyses: Samples were prepared and purified according to the RNeasy Mini Kit (Qiagen, 74104). For iPSCs, one well from a 6-well plate was collected prior to kidney organoid differentiation. Whole organoid samples were collected for differentiation days 10 and 12 (2 organoids per biological replicate). Organoid regions adjacent to synthetic organizers were surgically isolated using a sichel knife (Fine Science Tools, 10073-14), and subsequently dissociated and purified sorted for DRAQ5+, DAPI−, and mCherry−. Wnt3a-SOs and control-SOs were cultured as 5,000 cell aggregates in a 96-well U-bottom plate for 24 hours before collection. Purified RNA was sent to Novogene for paired end sequencing. Reads were aligned to the hg38 Ensembl 105 annotation using star version 2.7.10a. A noise-reduction filter was applied to keep genes in which the maximum count in or at least one sample was greater than or equal to 10. Partek Flow version 10.0 was then used to normalize raw feature counts for Wnt3a-SOs and control-SOs following the transcripts per million (TPM) normalization method. Separately, iPSC, organoid, and previously published human kidney datasets were normalized using the DESeq2 median ratio method 13. One-sided Welch's tests were used to perform statistical analyses using t.test( ) with the parameter var=F in R. Data visualization was made using ggplot2( ) in R.

Quantification of LEF1 abundance in morphogenetic fields generated in organizer-coupled kidney organoids: Kidney organoids were coupled with WNT organizers or control organizers on day 12 and collected on day 18 for immunostaining (see section Immunostaining and clearing). Samples were imaged via widefield fluorescence microscopy and imaging data was processed on ImageJ/FIJI by measuring LEF1 abundance along kidney organoids as previously done for nephron axes 1. Briefly, five lines were drawn along the organizer's edge to make a 180° arc facing the organoid using the straight-line tool. To capture all fluorescence, line widths were set to 500 and care was used to ensure line widths did not overlap. Intensity along each line was averaged per organoid, and a best-fit curve over the distance of decreasing LEF1 fluorescent intensity was generated using a one phase decay model.

Quantification of protein abundance in organoid nephrons: Protein abundance was measured based on fluorescent intensity along the nephron axis as previously described, starting at the distal end adjacent towards the organizer. POU3F3 and EMX2 were quantified separately.

Quantification of polarized POU3F3/EMX2 protein distribution: Nephrons within a 3-nephron-length distance from organizers were screened for POU3F3/EMX2 protein asymmetry: in line with the WNT morphogen field described in FIGS. 9D-9E. For radar plots, coordinates at the center of each nephron's lumen, and at the point around the nephron's perimeter closest to the organizer, were recorded with the point tool. 10×10 μm circles were drawn with the oval selection tool and placed on DAPI-marked nuclei along the nephron's perimeter; coordinates and mean grayscale intensities for all channels were quantified within each circle. POU3F3 and EMX2 intensities were normalized to DAPI, expressed in coordinates relative to the organizer and nephron lumen using a custom Python script, and averaged for each experimental group. For axial line plots, a straight line was drawn from the nephron's point closest to the organizer, through the lumen's center, and to the opposite end to bisect the nephron. For each half, 20-width curved lines were drawn along DAPI-marked nuclei from the near-organizer point to the opposite end using the freehand line tool, and channel grayscale intensities along the line's length were measured. POU3F3 and EMX2 intensities along the line's length were normalized to DAPI and averaged for the two halves of each nephron; results were averaged across nephrons of each experimental group.

Kidney Organoid Single-cell RNA sequencing and analyses: Day 10, 12, and 14 kidney single cell RNA sequencing was performed as described previously. Developing kidney single cell profiles (week 14 and 17 post conception) were obtained from published datasets and integrated to generate the in vivo nephrogenesis framework.

Spatial transcriptomic analyses of developing kidneys: Whole kidneys were fixed at 4° C. for 4 hours in 4% PFA (Electron Microscopy Sciences, 15710), thoroughly washed with PBS, and embedded in OCT. 10 um sections were prepared for Xenium and GenePS platforms as per manufacturers' recommendations (10× Genomics, Spatial Genomics). Gene probe sets (249-250 genes) were designed to capture all kidney populations at week 16 and the nephrogenic gene expression program. Nephrogenic niches and stages were identified based on morphologies and gene expression patterns. Transcript positions were quantified using R-studio and Fiji to examine transcript coordinates relative to the collecting duct tip and stalk using WNT11 and WNT9B as markers. Genes presented in FIG. 1, FIG. 2, and FIG. 3 are as follows: Wnt Family Member 11 (WNT11), Wnt Family Member 4 (WNT4), Wnt Family Member 9B (WNT9B), SIX Homeobox 1 (SIX1), Lymphoid Enhancer Binding Factor 1 (LEF1), Platelet Derived Growth Factor Receptor Beta (PDGFRB), Platelet And Endothelial Cell Adhesion Molecule 1 (PECAM1), WT1 Transcription Factor (WT1), MAF BZIP Transcription Factor B (MAFB), LIM Homeobox 1 (LHX1), Jagged Canonical Notch Ligand 1 (JAG1), Empty Spiracles Homeobox 2 (EMX2), GATA Binding Protein 3 (GATA3), HNF1 Homeobox B (HNF1B), Fibroblast Growth Factor 8 (POU3F3), POU Class 3 Homeobox 3 (TFAP2A), Transcription Factor AP-2 Alpha (IRX1), Iroquois Homeobox 1 (PAPPA2), Hepatocyte Nuclear Factor 4 Alpha (HNF4A), Hepatocyte Nuclear Factor 4 Gamma (HNF4G), Protein Tyrosine Phosphatase Receptor Type O (PTPRO), Spalt Like Transcription Factor 3 (SALL3).

Gene list design for spatial transcriptomic probe sets: To design the probeset for the spatial transcriptomic analyses (Xenium and seqFISH) week 17 and 18 human kidney snRNA-seq datasets (N=4; 2 females, 2 male) were used to determine differentially expressed genes expressed in different cell types. Briefly, datasets were generated using standard Seurat workflow and quality control, and differentially expressed genes were identified using FindAllMarkers( ). The top 5 genes per cluster were visualized using FeaturePlot( ) to ensure differential expression. Gene selection was biased to include more genes in nephron lineage. Finally, putative lineage marker genes in the developing kidney were manually added. Gene lists were finalized per Spatial Genomics (seqFISH) and 10× Genomics (Xenium) recommendations after independent quality control using the snRNA-seq dataset described and weeks 13-21 human kidney bulkRNA-seq datasets.

Human kidney single-nuclear RNA and spatial transcriptomic integration and label transfer: Previously published Single-nuclear RNA sequencing datasets (Kim, S. et al. Comparative single-cell analyses identify shared and divergent features of human and mouse kidney development. Dev Cell (2024)) were subset based on gene expression of known marker genes in the developing human kidney's nephron precursors to the S-shaped body stage nephron using Seurat. Subset cells were subsequently integrated with two spatial transcriptomic (seqFISH) datasets using R, which generated the integrated principal component space and UMAP projection. UMAP projections were used for visualization, and snRNA-seq Seurat clusters were transferred onto seqFISH data by querying nearest neighbors (KNN=30) of each spatial cell in the integrated principle component space. Other metadata was transferred using Seurat clusters as an anchor. Integrated UMAPs were generated with ggplot2, and Spatial FeaturePlots were generated using Seurat.

RNA scope and quantification of WNT4 expression: Whole kidneys fixed and prepared as for spatial transcriptional analyses were processed for RNA scope as previously described 15. RNAscope images of 8 renal vesicle-staged nephrons stained for WNT4, WNT11, WNT9B, KRT8 and DAPI were processed on ImageJ/FiJi. The nephron's lumen center and WNT11/WNT9B boundary were marked with the point tool. WNT11, WNT9B and WNT4 puncta were registered using the multi-point tool. Lines were drawn between the nephron's lumen center point and the WNT11/WNT9B boundary using the straight-line tool, and between the nephron's lumen center point and WNT puncta using a custom macro. The angle of each line relative to the lumen center was recorded, summarized and averaged for all nephrons, binned and graphed in a radar plot using a custom Python script.

Quantification of spatial transcriptional data in the NPC population and PTA along an adjacent-distant axis: Spatial transcriptomics data from capturing a total of 9 CD tips and nephron progenitors with associated PTAs, from week 13 and week 16 kidneys, were analyzed for expression of PCDH15, MEOX1, ELAVL4, and FGF8. The distribution of transcripts was graphed in relation to the surface of each WNT11 CD tip. Pretubular aggregates were defined based on morphologies (DAPI) and the collective view offered by expression of PCDH15, MEOX1, ELAVL4, FGF8, WNT4, LHX1, JAG1, EMX2, GATA3, POU3F3, HNF1B, LEF1, PDGFRB, in relation to the WNT11-to-WNT9B transition in the CD. Transcript distribution was plotted in PTAs for WNT4, FGF8, LHX1, EMX2, JAG1, MEOX1, and PCDH15 with respect to the forming lumen in the PTA and the WNT11-to-WNT9B transition point. The angle of each line relative to the lumen center was recorded, summarized and averaged for all nephrons, binned and graphed in a radar plot using a custom Python script.

Quantification of morphogenesis: Organizer-coupled HNF4A::YFP organoids were imaged via widefield fluorescence microscopy on Day18 and processed on ImageJ/FiJi. The center of the mCherry+ organizer was marked with the point tool and its coordinates recorded; then, arrows were drawn from YFP+ tubules to YFP− tubules in clearly polarized nephrons showing elongated morphologies around the organizer, and the coordinates of the start and end of the arrow segment were recorded. Then, with a custom Python script, the distance between the end of the arrow, and the alignment angle between the morphogenetic field lines and the nephron PD axis were calculated per nephron and averaged per organoid (see FIG. 12G for diagram of measures).

Quantification of WNT-inhibited organizer-coupled organoids: Organizer-coupled HNF4A::YFP organoids treated for 48 hours with titrated IWP2 or titrated endo-IWR1 were imaged via widefield fluorescence microscopy on Day 14 and processed on ImageJ/FIJI. Individual YFP+ tubules were marked with the multipoint tool, and perpendicular diameters to represent major and minor axes of the mCherry+ organizer were drawn using the line tool. XY coordinates were extracted and an organizer XY centerpoint coordinate was calculated by intersecting the major and minor axes. Euclidian distances between each YFP+ point and the organizer center point were calculated and subsequently filtered to keep distances less than the organizer's radius+262.5 μm. Filtered distances across samples were analyzed in R.

Immunostaining and clearing: Kidney organoids and mouse kidney explants were fixed for 20 min on ice in 4% PFA. Samples were permeabilized and blocked using PBS with 1.5% SEA Block (Thermo Fisher Scientific, 37527X3) and 0.1% TritonX100 (EMD Millipore, 1.08643) at 4° C. with gentle movement for 1 hour. Samples were stained with primary antibodies in blocking solution overnight. Stained samples were washed for at least 3 hours through several rounds of PBS-0.1% TritonX100 washes and stained with secondary antibodies in blocking solution overnight. PBS washing steps were repeated the next day and nuclei were counterstained using PBS-0.1% TritonX100 containing 1 μg/mL Hoechst 33342 (Thermo Fisher Scientific, H3570) for 25 minutes. For widefield microscopy, samples were washed for 1 hour in PBS and mounted on slides with a glass coverslip in Immu-Mount (Thermo Fisher Scientific, 9990402). For confocal microscopy, organoids were cleared by gradual dehydration in and placed in BABB 16.

Confocal imaging and volume analysis: Optically cleared organoids were mounted in 20 mm-diameter FastWell frames (Grace Bio-Labs, #664113) loaded with 190 μl benzyl alcohol-benzyl benzoate (BABB). A coverslip shard was put on top of the transwell to ground the organoid, another round coverslip was used to seal the FastWell, and the sealed chamber was nail polish-glued to a Superfrost plus microscope slide. Organoids were imaged on a Leica SP8× confocal laser scanning microscope by stitching 20 Z-stack tiles captured using 40× oil lens (512×512 resolution). For thicker organoids a 25× water long-working distance lens was used. Imaging files extracted as TIFs were converted to IMS format using Imaris file converter and processed on Imaris imaging analysis software. Organizer surfaces were rendered from manually drawn regions on the basis of strong CDH3 or faint TFAP2A signal; a positive mask was extracted from the surface (organizer channel), and negative masks subtracting the organizer from all other channels were produced. Surfaces from resulting organizer-subtracted channels were rendered with a minimum 2000 μm³ volume threshold, and manually curated by deleting objects stemming from the mesenchyme, organizer leftover signal, and the autofluorescent transwell. Statistics and surface galleries were extracted via the Vantage tab. 3D renders were visualized using the Blend option and images captured via the Snapshot tab.

Statistics: The exact sample size (n) and statistic test employed for each dataset are detailed in the key numbers table. Repeated measurements were performed when different nephrons from the same organoid were quantified, when different immunostain intensities from the same nephron region were extracted, and when different metrics from the same image were extracted. Unless otherwise noted, data were assumed to be normally distributed. Significance and effect sizes are noted on figure panels (P: P-value, f.c.: fold-change, D: Cohen's d). Means and standard deviations are described in text. One-tailed t-tests were performed if the variable tested was predicted to exert a directional change.

To assess whether the distribution of alignment angles deviated from a uniform (random) distribution, the study applied the Rayleigh test for circular uniformity. For each condition (control and experimental), individual alignment angles were converted to radians and analyzed using a custom Python application of the known Rayleigh test formulation for circular data, implemented with NumPy vector operations following the CircStat MATLAB Toolbox. The test computes a Rayleigh Z-statistic, which quantifies the strength of unimodal clustering around a mean direction, and an associated p-value indicating the probability that the observed distribution differs from uniformity. Reported Z and p values reflect independent tests performed for each condition. A significance threshold of p<0.05 was used to reject the null hypothesis of uniformity.

Materials are shown in TABLE 1.

TABLE 1
Materials.

| | | |
|---|---|---|
| REAGENT or RESOURCE | SOURCE | IDENTIFIER |

Antibodies

| | | |
|---|---|---|
| EMX2 Polyclonal Sheep IgG | Biotechne | Cat#AF6470 |
| DLL1 monoclonal (251127) mouse IgG_{2B} | R&D Systems | Cat#MAB1818 |
| JAG1 Polyclonal Goat IgG | Novus | Cat#AF599 |
| LEF1 monoclonal (C18A7) Rabbit IgG | Cell Signaling | Cat#2286S |
| | Technologies | |
| HNF1B monoclonal (CL0374) mouse IgG₁ | Thermo Scientific | Cat#MA5- |
| | | 24605 |
| POU3F3 polyclonal goat IgG | Novus | Cat#NBP1- |
| | | 49872 |
| POU3F3 polyclonal rabbit IgG | Thermo Scientific | Cat#PA5- |
| | | 64311 |
| CDH1 monoclonal (36) mouse IgG2a, κ | BD Laboratories | Cat#610181 |
| ZO-1 monoclonal (ZO1-1A12) mouse IgG₁ | Thermo Scientific | Cat#33-9100 |
| TFAP2A monoclonal (3B5) mouse IgG_{2b} κ | Santa Cruz Biotechnology | Cat#sc-12726 |
| MAFB Monoclonal Mouse IgG2B | R&D Systems | Cat#MAB3810 |
| GATA3 polyclonal Goat IgG | R&D Systems | Cat#AF2605 |
| GFP polyclonal chicken IgY | abcam | Cat#ab13970 |
| WT1 monoclonal (CAN-R9(IHC)-56-2) | abcam | Cat#ab89901 |
| rabbit IgG | | |
| Donkey-anti-Rabbit IgG AlexaFluor488 | Thermo Scientific | Cat#A-21206 |
| Donkey-anti-Goat IgG AlexaFluor555 | Thermo Scientific | Cat#A-21432 |
| Donkey-anti-Rabbit IgG AlexaFluor647 | Thermo Scientific | Cat#A-31573 |
| Donkey-anti-Chicken IgY (IgG) | Jackson | Cat#703-545- |
| AlexaFluor488 | | 155 |
| Donkey-anti-Sheep IgG AlexaFluor488 | Jackson | Cat#713-545- |
| | | 147 |
| Donkey-anti-Rabbit IgG AlexaFluor555 | Abcam | Cat#ab150074 |
| Rat-anti-Mouse IgG_{2b} AlexaFluor647 | Abcam | Cat#ab172327 |
| Rat-anti-Mouse IgG₂ₐ AlexaFluor647 | BioLegend | Cat#407116 |
| Rat-anti-Mouse IgG₁ AlexaFluor647 | BioLegend | Cat#406618 |

Bacterial and virus strains

| | | |
|---|---|---|
| Escherichia coli HST08 strain (Stellar | Takara Bio | Cat#636763 |
| Competent Bacteria) | | |

Chemicals, peptides, and recombinant proteins

| | | |
|---|---|---|
| Doxycycline hydrochloride | Sigma Aldrich | Cat#D3447- |
| | | 500MG |
| Puromycin dihydrochloride | Thermo Scientific | Cat#A1113803 |
| Geneticin (G418 sulfate) | Thermo Scientific | Cat#11811023 |
| Y-27632 | STEMCELL Technologies | Cat#72304 |
| CHIR99021 | Tocris | Cat#4423 |
| FGF9 | Biotechne | Cat#273-F9- |
| | | 025 |
| Heparin | Sigma Aldrich | Cat#H4784 |
| Benzyl alcohol | Sigma Aldrich | Cat#402834- |
| | | 500ML |
| Benzyl benzoate | Sigma Aldrich | Cat#B6630-1L |
| endo-IWR1 | Tocris | Cat#3532 |
| IWP2 | Tocris | Cat#3533 |
| Dimethyl sulfoxide, ReagentPlus ®, ≥99.5% | Sigma Aldrich | Cat#D5879 |
| Wnt3a Alternative Peptide | PeptiGrowth | PG-008 |

Critical commercial assays

| | | |
|---|---|---|
| Spatial Genomics Custom probe set | This study | |
| Xenium Custom probe set | This study | |
| RNeasy Mini Kit | Qiagen | 74104 |
| Evercode Cell Fixation v2 | Parse Biosciences | ECF2001 |
| Evercode WT v2 | Parse Biosciences | ECW02030 |

Deposited data

| | | |
|---|---|---|
| hIPSC scRNA-seq day 18 (+/−organizer) | This study | |
| Organoid bulk RNA-seq days 10, 12 | This study | |
| Organoid bulk RNA-seq (+/−organizer) | This study | |
| Spatial transcriptional data (seqFISH) | This study | |
| Spatial transcriptional data (Xenium) | This study | |
| Human snRNA-seq weeks 15, 17 | Kim, S. et al. Comparative | GSE232479 |
| | single-cell analyses | |
| | identify shared and | |
| | divergent features of | |
| | human and mouse kidney | |
| | development. Dev Cell | |
| | (2024). | |
| Human scRNA-seq week 14 | Lindstrom, N. O. et al. | GSE139280 |
| | Spatial transcriptional | |
| | mapping of the human | |
| | nephrogenic program. Dev | |
| | Cell 56, 2381-2398.e6 | |
| | (2021). | |
| Human scRNA-seq week 17 | Tran, T. et al. In Vivo | GSE124472 |
| | Developmental | |
| | Trajectories of Human | |
| | Podocyte Inform In Vitro | |
| | Differentiation of | |
| | Pluripotent Stem Cell- | |
| | Derived Podocytes. Dev | |
| | Cell 50, 102-116.e6 | |
| | (2019). | |
| hIPSC scRNA-seq days 10, 12, 14 | Schnell, J. et al. | GSE264678 |
| | Controlling nephron | |
| | precursor differentiation to | |
| | generate proximal-biased | |
| | kidney organoids with | |
| | emerging maturity. Nat | |
| | Commun 16, 8136 (2025). | |
| Human bulk RNA-seq | Lindstrom, N. O. et al. | GSE100859 |
| | Conserved and divergent | |
| | features of human and | |
| | mouse kidney | |
| | organogenesis. Journal of | |
| | the American Society of | |
| | Nephrology 29, 785-805 | |
| | (2018). | |
| Organoid bulk RNA-seq days 10, 12 | Schnell, J. et al. | GSE264217 |
| | Controlling nephron | |
| | precursor differentiation to | |
| | generate proximal-biased | |
| | kidney organoids with | |
| | emerging maturity. Nat | |
| | Commun 16, 8136 (2025). | |

Experimental models: Cell lines

| | | |
|---|---|---|
| HNF4A^{YFP} | Vanslambrouck, J. M. et | |
| | al. A Toolbox to | |
| | Characterize Human | |
| | Induced Pluripotent Stem | |
| | Cell-Derived Kidney Cell | |
| | Types and Organoids. J | |
| | Am Soc Nephrol 30, | |
| | 1811-1823 (2019). | |
| FiPSC | | |
| WTC11 | Gladstone Institute/Bruce | |
| | Conklin | |
| T-REx-293 | ThermoFisher Scientific | Cat#R71007 |
| P-cells/HEK^{Cdh3} | Cachat, E. et al. 2- and 3- | |
| | dimensional synthetic | |
| | large-scale de novo | |
| | patterning by mammalian | |
| | cells through phase | |
| | separation. Sci Rep 6, | |
| | 20664 (2016). | |
| HEK^{Cdh3-Wnt3a} | Glykofrydis, F., et al. | |
| | Bioengineering Self- | |
| | Organizing Signaling | |
| | Centers to Control | |
| | Embryoid Body Pattern | |
| | Elaboration. ACS Synth | |
| | Biol 10, 1465-1480 | |
| | (2021). | |
| HEK^{mCherry-Cdh3} (R1C3mC) | This study | |
| HEK^{mCherry-Cdh3-diWnt3a} (R1C3mC; R26W3a) | This study | |
| HEK^{mCherry-Cdh3-diWNT9B} | This study | |
| (R1C3mC; R26W9B) | | |

Experimental models: Organisms/strains

| | |
|---|---|
| Mouse C57/B16 | JAX |

Oligonucleotides

| | | |
|---|---|---|
| SEQ ID NO: 1 | This study | N/A |
| SEQ ID NO: 2 | This study | N/A |
| SEQ ID NO: 3 | This study | N/A |
| SEQ ID NO: 4 | This study | N/A |
| SEQ ID NO: 5 | This study | N/A |
| SEQ ID NO: 6 | This study | N/A |
| SEQ ID NO: 7 | This study | N/A |
| SEQ ID NO: 8 | This study | N/A |
| SEQ ID NO: 9 | This study | N/A |
| SEQ ID NO: 10 | This study | N/A |
| SEQ ID NO: 11 | This study | N/A |
| SEQ ID NO: 12 | This study | N/A |
| SEQ ID NO: 13 | This study | N/A |
| SEQ ID NO: 14 | This study | N/A |
| SEQ ID NO: 15 | Aznauryan, E. et al. | N/A |
| | Discovery and validation | |
| | of human genomic safe | |
| | harbor sites for gene and | |
| | cell therapies. Cell reports | |
| | methods 2, 100154 (2022). | |
| SEQ ID NO: 16 | Aznauryan, E. et al. | N/A |
| | Discovery and validation | |
| | of human genomic safe | |
| | harbor sites for gene and | |
| | cell therapies. Cell reports | |
| | methods 2, 100154 (2022). | |
| SEQ ID NO: 17 | This study | N/A |
| SEQ ID NO: 18 | This study | N/A |
| SEQ ID NO: 19 | This study | N/A |
| SEQ ID NO: 20 | This study | N/A |
| SEQ ID NO: 21 | This study | N/A |
| SEQ ID NO: 22 | | N/A |
| SEQ ID NO: 23 | | N/A |
| SEQ ID NO: 24 | Ran, F. A. et al. Genome | N/A |
| | engineering using the | |
| | CRISPR-Cas9 system. Nat | |
| | Protoc 8, 2281-2308 | |
| | (2013). | |

Recombinant DNA

| | | |
|---|---|---|
| pSpCas9-GFP | Addgene | Cat#48138 |
| pSpCas9-ROGI1gRNA | This study | N/A |
| pSpCas9-ROSA26gRNA | This study | N/A |
| pHDR-ROGI1-MCS | This study | N/A |
| pHDR-ROGI1-mCherry^{Surface}-2A-Cdh3 | This study | N/A |
| pHDR-ROSA26-MCS | | N/A |
| pHDR-ROSA26-TagBFP-2A-Wnt3a | This study | N/A |
| pHDR-ROSA26-TagBFP-2A-WNT9B201 | This study | N/A |

Software and algorithms

| | |
|---|---|
| GraphPad Prism (version 9.0.0-10.2.0) | GraphPad |
| ImageJ/FiJi | Open source |
| Imaris (version 10.1.1) | Oxford Instruments |
| RStudio | Posit |
| Seurat v4 | |
| splitpipe (v1.0.6p) | Parse Biosciences |
| STAR Align (version 2.7.10a) | Dobin, A. et al. STAR: |
| | ultrafast universal RNA- |
| | seq aligner. Bioinformatics |
| | 29, 15-21 (2013). |

Other

| | | |
|---|---|---|
| FW20-FastWells 20 mm Diameter X | Grace Bio-Labs | Cat#664113 |
| 1.0 mm Depth | | |
| Micro Cover Glasses, Round, No. 1, | VWR | Cat#48380-080 |
| Diameter = 25 mm | | |
| Scintillation Vials, Borosilicate Glass | VWR | Cat#66022-081 |

### Example 2: Defining and Controlling Axial Nephron Patterning with Synthetic Wnt-Secreting Organizers

Current hPSC-derived kidney organoids contain nephron-like structures that lack patterning. It is thought that during normal development, nephrons form their proximal-distal axial polarity in response to collecting duct-derived signals that are absent in kidney organoids. To delineate how polarities emerge, human kidney development was profiled by spatial transcriptomic approaches and in the process describe a new axial polarity in the human nephron, while demonstrating that the nephron proximal-distal polarity forms adjacent to a transcriptional boundary in the collecting duct where non-canonical WNT11 is downregulated and canonical WNT9B ligand is upregulated. The nephron in turn activates a series of canonical WNT targets with known roles inferring positional nephron identities. To test if a canonical WNT source can imitate the in vivo state in kidney organoids, self-organizing WNT secreting synthetic organizers were bioengineered. Organizer-coupled kidney organoids respond to WNT ligands by forming expression gradients and developing distal cell identities. Tuning the WNT dose produced nephrons with continuous patterning along the proximal-distal axis. Strikingly, polarized nephrons directed their distal tubules towards the WNT-source, indicating both axial patterning and morphogenetic programs tuned by WNTs. These data present a strategy to control organ patterning, building an artificial kidney, and highlights the power of synthetic organizer systems for advancing organoid models.

Kidney organoids generated from induced pluripotent stem cells (iPSCs) represent a promising source of synthetic kidney tissue to solve the scarcity of donor kidneys available for transplantation. To reach clinical relevance, kidney organoids must generate patterned nephrons that filter blood in renal corpuscles, reabsorb electrolytes in proximal convoluted tubules, regulate water and concentrate urine in the loop-of-Henle and distal tubules draining into a collecting duct. However, current kidney organoids contain nephrons that display limited maturation of distal cell types, and incomplete proximal-distal (PD) patterning. This suggests that current organoid derivation methods can be modified to include key mechanisms and tissues responsible for nephron patterning and kidney development, steps that would broaden the potential of organoid nephrons as therapeutics.

Nephron patterning arises during kidney organogenesis. Kidneys develop from reciprocal interactions between the collecting duct (CD) and the metanephric mesenchyme that contains nephron progenitor cells (NPCs) and stromal populations. Throughout kidney development the CD and the forming nephron are closely connected (see FIG. 1A for a schematic). The developing CD includes molecularly distinct tips (WNT11B⁺) and stalk (WNT9B⁺) populations. NPCs marked by SIX2, CITED1, MEOX1, and PCDH15, are found surrounding CD tips and undergo balanced self-renewal and differentiation in response to CD stalk-derived WNT9B ligands that activate β-catenin signaling in NPCs to initiate nephrogenesis. Nephron formation is first seen as a stream of NPCs that exist their niche, downregulate SIX2 and condensate into pre-tubular aggregates (PTA; expressing WNT4, FGF8 and LHX1) under each CD tip. The PTA develops into a lumenized renal vesicle (RV) with polarized JAG1 and CDH1 expression towards the CD stalk as more NPCs are gradually recruited to the opposite end of the RV. The RV thereafter develops into a comma- and S-shaped body nephrons (CSB and SSBs, respectively) that adopt distal (POU3F3, TFAP2A, MECOM, SOX9, CDH1), medial (IRX1, JAG1), proximal (HNF4A) and podocyte (MAFB, WT1) segments; these identities reflect their relative time of arrival into the forming nephron. Studies in mice have shown that the distal nephron is marked by high Wnt/β-catenin activity and manipulation of β-catenin signaling leads to expansion of distal segments at the expense of medial and proximal segments. Given the relationship between the forming nephron and the CD tip/stalk, and Wnt and β-catenin components controlling distal nephron tubule development, it was therefore hypothesized that it acts as a signaling organizer. But whether it was the case in human developing kidney is not known.

Several kidney organoid models describe how to generate kidney cell-types by mimicking a developmental progression from pluripotency to metanephric mesenchyme-like cell states. Their strengths are in their relatively rapid timeline with nephrons forming between 10 and 15 days from cells leaving pluripotency, but off-target cell-populations are frequently reported, and they display limited maturation and function compared to adult nephrons. These models typically lack a CD epithelium which can be derived from an anterior intermediate mesoderm lineage through separate differentiation protocols. Instead, nephron differentiation is induced by pulsing the organoids with WNT/β-catenin agonist CHIR99021. In the Takasato protocol this occurs 3 days prior to the formation of nephron-like structures, and organoid nephrons therefore organize without a CD and its secretome.

Determining whether kidney organoids can be locally patterned by a CD-derived signals to generate patterned nephrons is therefore an important goal. Controlling patterning in other organoid models has been attempted with microfluidics delivery of growth factors and growth factors from cells. Recently, synthetic organizers have been used to pattern embryoid bodies (EBs) in mES cells and iPS cells derived brain organoids and these foundational studies show that delivery of growth factors from adjacent cell sources can initiate and influence patterning in regions of stem-cell-derived constructs. However, whether these approaches can finely pattern human organoids and mimic morphological and patterning features as seen in vivo, is not yet determined.

It was hypothesized that recapitulating the CD-to-nephron relationship in kidney organoids can normalize organoid nephron patterning. Here the spatial transcriptional landscape of the developing human kidney was first resolved and nephrons were shown to develop adjacent to the transition point from WNT11⁺ CD tips to WNT9B⁺ CD stalks. Nephron cells at this transition present with upregulated canonical WNT targets and connect to the CD epithelium to form a luminal connection that in the adult nephron enables drainage of urine into the CD. Detailing the nephron transcriptional profile demonstrates that nephrons form via an inherently asymmetric mechanism first developing a CD-adjacent to CD-distant axial polarity that presages nephron anatomies and formation of its known PD polarity. By performing comparative analyses between kidneys and organoids it is shown that organoids lack a canonical WNT signal input but respond to WNT-secreting synthetic organizer cells. Coupling organoid nephrons with organizers drives a canonical WNT response, promotes distal nephron early differentiation programs, and strikingly generates a morphoattractive response in nephrons to orients nephron tubules toward the WNT source. The data highlights a strategy to engineer axial asymmetry in kidney organoids, control differentiation programs, and pave the way to urine-draining artificial kidneys connected to an artificial epithelium.

### Results

Cross-lineage communication correlates with nephron polarization and morphogenesis in the human developing kidney: To scrutinize signaling interactions within the forming human nephron and its niche, parallel spatial transcriptional analyses was performed using Xenium and seqFISH mapping the expression of hundreds of genes at two stages of kidney development rich in nephron formation (FIG. 1, FIG. 2). The expression of known and putative WNT target genes in relationship to WNT ligands and markers of cell types present in the developing kidney was analyzed (FIG. 1A). Given that nephron development happens asynchronously and continuously across the developing kidney cortex, this allowed simultaneous observation of all stages of early nephrogenesis. These observations, described below, led to the hypothesis that the WNT expressing interface of the CD functions as a de facto organizer of the forming nephron during its aggregation and the formation of the nephron PD axial polarity (FIG. 1B).

The first stage of nephrogenesis, the PTA, is a cell aggregate which forms within 3 to 4 cell distances of the point where CD progenitors (WNT11⁺) transition into CD stalk (WNT9B⁺); marked with an asterisk in FIG. 1c. It has been previously shown that mesenchymal NPCs migrate and gradually integrate into the forming PTA and RV. Here, it was noticed that NPCs outside the PTA demonstrate polarized abundance of transcripts. An CD-adjacent stream of NPCs (ELAVL4^{high}/FGF8^{high}), and a CD-distant stream (ELAVL4^{low}/FGF8^{low}) can be distinguished compared to other NPC markers (PCDH15 and MEOX1) that are expressed equally in the CD-adjacent and CD-distant NPCs (FIG. 1D, FIG. 2B). The CD-adjacent stream connects to the PTA immediately next to the CD while the CD-distant stream connects to the PTA one cell layer away from the CD. The local maxima of FGF8 and ELAVL4 near the CD-epithelium, may indicate that their expression is induced in response to a polarized CD-derived signal (FIG. 1D). This distribution bias is evident in both weeks 13 and 16 (FIG. 1D). In week 13 kidneys the distribution is broader for ELAVL4 and FGF8, but still biased towards the CD compared to MEOX1 and PCDH15. The broader distribution is likely as a result of the thicker NPC layers present at week 13. Cells at the site of active recruitment in both streams (indicated as the end of the dashed arrows in FIG. 1C, right most spatial transcriptomic panel) are WNT4^{low}, forming further evidence for the PTA being asymmetric. These observations support a scenario by which the 2 streams of nephron progenitors form conveyor-belts of cells that set up an intrinsic asymmetry in the epithelializing nephron polarized along a ‘CD adjacent’ to ‘CD distant axis.

The PTA includes a group of cells morphologically distinct from NPCs. While the asymmetry in NPCs is subtle, the PTA demonstrates distinctly polarized gene expression depending on cells' position relative to the WNT11/WNT9B transition point. The cells of the PTA that are closes to the WNT11/WNT9B transition point and along the WNT9B⁺ CD-adjacent surface of the PTA upregulate WNT-p-catenin targets FGF8, LHX1, JAG1, EMX2, WNT4 while NPC marker genes PCDH15, and MEOX1 remain equal across the PTA (FIG. 1C-1D). These data indicate the PTA forms asymmetrically and contains cell subdivisions from the outset. PTAs progress to develop into RVs, epithelial pear-shaped cysts with an oblong distal domain extending in the direction of the elbow of the CD stalk. At this stage, the NPC stream appears as a single file of cells that connects with the proximal end of the RV, and the distal RV remains in tight association with the CD WNT11/WNT9B transition point (marked with an asterisk in FIG. 1E). While the distal-most region thus remains in a fixed position, gene expression-wise, the RV has evolved and patterned into a distal region (also known as connecting tubule, GATA3⁺), an expanding medial domain (JAG1⁺, HNF1B⁺), and a proximal domain (WT1⁺), where NPCs are recruited (FIG. 1E, FIG. 3C). The emerging connecting tubule domain and distal medial domains are now simultaneously patterned along their CD adjacent to CD distant and their PD axes, and the CD-adjacent RV surface displays polarized expression of WNT4, FGF8, LHX1, and EMX2 towards the WNT9B⁺ CD source (FIG. 1E).

To determine CD-adjacent to distant transcriptional asymmetry, RNA scope in situ hybridization was performed against WNT11, WNT9B, and WNT4 and immunolabeled KRT8 (marks CD and distal nephron) to define cell boundaries and map WNT4 expression in the RV in relation to the WNT11/WNT9B transition point. WNT4 transcripts were asymmetrically distributed towards the WNT9B⁺/KRT8⁺ CD stalk (FIG. 1F). Quantification of the spatial distribution of WNT4 puncta shows high WNT4 near the WNT9B source and low WNT4 in cells further from the WNT9B source at the site of NPC recruitment and the CD distant domain (FIG. 1F). These data confirm the CD distant-adjacent axis in the RV.

The data therefore suggest that WNT9B from the ureteric stalk acts as a short-range morphogen driving gene expression in NPCs, and the PTA and RV. As in situ hybridization and spatial transcriptomics mark mRNA regardless of the time when it was initially transcribed, it remains unresolved whether CD-distant distal WNT4 mRNA was generated earlier in the PTA stage when the cells are close to the CD WNT9B⁺ stalk or if low levels of WNT9B in the WNT11⁺ CD tips drive continuous expression.

After the initial asymmetry forms in the RV, it develops into the comma-shaped body (CSB) and thereafter the S-shaped body (SSB) nephron (FIGS. 1G-1H, FIG. 3D). The CSB nephron elongates along its PD axis and is a transitory stage to the more elaborately patterned SSB. CSB cells nearest the CD WNT11/WNT9B transition, now form the connecting tubule precursor domain, which is characterized by downregulation of WNT4 and FGF8, and expression of GATA3, POU3F3, and TFAP2A. While some WNT4 transcripts remain in those cells, the WNT4 expressing domain shifts proximally along the length of the CD-adjacent nephron abutting the WNT9B source. EMX2, TFAP2A, POU3F3, remain high, while genes marking later nephron precursor specification (IRX1, MAFB, WT1) are emerging or defining small subset cells in the medial, podocyte, and parietal epithelium. Other more mature markers such as PTPRO (podocyte) and HNF4A/G (proximal) have not yet been activated. As the nephron then transitions to the SSB, it adopts new morphological features of convolution, particularly the deep glomerular cleft but also the separation of the distal and medial domains. The SSB is characterized by WNT4 expression that now only persists in the region most closely adjacent to the WNT9B source. This domain is the HNF4A/G⁺ proximal precursor (pP) (FIG. 1H, FIG. 3E). Throughout this process, the nephron has remained defined by the WNT11/WNT9B transition point in the CD, to which it is now connected via a patent luminal connection. Several putative precursor of specific sub-population have developed by the SSB stage, marked by expression of (GATA3⁺/SALL3⁺/POU3F3⁺/TFAP2A⁺) in the connecting tubule precursors (pCNT), (GATA3^{low}/TFAP2A⁺) in distal precursors (pDT), (IRX1⁺/PAPPA2⁺) in loop of Henle and macula densa precursors (pLOH/pMD), proximal precursors (pP) (HNF4A/G⁺/HNF1B⁺), parietal epithelium precursors, and podocyte precursors (pPod) (MAFB⁺/PTPRO⁺).

To provide an independent assessment of the hypothesis that the WNT11/WNT9B junction functions as a point of reference and organization for early nephrogenesis, single-cell sequencing data (scRNA-seq) from kidneys was scrutinized at stages matched with the spatial transcriptional data (FIG. 6). Cell-types were resolved by iteratively clustering the nephrogenic and collecting duct lineages and identified the WNT11⁺/RET⁺ CD tip progenitors, WNT9B⁺/AQP2⁺ CD stalk populations, NPCs (CITED1⁺/MEOX1⁺), PTAs (WNT4⁺/PAX8⁺), and differentiating RV populations. Receptor-ligand interaction was queried via CellChat, which highlights pair-wise ligand/receptor interactions between cell populations (FIG. 6). These predict a canonical WNT ligand/receptor interaction from WNT9B expressing CD cells (highest in cd1) to PTA and RV cell populations (highest in n10 and n12) that reflect the anticipated spatial positions of these in the CD-adjacent PTA and RV as based on the spatial transcriptomic views. Based on these analyses, there is a collective view emerging predicting a canonical WNT interaction between WNT9B⁺ cells in the CD and WNT4⁺ cells in the PTA that is sustained in the RV stage.

These spatial and single cell transcriptomic analyses of human developing kidneys provide a unique view to the inherent asymmetric transcriptional landscape observed during nephron development and support the hypothesis that WNT9B⁺ CD cells function as spatial organizers for the forming nephron with a distal nephron program being initiated in the early nephron adjacent to the WNT9B source.

Human kidney organoids lacking ureteric epithelium contain non-polarized nephrons: To evaluate nephron patterning in organoids in the absence of a CD, time-course immunofluorescence and transcriptomic analyses of organoid nephrogenesis in a kidney iPSC-derived model adapted from the Takasato protocol, was performed (FIGS. 7A-7C, FIGS. 15A-15B).

Rosettes of WT1⁺, cyst-like structure reminiscent of PTAs are visible at day 10 (FIG. 7B), indicating that nephron precursors begin to epithelialize around day 10 (3 days after pulsing with GSK3-β inhibitor CHIR99021). The partially lumenized epithelial cell clusters are positive for Notch-ligand JAG1 and epithelial cadherin CDH1. Within 48 hrs of forming (day 12), they become fully lumenized epithelia which are morphologically reminiscent of RVs (FIG. 7B). Strikingly, the gene expression of these structures display a total radial symmetry in terms of gene expression, and no signs of polarized or gradual recruitment of NPCs; this is in opposition with what is observed in human kidney in utero development, where gene expression and NPC recruitment are polarized towards the CD WNT11/WNT9B transition point (compare FIG. 7B to FIG. 1E and FIG. 3C), WT1 and JAG1 markers in the second subpanel).

To assess organoid transcriptional profiles against the human developing kidney framework, bulk RNA sequencing was performed across days 10-12 and compared these to a range of in vivo time points (weeks 9, 13, and 17; DESeq2). Except for WNT3, other canonical WNTs (e.g., WNT9B and WNT3A) were expressed below reasonable transcript count threshold levels, as were nephron canonical WNT targets WNT4 and FGF8 (FIG. 7C). This is in contrast with the in vivo counterpart, where WNT9B and its targets WNT4 and FGF8 were readily detected. Organoids did, however, express WNT receptors and signaling transduction modifiers from the Frizzled, Low-density lipoprotein receptor-related protein receptors, and R-spondin family members (FZD1, FZD2, FZD4, FZD6, FZD7, FZD8, FZD9, FZD10, LRP5, LRP6, RSPO3, RSPO4—notably not RSPO1 or RSPO2) FIG. 8C. This suggests that organoid nephrons are ready to respond to canonical WNT ligands but do not do so because of the absence of the WNT secreting CD. Of note, there are differences in organoid and kidney expression profiles for non-canonical WNTs, and WNT11, WNT5A, and WNT5B were detected suggesting organoids only partially recapitulate the in vivo kidney state (FIG. 1C, FIG. 8C).

To validate the absence of a canonical WNT response during organoid nephrogenesis the PTA to RV transition is scrutinized when polarity would normally be predicted to arise, using single cell RNA sequencing data capturing days 10, 12 and 14 of organoid differentiation. WNT4 and FGF8 mRNA transcripts were absent in the organoid nephron (FIG. 15A-15B: data from recent work and in vivo data as shown in FIG. 1) and while a PTA-like cell state exists (low MEOX1, PCDH15, CITED1, and positive for EMX2, JAG1, LHX1, KRT8) it resembles a hybrid NPC-PTA cell state with undefined boundaries between NPC and PTA profiles (compare FIG. 7L, FIGS. 8A-8B) with in vivo gene expression plots FIG. 11 for MEOX1). Further, while ELAV4 is normally restricted to only the nephrogenic lineage, a PAX2⁻, PDGFRB⁺, ELVAL4⁺ interstitial cell is seen in organoids (FIG. 8B), thus representing a state not seen in vivo.

In vivo, JAG1 and EMX2 expression are thought to be downstream of the CD-secreted canonical WNT signal. In organoids, even in the absence of a canonical WNT, JAG1 and EMX2 transcripts rose ~3-fold between days 10 and 12 (FIG. 7B-7C, FIG. 8B). At day 14, genes marking distal nephron cells (e.g. POU3F3) were detected, but a lack of GATA3 transcripts indicates minimal specification of a bonafide connecting tubule (CNT), a key distal nephron derivative (FIG. 8B).

These findings highlight a model where kidney organoids contain NPC-like cells that undergo differentiation, epithelialization, and upregulation of JAG1 in the absence of polarizing ureteric stalk-derived WNT9B, leading to RV-like structures devoid of WNT4 and FGF8 expression and polarity.

Polarizing WNT-secreting signaling centers drive β-catenin target genes in organoid nephrons: Given that nephron cells that emerge in organoids possess canonical WNT pathway components, it was hypothesized that their lack of WNT target gene activation is to be imputed to a lack of canonical WNT ligands. To test this hypothesis, WNT-secreting synthetic organizer (SO) cell clusters were generated and validated. The SOs are composed by HEK cells that are genetically engineered to produce either a control fluorescent protein, or Wnt3a in a constitutive (Wnt3A-SO) or in a small-molecule tunable manner (iWnt3a-SO). All SO cells also express Cdh3 for increased self-aggregation, and mCherry as fluorescence marker (FIG. 7F). Of note, neither Wnt3A-SO nor SO cells endogenously express any canonical WNTs as confirmed by bulk RNA sequencing on the organizer cells themselves. To confirm that these cell clusters can elicit nephrogenic and canonical WNT responses, small (5,000 cells) Wnt3a-SO and control SO were assembled in ultra-low adhesion wells for 24h, and then positioned adjacent to nephron-forming niches of embryonic mouse kidney explants. As shown in FIGS. 9A-9B, ectopic Jag1⁺/Cdh1⁺ nephrons formed in response to Wnt3a-SO but not in SO controls, confirming that Wnt ligands produced by synthetic organizer cells can induce canonical Wnt-response in NPCs in explanted developing mouse kidney. To determine if nephron polarization can be manipulated in human iPSC-derived nephrons organoids, Wnt3A-SO and control SO were introduced on day 12, at a stage when nephrons are non-polarized RV-like structures and waited 2 days for signaling to occur before performing transcriptomic profiling of those nephrons closest to the SOs at differentiation day 14 (FIGS. 7F, FIG. 7M). To profile only organoid cells closest to the organizers, the region of cells in close proximity to the organizers were mechanically dissected and SO cells were removed based on negative FACS sorting for mCherry. As shown in FIG. 7M, exposure to Wnt3a-SO increased expression of EMX2, FGF8, and DLL1. WNT4 transcript counts remained at sub-threshold levels with a trend increase to Wnt3A-SO, likely due to WNT4 being expressed at low levels in general and diluting effect of other interstitial cell populations. Of note, DKK1 was increased in response to Wnt3a but neither distal gene POU3F3 nor GATA3 (distal patterning genes) responded to the ectopic Wnt3a ligands within the 48 hours timeframe. This is consistent with the in vivo setting where EMX2 and FGF8 emerge prior to POU3F3 and GATA3 adjacent to the WNT11/WNT9B transition point (compare to FIG. 1C).

Markers for the early CD-adjacent (JAG1) and podocyte differentiation cell states (WT1)) alongside epithelial marker CDH1 were assessed via IF in organoids conjugated with control and Wnt3a-SO. As shown in FIG. 7G, nephrons under and adjacent to control synthetic organizer are unaffected compared to neighboring structures but in Wnt3a-conjugated organoids, nephrons under and adjacent to Wnt3A-SOs grow larger and downregulate WT1 while upregulating JAG1 (FIGS. 1I-1J). This is consistent with previous work detailing β-catenin acting as an antagonist to proximal and podocyte development in mice. It is also noticed that the patterning effects were local and affected only nephrons in the vicinity of the organizer, but not regions of the organoid farther away from the organizer. To quantify the range of affected cells in the organizer, β-catenin target LEF1 protein levels were assessed, and observed increased LEF1 protein abundance up to hundreds of microns away from the organizer cells (FIGS. 9D-9E).

These results demonstrate that ectopic cellular signaling centers can express a canonical Wnt ligand that drives a β-catenin mediated response in human kidney organoid cells, locally program nephrons to a upregulate early distal cell markers (EMX2) and introduce developmental programs otherwise missing in the iPSC-organoid cultures.

Tunable WNT-secreting synthetic organizers drive axial symmetry breaking and morphogenesis: Building on these results, HEK organizer cells were engineered with tunable control of canonical Wnt ligands Wnt3a and WNT9B, reasoning that adjustable control of the secretion of Wnt ligands from organizer cells could allow the regulation of patterning in the organoids. To do so, new organizers producing Wnt ligands (Wnt3a and WNT9B) under a dox-inducible promoter, alongside constitutive expression of cadherin proteins (CDH1) were produced (FIG. 10D). Inducible WNT SO are from here on referred to as either iWnt3a-SO and iWNT9B-SO. Flow cytometry analyses showed a sigmoid dose-response curve of inducible expression of TagBFP-2A-Wnt3a transgene iWnt3a-SO cells (EC₅₀ of 2.14±0.74 nM), peaking past 10 nM (FIGS. 10A-10C), supporting capacity of the system to express transgenes in graded manner. To test the capacity of these iWnt-SO cells to induce WNT/β-catenin transcriptional responses in neighboring cells, co-cultures of iWnt-SO cells with mouse ES reporter cells carrying a β-catenin TCF/LEF-GFP reporter, were setup. As shown in FIG. 11A, the iWnt3a cells when induced with dox to express Wnt3a ligand, drive reporter activation in neighboring mES cells (see also FIG. 10E). When a similar experiment was performed with iWNT9B-SO cells, no activation of the reporter ES cells was observed. Given that the co-receptor RSPO1 can be important for WNT signaling downstream of certain ligands, it was tested if adding RSPO1-conditioned media in a co-culture experiment of iWNT9B and TCF/LEF mES cell reporters changed the outcome of the experiment. In this condition, robust ES cell reporter activation was observed, demonstrating capacity of iWNT9B cells to induce WNT/β-catenin responses in neighboring cells in presence of RSPO-1 (FIG. 11B and FIG. 10F). Given the difference between Wnt3a and WNT9B results, the capacity of iWNT9B-SO to induce tunable nephrogenesis in mouse kidney explants was further tested. To do so iWNT9B-SO were conjugated to mouse kidney explant in presence of increasing doses of dox. As shown in FIG. 10H, differentiation of Jag1⁺ nephron precursors is induced adjacent to the iWNT9B organizers in a dose-dependent fashion. Of note, in the kidney explant WNT9B acts without external supplementation of RSPO-1, as the co-receptor is likely already present in vivo.

With these validated, tunable iWnt-SO, it was tested if they drive patterning in human kidney organoids. To do so, kidney organoids were coupled with iWnt-SO at day 12 (when they begin to split their PD axes), using varying amounts of dox, and profiled nephron axial polarities after 48 hours by immunofluorescent stains for nephron patterning markers POU3F3 (distal), JAG1 (medial), and WT1 (proximal) as well as EMX2 (distal transcription factor) and DLL1 (medial/proximal Notch ligand); the latter two chosen as they were sensitive markers for cells responding to constitutive Wnt3a (FIG. 7M). As shown in FIG. 11C, iWnt3a organizers drive a biphasic, dosage-dependent upregulation of WNT/β-catenin and nephron distal patterning markers EMX2 and POU3F3 and induce several morphological changes. To describe these responses, the patterning of EMX2 and POU3F3 and the length of the nephrons were assayed by measuring the polarization of markers within individual nephron tubules relative to the position of the organizer (in vivo nephron axial asymmetry was established in FIG. 1). As shown in FIGS. 11D-11E, FIGS. 10H-10K, the position of these distal markers is enriched in the part of the nephron that is the closest to the organizer, the nephrons elongate towards the Wnt3a source, and the total number of nephrons that are polarized increase. DLL1 was similarly upregulated in a dosage dependent manner, whereas proximal marker WT1 was downregulated (FIGS. 11G-11I).

The study also set up similar organoid conjugation experiments with iWNT9B-SO. Given the requirement of iWNT9B organizers for RSPO-1 for stem-cell inductions, a WNT9B/RSPO-1 matrix of culture condition was set up to expose organoid nephrons to dosages of each. As shown in FIGS. 11J-11L, and similarly to iWnt3a results, overall presence of both RSPO-1 and WNT9B resulted in induction of DLL1 and JAG1, and downregulation of proximal marker WT1. In these experiments, it was noticed that nephrons were highly sensitive to changes in WNT9B and RSPO-1: DLL1 is virtually absent in organoids where the WNT9B is not induced (0 nM dox column), but becomes strongly upregulated in 5 nM dox WNT9B induction at any RSPO-1 dosage. A higher DLL1 increase was observed at 5 nM dox with 100 ng/mL RSPO-1 compared to the 50 nM dox equivalent, an effect lost at the 500 ng/mL RSPO-1 dose. This suggests that nephrons respond in dosage-dependent manners to WNT ligand inputs.

These results show that iWnt-SO are able to drive dose-dependent WNT/β-catenin signaling in iPS C-derived nephrons which lead to patterning nephrons along the proximo-distal axis and align towards the synthetic organizers.

WNT-secreting synthetic organizers orchestrate patterning and morphogenetic events in nephrons: It has been shown that the initial events of patterning and morphogenesis of nephron development can be controlled by WNT producing synthetic organizers. It was therefore asked if and how a continuous exposure to Wnt ligands, like the one that is observed in vivo (see FIG. 1), affects nephron patterning and morphogenesis in vitro.

To do so, first constitutively expressing Wnt3a synthetic organizers were conjugated with kidney organoids at day 12, stained, confocally scanned and 3D reconstructed coupled organoids at day 18. To assess distal differentiation, kidney organoids were labeled with markers CDH1 and POU3F3 which label epithelial cells of the distal nephron, and tight junction protein ZO-1 which marks the apical surface throughout the epithelial component of the nephron (distal and proximal) and strongly labels apical surfaces of proximal renal corpuscle cells (podocyte and parietal epithelium). In control SO-conjugated organoids, nephrons positioned under the organizers developed normal renal corpuscles, marked by strong apical ZO-1, as well as short POU3F3⁺ tubules, similarly to other regions of the organoids, distant from the organizer. Exposure to Wnt3a-SO instead transformed nephrons adjacent and under the organizer into distalized structures, devoid of renal corpuscles-like structures and enriched in distal structures (FIG. 12A and FIG. 13A). To perform quantitative analysis of this phenotype, 3 regions of the organoid were distinguished according to their distance to the organizer and their position relative to the rest of the organoid: an “under” region that is the closest to the organizer; an “in” region that is farther from the organizer towards the center of the organoid, and an “out” region that is at the same distance from the organizer as the “in” region but in the direction towards the periphery of the organoid (marked on FIG. 12A). POU3F3⁺ structures were categorized and counted as total POU3F3⁺ tubules, small truncated POU3F3⁺ cell clusters, and strongly positive ZO-1 renal corpuscles. Nephrons forming under the Wnt3a-SO did not develop renal corpuscles and instead generated POU3F3⁺/CDH1⁺ tubules, contrary to control-SO conjugated nephrons that develop normal renal corpuscles in that same region (FIG. 12B).

The absence of renal corpuscle (podocyte and parietal epithelium) cell fates in response to Wnt3a together with the distance-dependent abundance of LEFT (FIG. 9D) suggest nephrons exhibit a Wnt3a dosage response that is mapped out differently at different distances to the organizer. To scrutinize this, the spatial distribution of renal corpuscles developing was measured with respect to the organizer cluster boundary. In Wnt3a-SO conjugated organoids, nephrons developed renal corpuscles 108 μm from the organizer, as measured on the “inside” region, while this distance increased to 251 μm on the “outside” region (FIG. 12D). In control SO-conjugated organoids, these distances were considerably shorter at 35.7 μm and 39.1 μm, respectively. Nephrons in the “under” and “out” regions of Wnt3a-SO conjugated organoids were more susceptible to this effect (FIG. 12D). In the presence of Wnt3a, renal corpuscle cell fates were completely suppressed within a 100 um radius of the Wnt3a-SO on the outside region. Conversely, distal POU3F3 segments were larger and were distributed more towards Wnt3a-SO compared to controls-SO conjugated organoids (FIG. 12E). To confirm if these were changes in patterning of more than one marker genes, a similar panel of spatial distribution analyses was performed for proximal (HNF4A⁺) and distal (TFAP2A⁺) nephron segments, marking podocytes (WT1⁺) for reference (FIGS. 13A-13E). Similar to POU3F3 analyses, large distal TFAP2A⁺ segments were enriched adjacent to Wnt3a-SO compared to control-SO (FIGS. 13B-13C), while HNF4A⁺ segments objects were depleted and smaller; no HNF4A⁺ segments above 2×10³ μm³ were found within a 70-80 μm range from Wnt-sources while being abundant in controls (FIGS. 13D-13E). These data demonstrated a local effect of patterning of the nephrons in the region close to the organizers.

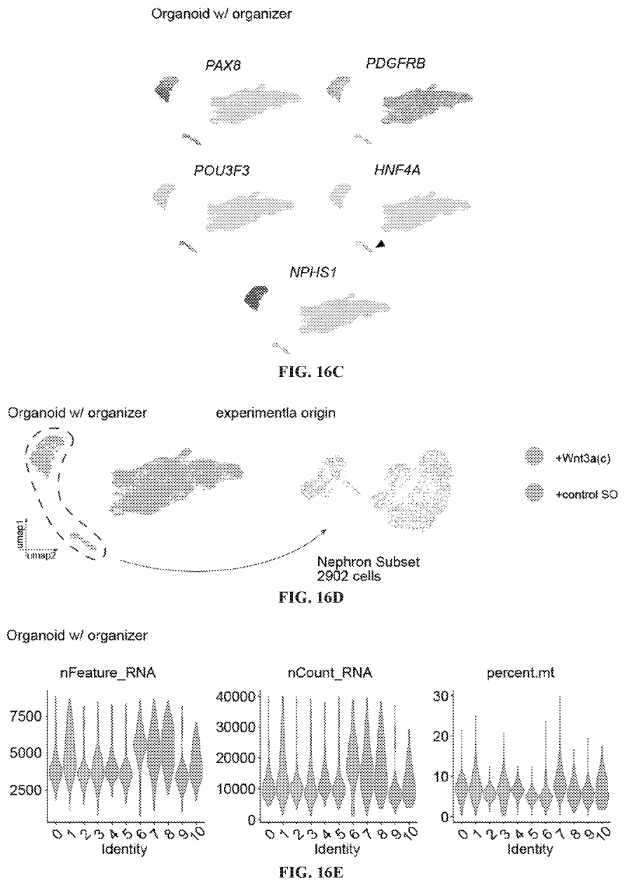

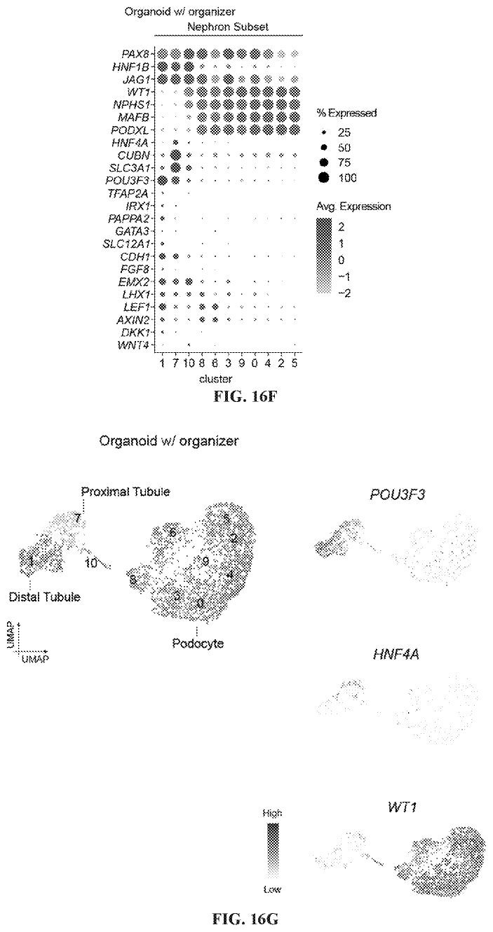

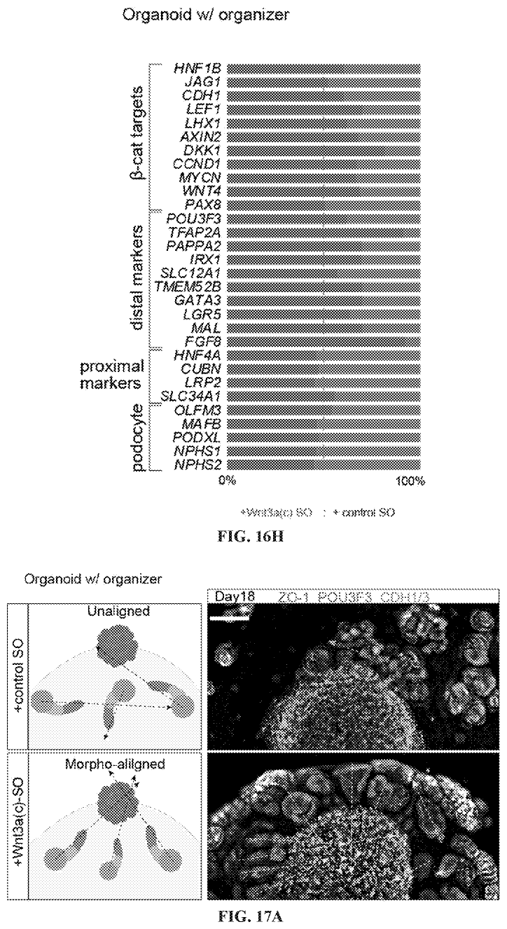

To corroborate patterning changes driven by Wnt3a in developing nephrons, spatially restricted single cell RNA sequencing (scRNA-seq) from Wnt3a-SO and control-SO coupled organoids was performed. Following cell clustering analyses, cell groups reflecting distal (POU3F3), proximal (HNF4A), and podocytes (NPHS1) identities were determined (FIGS. 16A-16G). Transcript reads for WNT/β-catenin-target genes known to be expressed in nephrogenesis (e.g., LEF1, AXIN2, DKK1, HNF1B, FGF8) were more frequently detected in nephron cells next to Wnt3a-SO than control-SO. Distal tubule markers (LGR5, POU3F3, TFAP2A), loop-of-Henle and macula densa (IRX1, PAPPA2, SLC12A1, FGF8), and connecting tubule (GATA3) were similarly enriched in Wnt3a-SO samples, whereas proximal (HNF4A) and podocyte markers (WT1, MAFB, PODXL) were minimally downregulated (FIG. 16H). The relatively high abundance of podocyte and proximal markers likely reflects the short distance that these cell identities were suppressed on the “inside” organoid facing surface (FIG. 12A) and the dissection made to isolate cells (FIG. 13A).

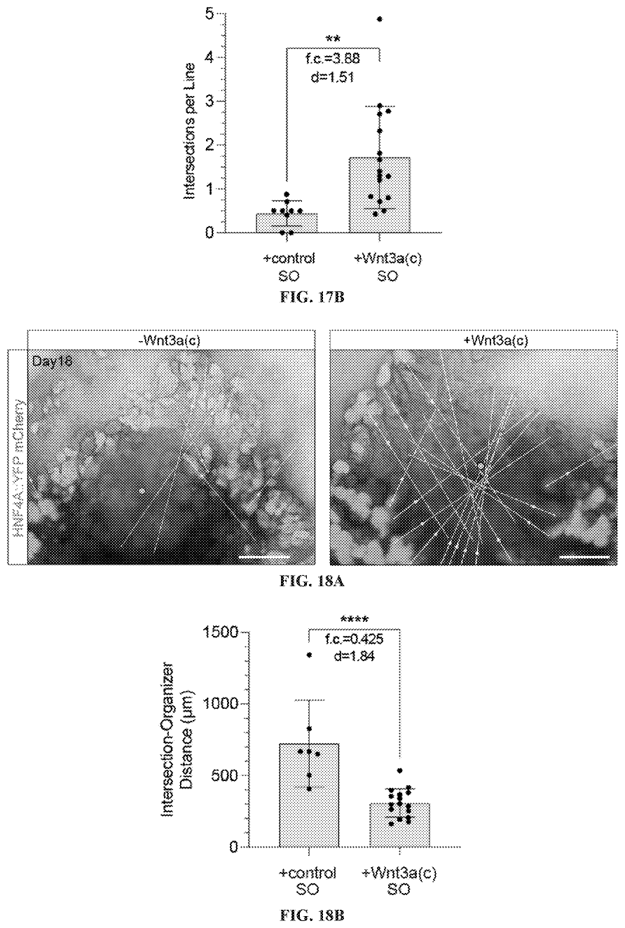

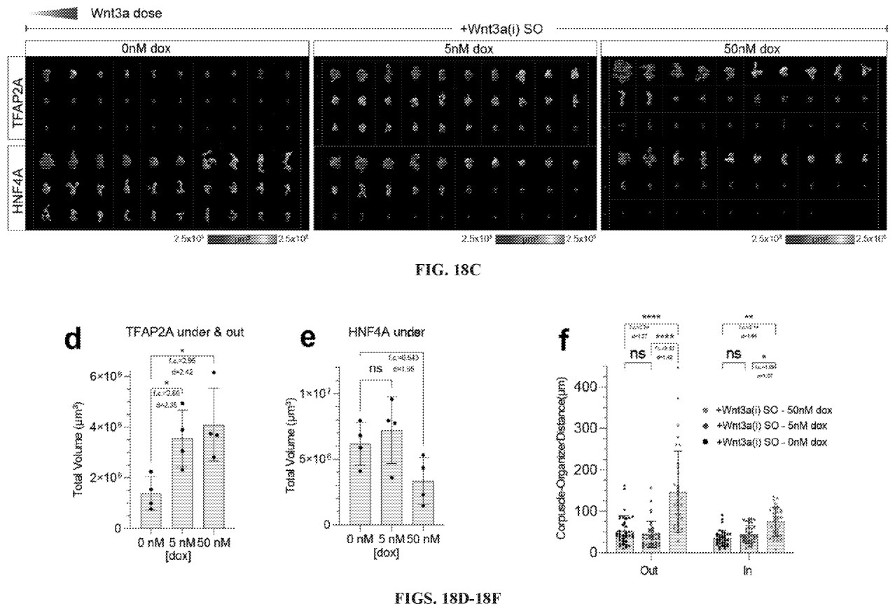

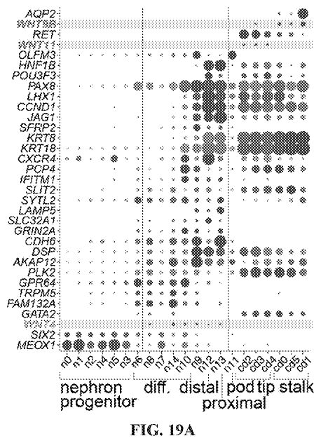

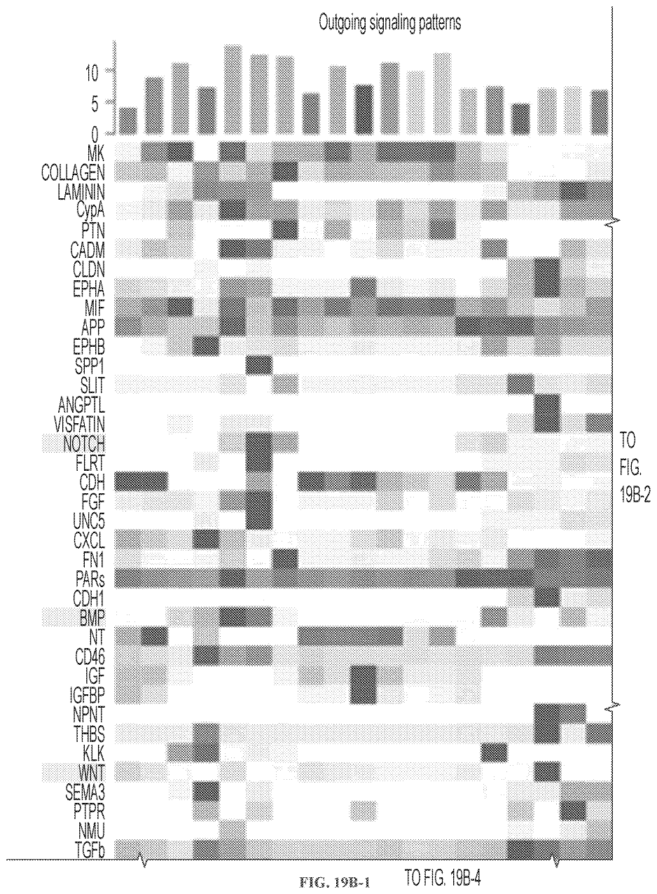

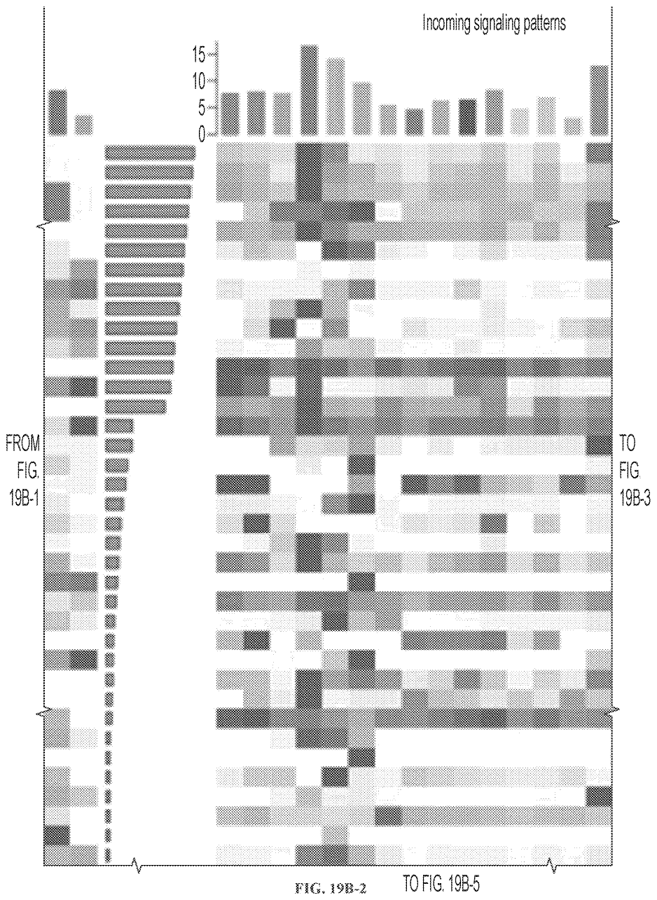

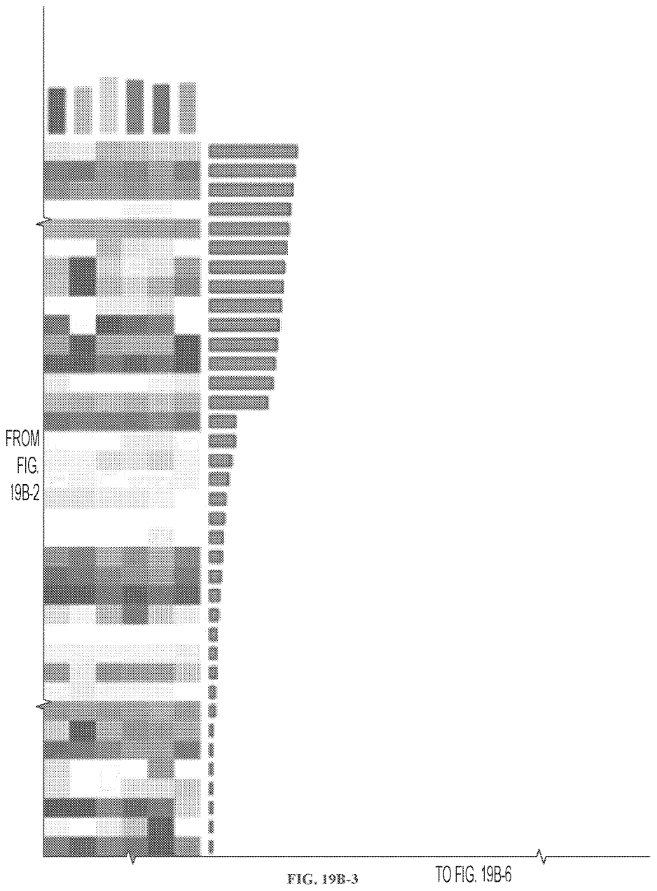

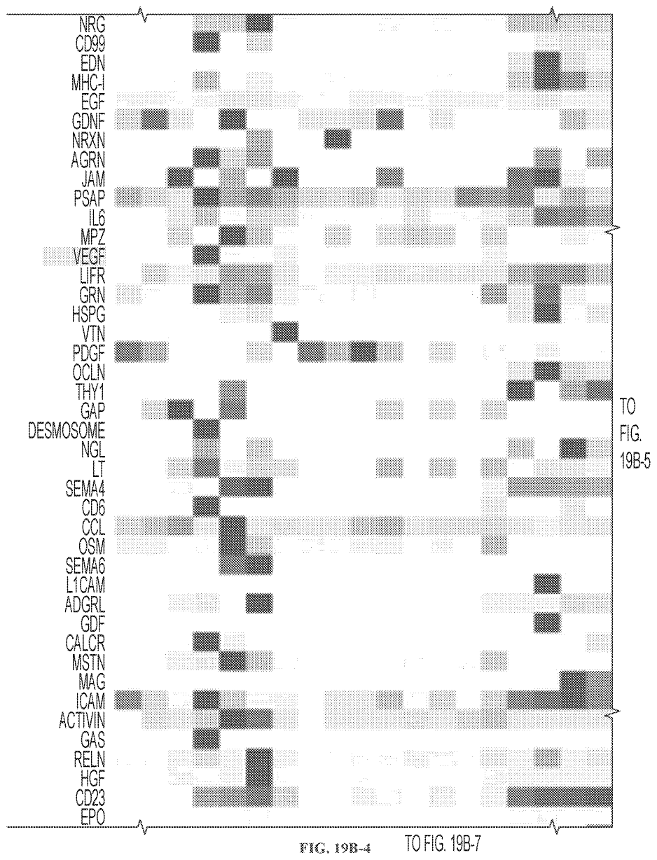

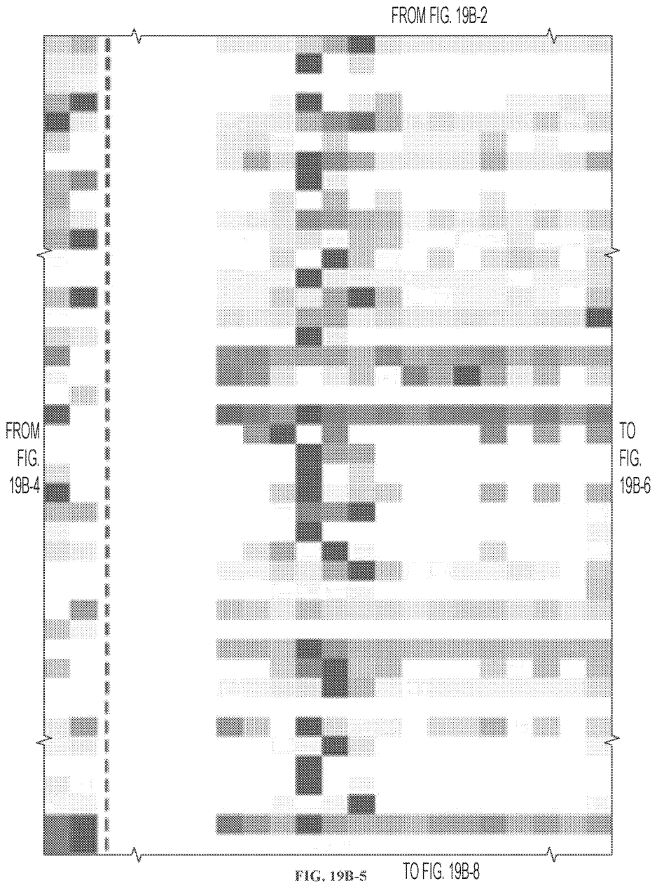

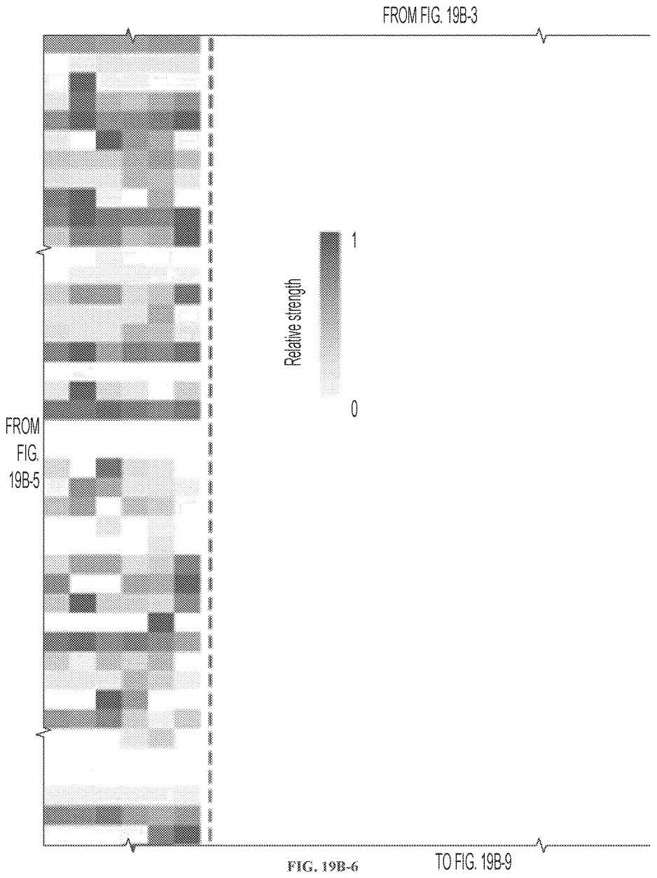

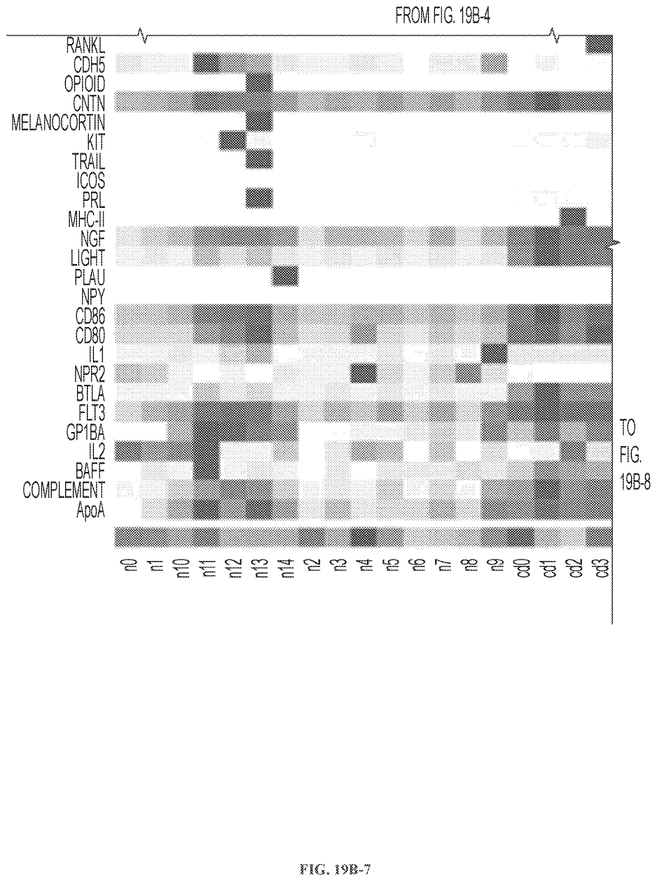

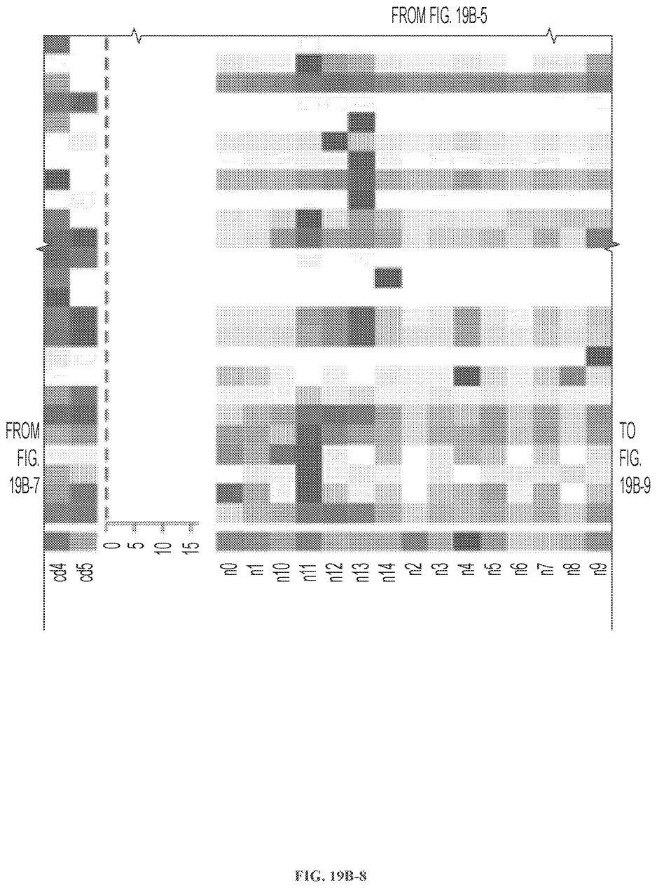

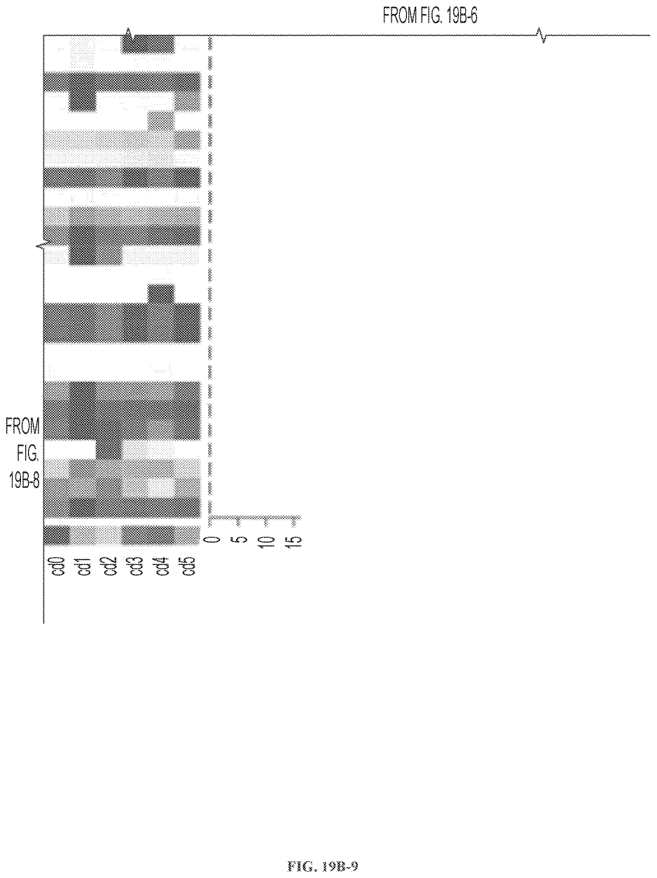

Analysis was then focused on the morphological changes associated with the developing nephrons. One first analysis focused on the volumetric transformation of nephrons. To do so, a collection of 3D-rendered POU3F3⁺ objects were generated. As shown in FIG. 12A (bottom row) and FIG. 12F (top row), these tubules' shape and seem larger in Wnt3a-conjugated nephrons compared to control-SO nephrons. To quantify this, the volume of these objects developing in the “out” and “under” region of Wnt3a-SO conjugated organoids was calculated and reported that they displayed a 2.26-fold increased volume of POU3F3 tubules compared to controls (μ=11.8×10⁶, σ=0.839×10⁶ μm³ vs. μ=5.24×10⁶, σ=1.42×10⁶ μm³, p<0.0001 FIG. 12C). Some nephrons in Wnt3a-SO conditions developed a particularly interesting phenotype, reminiscent of elongating distal tubule structures observed in vivo. Categorizing these as phenotype B in contrast to the shorter nephron phenotype A, phenotype B was defined as within a volumetric bracket (see methods). Phenotype B structures were found to be forming near Wnt3a-SO with 2.14-fold increased abundance compared to control organizers (FIG. 12F). Nephrons align towards the WNT11/WNT9B transition point in vivo, how they also aligned in the tunable iWnt-SOs (FIG. 3). This was also evident in the day 18 elongated nephrons. To characterize the alignment of the nephron structure towards the organizer the orientation of tubules relative to organizer was quantified (FIGS. 17A-17B). The orientation of tubules was defined based on their PD axes, using both immunolabelling and fluorescent reporter iPSC lines (FIG. 17A and FIG. 18A); the convergence of the tubule orientation path compared to the organizers was quantified (FIG. 17B, FIG. 18B). The projected paths of nephron tubules converged more frequently and closer to Wnt3a-SO compared to controls-SO, demonstrating that nephron tubules aligned their PD axes towards the WNT source.

These data show that Wnt3a secreting organizers drive a distal nephron program that suppresses proximal, renal corpuscle cell fates. Further, organoid nephrons orient their distal tubules towards the Wnt3a-SO and develop distal nephron cell identities in a distance-dependent manner, at the expense of proximal tubule and podocyte identities. Morphogenesis is affected by Wnt3a-SO, such that the orientation of the entire nephron morphological and patterning structure is aligned towards the organizer.

Exposure to high levels of Wnt3a expanded distal segments but at the expense of proximal identities. To determine if tuned Wnt3a sources can pattern nephrons without suppressing proximal and podocyte identities, TFAP2A (distal), HNF4A (proximal) and WT1 (podocyte) nephron segment sizes in day 18 organoids coupled with iWNT3a-SO were evaluated. 3D reconstruction of confocally scanned nephrons show normal WT1⁺ renal corpuscles and elongated HNF4A⁺ proximal precursor segments underneath iWNT3a-SO in the absence of doxycycline (FIG. 12J and FIG. 18C). At intermediate Wnt dosages (5 nM), continuity of TFAP2A-HNF4A-WT1 segments could be seen in nephrons just adjacent to the organizer and towards the organoid's periphery, while prominent TFAP2A⁺ distal nephron segments and renal corpuscles formed underneath in the absence of elongated HNF4A⁺ segments. At peak Wnt dosages (50 nM), prominent TFAP2A+ structures similarly appeared underneath organizers, but WT1 expression was sporadic and HNF4A was excluded to positions further from the organizer (FIG. 12K). To quantify these observations, total volume of TFAP2A and HNF4A segments were measured as shown in FIG. 18C, distal segments (TFAP2A⁺) increase in volume at increasing Wnt doses while correspondingly, HNF4A segment volumes decrease at the highest dose of Wnt (FIGS. 18D-18E). At high Wnt3a doses the tunable system mirrored the distalization and suppression of proximal identities observed earlier (FIG. 12A). Strikingly, at intermediate Wnt3a doses, both TFAP2A⁺ and HNF4A⁺ segments formed that connected within single nephron tubules (FIG. 12M). Moreover, the restriction of big distal and proximal segments to positions close to the organizer followed a dose-response curve at increasing doses of Wnt (FIG. 12K), and similarly for the restriction of corpuscles to regions farther from the organizer (FIG. 18F). This is consistent with distal differentiation being driven near the Wnt source and at a high dose but suggests an intermediate Wnt dose is compatible with a fully patterned nephron (FIGS. 12K-12M). As in FIG. 12A, it was also noticed that the distal domains seem to be directed towards the organizer and make contact with it.

Collectively, these experiments demonstrate that canonical WNT ligands can drive development of distal nephron segments without ablating proximal tubules and renal corpuscles and polarize nephrons towards the WNT source (FIG. 12J).

### Discussion

Spatial transcriptional analyses highlight a new axial polarity during nephron development: These findings describe an axial polarity detailing nephron cells' relationship to the CD. This is configured as NPCs positioned around the CD tip undergo a mesenchymal to epithelial transition to generate nephrons and thereafter the axis persists through the stereotyped developmental morphogenetic steps of the PTA, RV, CSB, and SSB impacting adult nephron morphologies and function. The spatial transcriptional analyses show that a CD adjacent-distant (AD) polarity precedes the formation of the PD polarity. The AD polarity initially traverses the partially epithelialized PTA and can be defined in the PTA by how cells are positioned in relation to the WNT11/WNT9B transition point and the WNT9B⁺ CD surface (shown in FIG. 1). Subsequent to PTA stages, the CD-adjacent domains define the nephron epithelium that is adjacent to the WNT9B⁺ CD stalk as the RV and CSB grows and elongate and the SSB folds into its typical structure. Adding the AD axis as a reference point to describe nephrogenesis assists in defining gene expression patterns and areas of the forming nephron and it integrates well with the proposed gradual recruitment model and a time-dependent mechanism dictating nephron fate specification through sequential activation of signaling pathways.

Providing a new term to an existing language model creates discussion on whether current nomenclature is sufficient. It is disclosed that the AD axis is welcomed as the kidney organoid field tackles questions on how to replicate stereotyped developmental steps in vitro. Further, it assists with changing misconceptions about the PTA. The PTA has long been described as entirely non-epithelial, but its nature is nuanced and includes partially epithelial cells in CD adjacent positions that epithelialize and asymmetrically upregulate gene expression while new NPC recruits are slotted into the forming nephron; a process intrinsically linked to cell recruitment and gradual formation of spatial positional identities. Given that this asymmetry plays a crucial role in nephron patterning and therefore acquisition of physiologies, it is necessary to be considered.

The data shows that molecularly distinct AD regions are prominent in the RV, CSB, and SSB, thus making it hard to envisage how SSB and subsequent nephron patterning are not influenced by this polarity. Putative precursors can be defined by it. For instance, in the SSB a secondary upregulation of WNT4 was detected in CD-adjacent proximal precursors. However, the dynamics of WNT4 illustrates the asymmetry of the nephron and puts forward an explanation for why podocytes are mosaically labelled with Wnt4^{Cre} fate mapping. Low expression of WNT4 in CD-distant cells is a likely reason for previously observed incomplete Wnt4^{Cre} mediate recombination in CD-distant podocytes, while other nephron segments are robustly labelled with Cre-dependent reporters. An AD axis also begins to shed light on the chirality of SSBs, as one axis is insufficient, but two axes can define chiral structures.

The perhaps most clear reason for why an AD axis is required can be demonstrated by a simple visualization. Picture the SSB, rotate it on its AD axis, and imagine how it now connects with the CD and forms podocytes in close contact with a developing macula densa—a hard feat. It is now needed to define AD fate-maps, better understand AD spatial transcriptional signatures in three dimensions, and determine the downstream functional significance of this interesting axis.

Building an artificial kidney: A cell derived kidney suitable for replacement therapies should be connected to a vasculature in and output, contain a physical filter, perform solute reabsorption, and lead unwanted solutes and metabolites to a drainage system. The latter function is normally performed by a luminal connection between the distal end of the nephron and the CD. It was therefore sought to delineate how the distal nephron is polarized towards the CD and replicate this in the organoid model with a view to develop an approach for channeling urine in an artificial kidney.

To control nephron patterning in hPSC kidney organoids WNT-secreting cellular organizers were introduced to replace CD-derived WNT9B ligands. This drove distalization and physical alignment of nephron towards the WNT source. Rationale was lent to these experiments from the in vivo spatial transcriptional analyses (seqFISH and Xenium) which show that β-catenin mediated transcription is polarized to those nephron cells adjacent to the WNT9B source. Human orthologs of previously proposed canonical WNT targets e.g., Emx2, FGF8, Jag1, Lhx1, Gata3, Wnt4, and Lef1 are expressed in that domain. These in vivo human data agree with strong evidence for crosstalk between the CD and nephron derived from mouse studies. In mice, WNT9B is produced in the CD and genetic studies show it is required in the nephron lineage, mediating NPC self-renewal, differentiation, and nephron formation, as well as for the maintenance of nephron planar cell polarity in maturing nephron tubules. In past work it has been shown that manipulation of the early mouse nephron with Wnt agonists or antagonists distalizes and proximalizes nephrons, respectively, which is in line with evidence from the chick mesonephros, where mesonephric tubules suppress podocyte development and reorienting nephron PD axes in response to Wnt3a expressed in the chick embryo. Further, these nephron patterning effects can at least partially be mimicked in hPSC organoids through positive and negative regulation of β-catenin signaling. The spatial transcriptional data therefore confirm that this WNT9B to nephron cross-lineage signal transduction is likely in the human nephrogenic niche and overcome past limitations of predictions made from single cell RNA sequencing data where spatial data has been missing.

Providing organoid nephrons with WNT secreting organizers distalized nephrons. This demonstrates that human iPSC derived nephrons respond to canonical WNTs by upregulating a transcriptional response akin to that observed in vivo. However, it is surprising that no evidence was found for an endogenous canonical WNT ligand-receptor pairing in control organoids during the timeframe when nephrons form and pattern. As part of the standard differentiation protocol cells are pulsed with GSK3β-inhibitor CHIR at day 7 and nephrons emerge 3 days later at day 10. There is therefore a discrepancy between when β-catenin signaling is driven and when nephrons form and the timing disagrees with the proposed in vivo developmental timeline for the PTA-RV progression. In vitro during days 10-14, expression of WNT4 and FGF8 (thought to be direct β-catenin targets) was low by bulk RNA sequencing (samples sequenced to ~20M reads) and by single cell RNA sequencing (~2500-4000 genes per cell). It is therefore considered neither to be actively expressed. How then do nephrons form? In mice, ectopic Notch signaling can compensate in Wnt-deficient backgrounds when a dominant active Notch intracellular domain is overexpressed. In vivo experiments in mice have also shown that the nephrogenesis and activation of Wnt4 and Lhx1 require expression of FGF8. In metanephric mesenchyme knockouts of FGF8, NPCs aggregate into PTAs and RVs but lack Wnt4 and Lhx1 expression indicating FGF8 is required during canonical Wnt activation or prefigures NPCs to be response. In recent work removing FGF8 in the NPC population, the NPCs are loosely packed but consistent with the full metanephric mesenchyme knockouts, removing FGF8 using either Pax8 or Wnt4^{Cre} lines shows PTAs still form. This agrees with the in vivo human results where FGF8 is present in the NPC population and is enriched in the CD-adjacent cells and FGF8 expression precedes broad activation of WNT4 in the PTA, in CD-adjacent cells at the WNT11/WNT9B transition point. While this is therefore consistent with FGF8 contributing to cell aggregation it also demonstrates that it is unlikely to be an absolute requirement.

Of note, while canonical WNT ligands are not detected in organoids, increased expression of WNT pathway modifiers SFRP1, SFRP2, DKK1, DKK3 are detected between days 10 and 12. These are genes often associated with β-catenin signaling and it remains possible that a basal level of canonical WNT signaling is occurring that cannot be detected with current methods. However, given that the organoids displayed expression of WNT receptors and co-receptors favor the hypothesis that they are prefigured to receive a canonical WNT, do not normally do this due to the lack of ligand inputs, and are highly when ectopic WNTs are provided. These responses are possible to detect using the methods used in this study.

In addition to the transcription response, it was also shown that organoid nephrons align their epithelial axes to towards the WNT source. Activation of β-catenin in NPC culture settings in vitro have shown that this is sufficient to drive a cell aggregation program similar to that observed during NPC aggregation into PTAs. NPCs with activated β-catenin signaling become more motile and adhesive to other differentiating NPCs. In vivo, the NPCs move over distance to leave the NPC niche and join each forming the nephron. As outlined above, the dynamics of FGF8 expression together with phenotypes makes this an unlikely candidate for being the NPC chemoattractant. The data raises the possibility that a canonical WNT acts as one of the attractive signals that coax NPCs to leave their niche and aggregate, but it does not explain how NPCs are recruited from the NPC niche into the proximal ends of forming RVs (where there is no or little WNT9B). NPC behavior is highly dynamic and they are capable of extending long cellular processes from their positions within the NPC niche to forming nephrons; indicating they respond to a long-range chemotactic signal. The alignment of organoid nephrons to the WNT source suggests that WNT9B can act as a chemoattractive signal and simultaneously regulates a transcriptional distalizing program. From the perspective of engineering a cell-derived artificial kidney, the duality of one signal driving two processes would be advantageous as it can be used to drive distal nephrons towards defined positions in a common epithelial drainage system.

Disclosed herein is that nephrons possess a previously undefined axial polarity, show its relationship to a cell organizer, and demonstrate that stem cell derived nephrons can replicate in vivo differentiation and morphogenetic programs in response to synthetic cell organizer. This example provides a framework for understanding human nephron development and provides strategies for engineering an artificial kidney that drains urine to a common epithelium.

### Materials and Methods

Materials: Antibodies: EMX2 Polyclonal Sheep IgG, (Biotechne, Cat #AF6470), DLL1 monoclonal (251127) mouse IgG2B (R&D Systems, Cat #MAB1818), JAG1Polyclonal Goat IgG (Novus, Cat #AF599), LEF1 monoclonal (C18A7) Rabbit IgG, (Cell Signaling Technologies, Cat #2286S), HNF1B monoclonal (CL0374) mouse IgG1, (Thermo Scientific, Cat #MA5-24605), POU3F3 polyclonal goat IgG, (Novus, Cat #NBP1-49872), POU3F3 polyclonal rabbit IgG, (Thermo Scientific, Cat #PA5-64311), CDH1 monoclonal (36) mouse IgG2a, κ, (BD Laboratories, Cat #610181), ZO-1 monoclonal (ZO1-1A12) mouse IgG1, (Thermo Scientific, Cat #33-9100), TFAP2A monoclonal (3B5) mouse IgG2b κ, (Santa Cruz Biotechnology, Cat #sc-12726), GFP polyclonal chicken IgY, (abcam, Cat #ab13970), WT1 monoclonal (CAN-R9(IHC)-56-2) rabbit IgG, (abcam, Cat #ab89901), Donkey-anti-Rabbit IgG AlexaFluor488, (Thermo Scientific, Cat #A-21206), Donkey-anti-Goat IgG AlexaFluor555, (Thermo Scientific, Cat #A-21432), Donkey-anti-Rabbit IgG AlexaFluor647, (Thermo Scientific, Cat #A-31573), donkey-anti-Chicken IgY (IgG) AlexaFluor488, (Jackson, Cat #703-545-155), Donkey-anti-Sheep IgG AlexaFluor488, (Jackson, Cat #713-545-147), Donkey-anti-Rabbit IgG AlexaFluor555, (Abcam, Cat #ab150074), Rat-anti-Mouse IgG2b AlexaFluor647, (Abcam, Cat #ab172327), Rat-anti-Mouse IgG2a AlexaFluor647, (BioLegend, Cat #407116), Rat-anti-Mouse IgG1 AlexaFluor647, (BioLegend, Cat #406618), Bacterial and virus strains: Escherichia coli HST08 strain (Stellar Competent Bacteria), (Takara Bio, Cat #636763), Human Kidney samples, Chemicals, peptides, and recombinant proteins: Doxycycline hydrochloride, (Sigma Aldrich, Cat #D3447-500MG), Puromycin dihydrochloride, (Thermo Scientific, Cat #A1113803), Geneticin (G418 sulfate), (Thermo Scientific, Cat #11811023), Y-27632, (STEMCELL Technologies, Cat #72304), CHIR99021, (Tocris, Cat #4423), FGF9, (Biotechne, Cat #273-F9-025), Heparin, (Sigma Aldrich, Cat #H4784), Benzyl alcohol, (Sigma Aldrich, Cat #402834-500ML), Benzyl benzoate, (Sigma Aldrich, Cat #B6630-1L), Experimental models: Cell lines: iPSC LINE IDs, e.g. HNF4A::YFP, T-REx-293, (ThermoFisher Scientific, Cat #R71007), P-cells/HEKCdh3, HEKCdh3-Wnt3a, HEKmCherry-Cdh3 (R1C3mC), HEKmCherry-Cdh3-diWnt3a (R1C3mC;R26W3a), HEKmCherry-Cdh3-diWNT9B (R1C3mC;R26W9B), Oligonucleotides: pUC19_BB_F, pUC19_BB_R, Rogi1_5HA_F1, Rogi1_5HA_R, Rogi1_3HA_F, Rogi1_3HA_R2, MCS_top, MCS_bot, GA_mes-EF1a_F, GA_hEF1a_R, GA_ef1a-mCherrySURF_F, GA_mCherrySURF2A_R, GA_2aCDH3_F, GA_mcs-CdhpA_R, Rogi1gRNA_top, Rogi1gRNA_bot, GA_TagBFP_F, GA_TagBFP_R, GA_2A_R, GA_mWnt3a_F, GA_mWnt3a_R, hROSA26gRNA_top, hROSA26gRNA_bot, LKO.1 5′, Recombinant DNA: pSpCas9-GFP, (Addgene, Cat #48138), pSpCas9-ROGI1Gma, pSpCas9-ROSA26gRNA, pHDR-ROGI1-MCS, pHDR-ROGI-mCherry^{Surface}-2A-Cdh3, pHDR-ROSA26-MCS, pHDR-ROSA26-TagBFP-2A-Wnt3a, pHDR-ROSA26-TagBFP-2A-WNT9B201, Software and algorithms: GraphPad Prism, ImageJ/FiJi, Imaris (version 10.1.1), Oxford Instruments, RStudio Posit, FW20-FastWells 20 mm Diameter×1.0 mm Depth Grace, (Bio-Labs, Cat #664113), Micro Cover Glasses, Round, No. 1, Diameter=25 mm, (VWR, Cat #48380-080), Scintillation Vials, Borosilicate Glass, (VWR, Cat #66022-081).

Cell culture: Human iPSCs were grown on matrigel-coated (Corning: 354277) 6-well plates in E8 medium (Gibco: A1517001). Kidney organoids were derived using a previously published differentiation protocol. HEK-293 cells were cultured in Dulbecco's Modified Eagle Medium (DMEM; Gibco: 11965092) supplemented with 10% fetal bovine serum (FBS; Genesee Scientific: 25-514, lot P085877), 2 mM L-glutamine (Gibco: A2916801), 100U/ml; 100 μg/ml penicillin/streptomycin (Gibco: 15140122) and 0.1 mM β-mercaptoethanol (Sigma Aldrich: M3148-25ML). HEK-293 cultures were grown to 90% confluence and passaged every 3-4 days at a 1:10-1:12 ratio. HEK-293 cells were harvested by aspirating culture medium, washing with phosphate-buffered saline (PBS; Gibco: 10010049), incubating with 0.35% trypsin (Genesee Scientific: 25-510) at 37° C. for 3-5 minutes, neutralizing with double the volume of fresh medium, and collecting the desired ratio for passaging and organizer production.

DNA constructs: For ROSA26/ROGI1 locus targeting, gRNA-encoding oligos were cloned into pSpCas9-GFP as described previously. Plasmids were screened via Sanger sequencing using the LKO.1 5′ primer. For insertion of TagBFP-2A-Wnt3a/WNT9B into ROSA26, TagBFP was PCR-generated from an in-house construct (pTH11); 2A was added downstream through two PCR rounds using sequential reverse primers. Wnt3a cDNA was PCR-generated from +cWnt3a-SO gDNA. WNT9B isoform-201 cDNA was acquired as a synthetic gene fragment following human codon GC-content optimization (Twist Biosciences). TagBFP/WNT fragments were assembled into KpnI-linearized pHDR-ROSA26-MCS vector established previously (FG's thesis, gift from Jamie Davies). For insertion of mCherry^{surface}-2A-Cdh1/Cdh3 into ROGI1, the EF1α promoter was PCR-generated from pHDR-ROSA26-MCS, mCherry^{Surface}-2A from an in-house construct (pMMP250-Cdx2), and Cdh1/Cdh3-pA from E-cell/P-cell gDNA. EF1α/mCherry^{Surface}/Cdh fragments were assembled into BamHI-linearized pHDR-ROGI1-MCS vector established in-house based on published reports. All PCRs were performed using Q5 polymerase master mix (NEB: M0492S); primer sequences are in the key resources table. Assemblies were performed using 30 fmol vector, 50 fmol per insert, HiFi DNA assembly reaction mix (NEB: E2621L), in 20 μl final volumes at 50° C. for an hour. Volumes corresponding to 2 fmol of vector were used to transform Stellar competent bacteria via 42° C. heat-shock for 30-35 seconds; 9 parts SOC medium were added to 1 part bacteria, transformants were outgrown at 37° C., 250 rpm for 40 minutes, and one-tenth of the outgrowth was plated on Luria-Bertani agar plates containing 100 μg/ml ampicillin. Four-to-six colonies were expanded in 3-4 ml ampicillin-supplemented broth and purified plasmids were screened via restriction digestions and full plasmid sequencing (Primordium labs).

Cell engineering: One-microgram pSpCas9-gRNA and 3 g pHDR-ROSA26/ROGI1 constructs were added into 125 μl OptiMEM together with 5 μl P3000 reagent. The mixture was added into a pre-vortexed solution containing 125 μl OptiMEM and 5 μl Lipofectamine3000 reagent (ThermoFisher: L3000001). Solutions were incubated on site for 15 minutes. HEK-293 6-well cultures at 70-80% confluence were gently washed in PBS, replenished with 1 ml OptiMEM, and transfection solutions were decanted drop-wise while rocking the plate. Transfectants were incubated at 37° C., 5% CO₂ for 6 hours, and replenished with culture medium. Two days later, cultures were refed with medium containing 10 μg/ml puromycin (pHDR-ROSA26-WNT) or 800 μg/ml G418 (pHDR-ROGI1-Cdh). Drug-supplemented medium was replenished every 2-3 days for 12-14 days total, whereupon 1 μM doxycycline was added for 24 hours. Cells were sorted on the basis of mCherry+TagBFP+ profile, cultured for 5 days, and re-sorted on the basis of mCherry+TagBFP− profile.

Evaluation of organizer doxycycline tuneability: One-hundred-thousand cells were seeded in 12-wells. The next day, cultures were refed with fresh medium supplemented with 10-fold serial dilutions of doxycycline. Twenty-two hours after supplementation, cultures were harvested, resuspended in 0.6 ml PBS-2% FBS, filtered through flow cytometry tube cap filters and analyzed on a BD FACSAria II cytometer.

Synthetic organizer production: On organoid differentiation day 11, HEK cultures were harvested and cell concentrations determined using a Countess II FL automated cell counter (Invitrogen). Five-thousand cells per spheroid times desired number of spheroids were pelleted and resuspended in doxycycline-supplemented medium (50 μl per 5×10³ cells). Fifty microliters were decanted in Nunclon Sphera 96U-well round bottom plate wells (Thermo Scientific 174929), the plate was centrifuged at 100 g for 2 minutes, and placed in a 37° C., 5% CO₂ incubator overnight.

Single-cell RNA sequencing and analyses: Microdissected organizer-coupled organoid regions were dissociated with Accumax (STEMCELL Technologies 07921) for 20-30 minutes, neutralized with double the amount of automacs buffer, filtered through flow cytometry cap filters and sorted for mCherry-, DAPI-DRAQ5+ live cells. Cells were processed for the split-seq Evercode WT v2 single-cell RNA-sequencing platform (Parse Biosciences, ECW02030). Samples were prepared for paired-end 150nt sequencing using NovaSeq X Plus. Fastq files were demultiplexed, quality controlled, and aligned (hg38 Ensembl 105 annotation), using the splitpipe workflow (Parse Biosciences). High-quality cells (>500 genes, <35% mitochondrial gene content) were kept using the Seurat 5.0 package. Samples were integrated using the Seurat integration function. Differentially expressed genes (>25% cells, >0.25-fold change) were identified using the FindAllMarkers function. Clusters were identified based on published nephron and interstitial markers. Day 10, 12, and 14 kidney single cell RNA sequencing was performed. Developing kidney single cell profiles (week 14 and 17 post conception) were obtained from published datasets and integrated to generate the in vivo nephrogenesis framework.

Spatial transcriptomic analyses of developing human kidneys: Whole kidneys were fixed at 4° C. for 4 hours in 4% PFA (Electron Microscopy Sciences, 15710), thoroughly washed with PBS, and embedded in OCT. 10 um sections were prepared for Xenium and GenePS platforms as per manufacturers' recommendations (10× Genomics, Spatial Genomics). Gene probesets (249-250 genes) were designed to capture all kidney populations at week 16 and the nephrogenic gene expression program. Nephrogenic niches and stages were identified based on morphologies and gene expression patterns. Transcript positions were quantified using R-studio and Fiji to examine transcript coordinates relative to the collecting duct tip and stalk using WNT11 and WNT9B as markers.

RNA scope: Whole kidneys fixed and prepared as for spatial transcriptional analyses were processed for RNA scope as previously described.

Quantification of WNT4 RNAscope polarity: RNAscope images of 8 renal vesicle-staged nephrons from one week 17.5 kidney stained for WNT4, WNT11, WNT9B, KRT8 and DAPI were processed on ImageJ/FiJi. The nephron's lumen center and WNT11/WNT9B boundary were marked with the point tool. WNT11, WNT9B and WNT4 puncta were registered using the multi-point tool. Lines were drawn between the nephron's lumen center point and the WNT11/WNT9B boundary using the straight-line tool, and between the nephron's lumen center point and WNT puncta using a custom macro. The angle of each line relative to the lumen center was recorded, summarized and averaged for all nephrons, binned and graphed in a radar plot using a custom Python script.

Quantification of spatial transcriptional data in the NPC population and PTA along an adjacent-distant axis: Spatial transcriptomics data from capturing a total of 9 CD tips and nephron progenitors with associated PTAs, from week 13 and week 16 kidneys, were analyzed for expression of PCDH15, MEOX1, ELAVL4, and FGF8. The distribution of transcripts was graphed in relation to the surface of each WNT11⁺ CD tip. Pretubular aggregates were defined based on morphologies (DAPI) and the collective view offered by expression of PCDH15, MEOX1, ELAVL4, FGF8, WNT4, LHX1, JAG1, EMX2, GATA3, POU3F3, HNF1B, LEF1, PDGFRB, in relation to the WNT11/WNT9B transition in the CD. Transcript distribution was plotted in PTAs for WNT4, FGF8, LHX1, EMX2, JAG1, MEOX1, and PCDH15 with respect to the forming lumen in the PTA and the WNT11/WNT9B transition point. The angle of each line relative to the lumen center was recorded, summarized and averaged for all nephrons, binned and graphed in a radar plot using a custom Python script.

Quantification of morphoattraction: Organizer-coupled HNF4A::YFP organoids were imaged via widefield fluorescence microscopy on Day18 and processed on ImageJ/FiJi. The center of the mCherry+ organizer was marked with the point tool, arrows were drawn from YFP+ tubules to YFP− tubules in clearly polarized nephrons showing elongated morphologies around the organizer, and axial paths were extrapolated using the straight-line tool. If paths passed through the organizer, they stopped at the organizer's end; if not, they stopped when they could not converge with any other extrapolated path. Intersections were registered using the multi-point tool. XY coordinates of the organizer's center and intersections were extracted and the Euclidean distance between each intersection and the organizer's center calculated and averaged per organoid.

Immunostaining & Clearing: Kidney organoids and mouse kidney explants were fixed for 20 min on ice in 4% PFA. Samples were permeabilized and blocked using PBS with 1.5% SEA Block (Thermo Fisher Scientific, 37527X3) and 0.1% TritonX100 (EMD Millipore, 1.08643) at 4° C. with gentle movement for 1 hour. Samples were stained with primary antibodies in blocking solution overnight. Stained samples were washed for at least 3 hours through several rounds of PBS-0.1% TritonX100 washes, and stained with secondary antibodies in blocking solution overnight. PBS washing steps were repeated the next day and nuclei were counterstained using PBS-0.1% TritonX100 containing 1 μg/mL Hoechst 33342 (Thermo Fisher Scientific, H3570) for 25 minutes. For widefield microscopy, samples were washed for 1 hour in PBS and mounted on slides with a glass coverslip in Immu-Mount (Thermo Fisher Scientific, 9990402). For confocal microscopy, organoids were cleared by gradual dehydration in and placed in BABB.

Confocal Imaging & Volume Analysis: Optically-cleared organoids were mounted in 20 mm-diameter FastWell frames (Grace Bio-Labs, #664113) loaded with 190 μl benzyl alcohol-benzyl benzoate (BABB). A coverslip shard was put on top of the transwell to ground the organoid, another round coverslip was used to seal the FastWell, and the sealed chamber was nail polish-glued to a Superfrost plus microscope slide. Organoids were imaged on a Leica SP8× confocal laser scanning microscope by stitching 20 Z-stack tiles captured using 40× oil lens (512×512 resolution). For thicker organoids a 25× water long-working distance lens was used. Imaging files extracted as TIFs were converted to IMS format using Imaris file converter and processed on Imaris imaging analysis software. Organizer surfaces were rendered from manually drawn regions on the basis of strong CDH3 or faint TFAP2A signal; a positive mask was extracted from the surface (organizer channel), and negative masks subtracting the organizer from all other channels were produced. Surfaces from resulting organizer-subtracted channels were rendered with a minimum 2000 μm³ volume threshold, and manually curated by deleting objects stemming from the mesenchyme, organizer leftover signal, and the autofluorescent transwell. Statistics and surface galleries were extracted via the Vantage tab. 3D renders were visualized using the Blend option and images captured via the Snapshot tab.

Statistics: The exact sample size (n) and statistic test employed for each dataset are detailed in the key numbers table. Repeated measurements were performed when different nephrons from the same organoid were quantified, when different immunostain intensities from the same nephron region were extracted, and when different metrics from the same image were extracted. Unless otherwise noted, data were assumed to be normally distributed. Significance and effect sizes are noted on figure panels (P: P-value, fc.: fold-change, D: Cohen's d calculated), Means and standard deviations are described in text. One-tailed t-tests were performed if the variable tested was predicted to exert a directional change.

### Example 3: Extending Inducible WNT Expression to Pluripotent Stem Cells and Ureteric Organoids

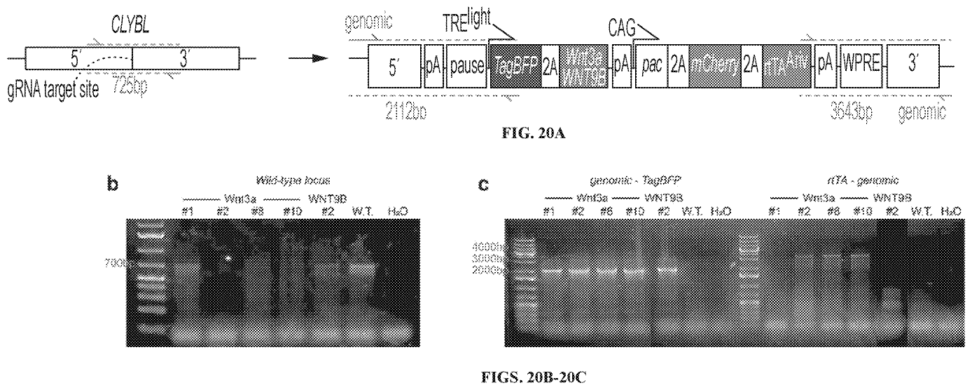

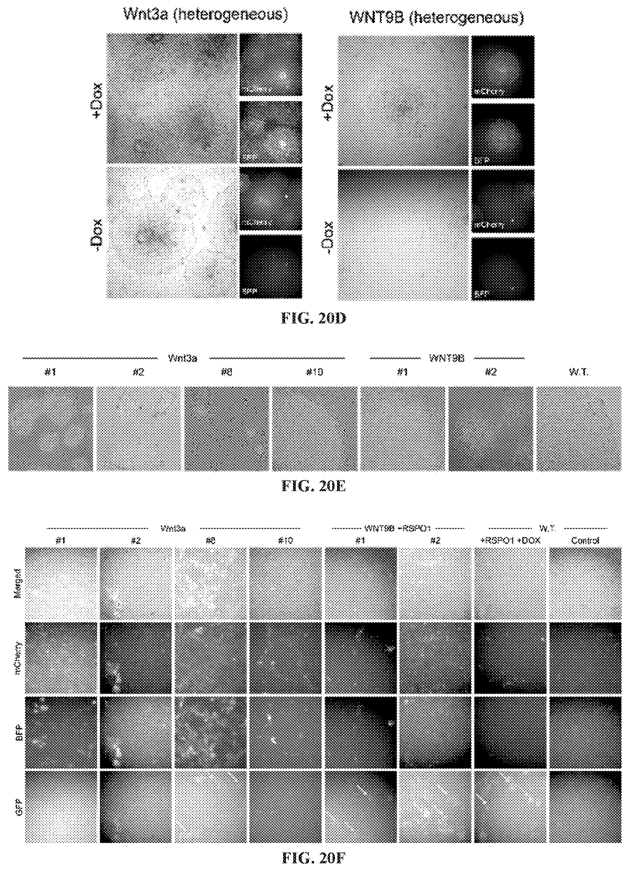

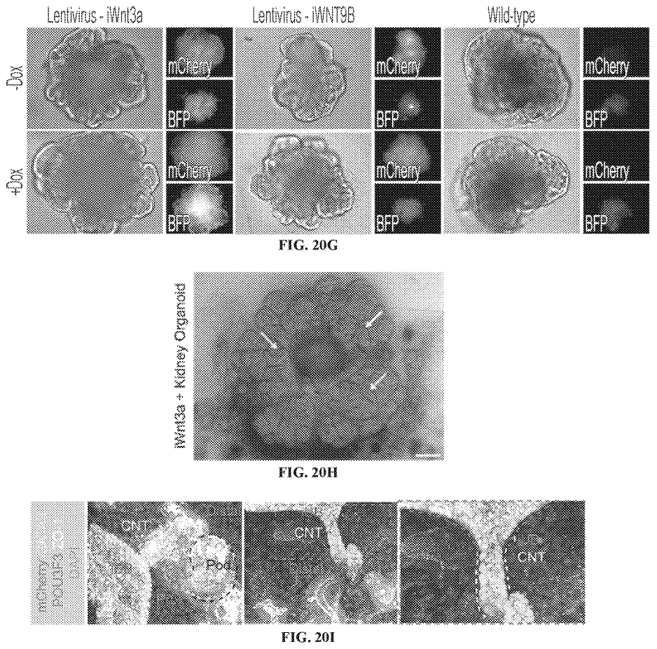

To extend WNT organizing activity to pluripotent stem cells, the study employed CRISPR to knock-in doxycycline-inducible Wnt3a/WNT9B circuits into the safe harbor CLYBL locus of H1 embryonic stem cells. A constitutive CAG promoter maintains expression of a puromycin selection marker, an mCherry fluorescent marker, and a doxycycline-regulated transactivator of the WNT transgene reported by TagBFP (FIG. 20A). Genotyping based on genomic DNA for the intact locus revealed successful disruption of CLYBL, based on a 725 bp band abundant in wild-type cells but not in a select panel of modified clones (FIG. 20B); a fainter 725 bp band in some clones suggests disruption of one allele or a heterogeneous population. Reciprocally, genotyping using primers aligning to genomic CLYBL sites and the constitutive transactivator or inducible WNT cassettes revealed several clones with site-specific integration of the circuit, based on 2.1 kb (WNT cassette) and 3.6 kb (transactivator cassette) bands (FIG. 20C). To test functionality, heterogeneous populations of H1 cells edited with Wnt3a or WNT9B circuits were cultured in the presence or absence of doxycycline and monitored for fluorescence. Live epifluorescence microscopy showed mCherry+ colonies in either condition, and a marked increase in BFP fluorescence following 40 hours of doxycycline exposure (FIG. 20D). Using this strategy, the study established a select panel of clones with stable mCherry fluorescence over prolonged passaging (FIG. 20E).

To test for functional secretion of WNT ligands, the study co-cultured clones with TCF/LEF::eGFP reporter mouse embryonic stem cells in the presence of doxycycline on the premise that functional WNT ligands from producer H1 cells will induce GFP in receiver mouse cells via the Wnt/β-catenin/TCF-LEF signaling pathway. While different H1 clones showed variation in fluorescence expression following a 2-day co-culture under conditions not supporting human pluripotency, Wnt3a clone 8 showed notable BFP fluorescence coupled to presence of GFP in adjacent mouse reporter cells (FIG. 20F). GFP was also seen in co-cultures with WNT9B clones in the presence of RSPO1, a potentiator of the Wnt/β-catenin pathway, although this was also seen in co-cultures with wild-type H1 cells (FIG. 20F). Overall, these results present a strategy for the integration of doxycycline-inducible WNT circuits into the CLYBL locus of human pluripotent stem cells, which permits stable transgene expression and functional WNT signaling to receiver cells upon doxycycline induction, in a subset of engineered clones.

To extend WNT organizing activity to ureteric organoids (iUBs), the study differentiated wild-type H1 cells into iUBs, and engineered iUBs with lentiviruses harboring a variant of the iWNT circuit (PGK promoter instead of CAG for transactivator cassette; entire circuit within LTR-flanked lentivirus cargo construct). iWnt3a iUBs showed mCherry fluorescence irrespectively of doxycycline supplementation, and a marked increase in BFP fluorescence when exposed to doxycycline; iWNT9B and wild-type iUBs did not show notable BFP (FIG. 20G). When co-cultured with kidney organoids, iWnt3a iUBs elicited nephron formation in a local radius around the iUB, with lumenized tubular morphologies emerging nearby and orienting themselves towards iWnt3a iUB (FIG. 20H, yellow arrows). Immunostains revealed mCherry+ iWnt3a-iUBs fusing with nephron GATA3+ connecting tubules that lie in continuum to POU3F3+ distal tububles extending from globular ZO-1-demarcated renal corpuscle morphologies (FIG. 20I). These experiments present a viable strategy to engineer inducible WNT circuits in pluripotent stem cells and derivative ureteric organoids, that can successfully signal to surrounding tissues, for purposes of inducing WNT-mediated nephron polarization, morphoalignment, and tubular anastomosis.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

1. A synthetic organizer comprising one or more cells, wherein the one or more cells comprise a nucleic acid construct comprising a WNT3A gene or a WNT9B gene under the control of an inducible promoter.

2. The synthetic organizer of claim 1, wherein the one or more cells comprise kidney cells.

3. The synthetic organizer of claim 2, wherein the kidney cells comprise human embryonic kidney (HEK) cells, a collecting duct cell, or an iPS-derived ureteric bud precursor cell.

4. The synthetic organizer of claim 1, wherein the one or more cells comprise pluripotent stem cells.

5. The synthetic organizer of claim 4, wherein the pluripotent stem cells comprise embryonic stem cells, mesenchymal stem cells, or induced pluripotent stem cells.

6. The synthetic organizer of claim 1, wherein the one or more cells comprise a ureteric organoid (iUB).

7. The synthetic organizer of claim 1, wherein the nucleic acid construct is transfected into the one or more cells.

8. The synthetic organizer of claim 1, wherein the nucleic acid construct is inserted into the genome of the one or more cells.

9. The synthetic organizer of claim 1, wherein the inducible promoter is a Tet promoter.

10. The synthetic organizer of claim 1, wherein the one or more cells comprise an additional nucleic acid construct comprising a CDH3 or CDH1 gene.

11. The synthetic organizer of claim 10, wherein the additional nucleic acid construct is transfected into the one or more cells, or inserted into the genome of the one or more cells.

12. A system comprising a synthetic organizer and a human kidney organoid nephron, wherein the synthetic organizer facilitates patterning of the human kidney organoid nephron.

13. The system of claim 12, wherein the synthetic organizer comprises one or more cells comprising kidney cells, pluripotent stem cells, or ureteric organoids (iUBs).

14. The system of claim 13, wherein the one or more cells comprise a nucleic acid construct comprising a WNT3A gene or a WNT9B gene under the control of an inducible promoter.

15. The system of claim 14, wherein the one or more cells comprise an additional nucleic acid construct comprising a CDH3 or CDH1 gene.

16. A method of patterning a human kidney organoid nephron, comprising:
obtaining a human kidney organoid nephron; and
contacting the human kidney organoid nephron with a synthetic organizer.

17. The method of claim 16, wherein the synthetic organizer comprises one or more cells comprising kidney cells, pluripotent stem cells, or ureteric organoids (iUBs).

18. The method of claim 17, wherein the one or more cells comprise a nucleic acid construct comprising a WNT3A gene or a WNT9B gene under the control of an inducible promoter.

19. The method of claim 18, wherein the one or more cells comprise an additional nucleic acid construct comprising a CDH3 or CDH1 gene.

20. The method of claim 16, further comprising a step of detecting expression of a distal lineage gene marker, wherein the expression of the distal lineage gene marker signals the presence of distal nephron cells.
